(19) United States
(12) Patent Application Publication
(10) Pub. No.: US 2013/0217721 A1
(43) Pub. Date: Aug. 22, 2013

(54) COMPOSITIONS AND METHODS FOR DETECTION AND TREATMENT OF B-RAF INHIBITOR-RESISTANT MELANOMAS

(71) Applicants: Roger S. Lo, Los Angeles, CA (US); Antoni Ribas, Los Angeles, CA (US)

(72) Inventors: Roger S. Lo, Los Angeles, CA (US); Antoni Ribas, Los Angeles, CA (US)

(73) Assignee: The Regents of the University of California, Oakland, CA (US)

(21) Appl. No.: 13/879,302

(22) PCT Filed: Nov. 18, 2011
(86) PCT No.: PCT/US2011/061552

§ 371 Date: Apr. 12, 2013

Related U.S. Application Data
(60) Provisional application No. 61/415,417, filed on Nov. 19, 2010.
(60) Provisional application No. 61/547,026, filed on Oct. 13, 2011.

(51) Int. Cl. G01N 33/574; A61K 31/437; A61K 45/06; C12Q 1/68

(52) U.S. Cl. CPC G01N 33/5743; C12Q 1/6869; C12Q 1/6874; A61K 31/437; A61K 45/06
USPC 514/300; 435/6.12; 435/7.9; 506/9

(57) ABSTRACT

Specific, targetable molecules mediating acquired resistance of B-RAF-mutant melanomas to a B-RAF inhibitor, thereby providing materials and methods for the treatment and detection of B-RAF inhibitor resistant cancers, such as melanoma. A method of identifying a patient to be treated with an alternative to B-RAF inhibitor therapy is described. Also described is a method of treating a patient having cancer. The patient is administered a MEK inhibitor, optionally in conjunction with vemurafenib therapy, or an inhibitor of the MAPK pathway (RAF, MEK, ERK) in conjunction with an inhibitor of the RTK-PI3K-AKT-mTOR pathway.

This application claims the benefit of U.S. provisional patent application No. 61/415,417, filed Nov. 19, 2010, and U.S. provisional patent application No. 61/547,026, filed Oct. 13, 2010, the entire contents of each of which are incorporated herein by reference. Throughout this application various publications are referenced. The disclosures of these publications in their entireties are hereby incorporated by reference into this application in order to describe more fully the state of the art to which this invention pertains.

### STATEMENT REGARDING FEDERALLY SPONSORED RESEARCH

This invention was made with Government support of Grant No. CA151638, awarded by the National Institutes of Health. The Government has certain rights in this invention.

### TECHNICAL FIELD

The present invention relates generally to detection, diagnosis, monitoring and treatment of cancer, such as melanoma. The invention more specifically pertains to B-RAF inhibitor-resistant cancers and selection of effective treatment strategies.

### BACKGROUND

Activating B-RAF V600E kinase mutations occur in ˜7% of human malignancies and ˜60% of melanomas. Early clinical experience with a novel class I RAF-selective inhibitor, PLX4032, demonstrated an unprecedented 80% anti-tumor response rate among patients with ^{V600E}B-RAF-positive melanomas, but acquired drug resistance frequently develops after initial responses. There is thus a need to discover mechanisms of melanoma escape from B-RAF inhibition that can be demonstrated in tumors from human subjects.

There remains a need for improved tools to permit the detection, identification and prognosis of drug resistant cancers, particularly B-RAF inhibitor-resistant melanomas. There also remains a need for targets useful in the detection and treatment of cancer.

### SUMMARY

The invention meets these needs and others by describing specific, targetable molecules mediating acquired resistance of B-RAF-mutant melanomas to a specific B-RAF inhibitor (PLX4032) in both in vitro models and patient-derived tissues, thereby providing materials and methods for the treatment and detection of B-RAF inhibitor resistant cancers. In one embodiment, the invention provides a method of identifying a patient to be treated with an alternative to B-RAF inhibitor therapy. The method comprises (a) assaying a sample obtained from the patient for a measure of B-RAF inhibitor resistance, (b) selecting samples that exhibit B-RAF inhibitor resistance; and (c) identifying a patient whose sample was selected in (b) as a candidate for alternative therapy. In a typical embodiment, the measure of B-RAF inhibitor resistance is selected from: (1) an alternative splice variant or gene amplification of ^{V600E}B-RAF; (2) elevated levels of PDGFR-beta; (3) an activating mutation of N-RAS; and (4) an activating mutation of AKT1.

In one embodiment, the assaying for an alternative splice variant of ^{V600 E}B-RAF comprises amplification of ^{V600E}B RAF. Amplification of ^{V600E}B-RAF and detection of alternative splice variants of ^{V600E}B-RAF can be performed using standard techniques known to those skilled in the art. Detection of one or more alternative splice variants comprises, for example, analysis of protein expression, whereby presence of a variant of the 90 kD ^{V600E}B-RAF is indicative of B-RAF inhibitor resistance. In one specific example, the presence of a B-RAF variant of approximately 61 kD is indicative of B-RAF inhibitor resistance. In another example, detection of one or more alternative splice variants comprises polymerase chain reaction (PCR) analysis of cDNA, DNA or RNA isolated from the sample obtained from the patient, whereby presence of a transcript that differs from the single 2.3 kb transcript representing full-length B-RAF is indicative of B-RAF inhibitor resistance. In one specific example, the presence of a transcript of approximately 1.7 kb is indicative of B-RAF inhibitor resistance. In some embodiments, the PCR is quantitative PCR or Q-PCR.

In one embodiment, the assaying for PDGFR-beta comprises assaying for PDGFR-beta mRNA, protein or phospho-protein. Assays for mRNA, protein and phospho-protein can be performed using techniques well-known to those skilled in the art. For example, conventional northern blots, western blots, dot blots, and immunoblots can be used. Detection of increased levels of PDGFR-beta relative to a control is indicative of B-RAF inhibitor resistance. In one embodiment, the assaying for hyperactivity of PDGFR-beta comprises measuring phospho-tyrosine levels on PDGFR-beta hyperactivity. An increased level of phosphor-tyrosine relative to a control is indicative of B-RAF inhibitor resistance. In one embodiment, an elevated or increased level is at least 50% more than control. In another embodiment, an elevated or increased level is at least 2-fold more than control. In some embodiments, elevated or increased is at least 5-fold or 10-fold more than control.

In one embodiment, the assaying for an indicator of N-RAS mutation comprises assaying for an activating N-RAS mutation. Example of activating N-RAS mutations include missense mutations at codon 12, 13 and 61, such as Q61K or Q61 R. In some embodiments, the assaying for an indicator of N-RAS mutation comprises assaying for elevated levels of N-RAS gDNA, mRNA or protein copy number.

In one embodiment, the assaying for an indicator of AKT1 mutation comprises assaying for an activating AKT1 mutation. Examples of activating AKT1 mutations include missense mutations that result in a Q79K amino acid substitution. In one embodiment, the assaying for an activating mutation of AKT1 comprises measuring phospho-AKT1 levels.

The method can be performed prior to B-RAF inhibitor therapy, and/or after initiation of B-RAF inhibitor therapy. In one embodiment, the B-RAF inhibitor is vemurafenib. The sample obtained from the patient can be a biopsy or other clinical specimen obtained, for example, by needle aspiration or other means of extracting a specimen from the patient that contains tumor cells. The sample can also be obtained from peripheral blood, for example, by enriching a sample for circulating tumor cells.

Examples of alternative therapy include, but are not limited to, augmenting B-RAF inhibitor therapy with at least one additional drug. The additional drug can include a MAPK/ERK kinase (MEK) inhibitor, such as PD0325901,GDC0973, GSK1120212, and/or AZD6244. Another example of an additional drug is an inhibitor of the RTK-PI3K-AKT-mTOR pathway, such as BEZ235, BKM120, PX-866, and/or GSK2126458. In one embodiment, the alternative therapy comprises suspension of vemurafenib therapy.

In some embodiments, the patient has, or is suspected of having, a B-RAF-mutant cancer. In a typical embodiment, the patient has, or is suspected of having, a B-RAF-mutant melanoma.

The invention further provides a method of treating a patient having cancer, the method comprising administering to the patient a MEK inhibitor, optionally in conjunction with vemurafenib therapy, or an inhibitor of the MAPK pathway (RAF, MEK, ERK) in conjunction with an inhibitor of the RTK-PI3K-AKT-mTOR pathway. Examples of MEK inhibitors include, but are not limited to PD0325901,GDC0973, GSK1120212, and/or AZD6244. Examples of inhibitors of the RTK-PI3K-AKT-mTOR pathway include, but are not limited to BEZ235, BKM120, PX-866, and GSK2126458. In a typical embodiment, the patient has melanoma. In one embodiment, the melamona is a B-RAF-mutant melanoma. In one embodiment, the melanoma expresses a 61 kD variant of B-RAF, such as, for example, one that lacks exons 4-8.

### BRIEF DESCRIPTION OF THE FIGURES

FIGS. 1A-1B demonstrate that in vitro models of PLX4032 acquired resistance display differential MAPK reactivation. FIG. 1A, Parental and PLX4032-resistant sub-lines were treated with increasing PLX4032 concentration (0, 0.01, 0.1, 1 and 10 μM), and the effects on MAPK signalling were determined by immunoblotting for p-MEK1/2 and p-ERK1/2 levels. Total MEK1/2, ERK1/2 and tubulin levels, loading controls. FIG. 1B, Heat map for B-RAF(V600E) signature genes in each of the cell lines treated with DMSO or PLX4032. Colour scale, logₑ-transformed expression (red is 4.0 as depicted in scale across bottom, high; green is −4.0, low) for each gene (row) normalized by the mean of all samples (number after each gene indicates corresponding probeset). Blue box (right, vertical) showing M249 R4 MAPK reactivation. Yellow box (left, horizontal) showing diminished, baseline expression of B-RAF(V600E) signature genes in M229 and M238 resistant sub-lines (FDR<0.05).

FIGS. 2A-2D show that PDGFR-beta upregulation is strongly correlated with PLX4032 acquired resistance. FIG. 2A, Left, total levels of PDGFR-beta and EGFR. A431, an EGFR-amplified cell line. Tubulin levels, loading control. Right, whole-cell extracts were incubated on the RTK antibody arrays, and phosphorylation status was determined by subsequent incubation with anti-phosphotyrosine horseradish peroxidase (each RTK spotted in duplicate, positive controls in corners, gene identity below). FIG. 2B, Anti-PDGFR-beta immunohistochemistry of formalin-fixed, paraffin-embedded tissues. Prostate, negative control; placenta, positive control. Black bar, 50 μm. FIG. 2C, Relative RNA levels of PDGFR-beta in M229 P/R5 and Pt48 R as determined by real-time, quantitative PCR (average of duplicates). FIG. 2D, Total PDGFR-beta (left) and p-RTK (right) levels in Pt48 R versus M229 R5.

FIGS. 3A-3B show that N-RAS upregulation correlates with a distinct subset of PLX4032 acquired resistance. FIG. 3A, Detection of a N-RAS(Q61K) allele in M249 R4 and Pt55 R (SEQ ID NO: 159). FIG. 3B, The levels of activated RAS (aRAS) and N-RAS (aN-RAS) eluted after pull-down using the RAS-binding domain (RBD) of RAF-1. The total levels of RAS, N-RAS, PDGFR-beta and tubulin (loading control) from the whole-cell lysates are shown by immunoblotting. Effects of GDP and GTPγS pre-incubation on RBD pull-down and beads without RBD pull-down from Pt48 R lysates are shown as controls.

FIGS. 4A-4B shows that PDGFRβ- and N-RAS-mediated growth and survival pathways differentially predict MEK inhibitor sensitivity. FIG. 4A, Transduction of PDGFRβ shRNAs in M229 R5 and M238 R1 (1 μM PLX4032), RNA (relative to GAPDH) and protein knockdown, effects on p-ERK levels, cell cycle distribution, and apoptosis (when applicable). M229 R5 was also treated with 0.5 μM AZD6244. PI, propidium iodide. FIG. 4B, Transduction of N-RAS shRNAs in M249 R4 and Pt55 R (1 μM PLX4032), RNA and protein knockdown, effects on p-ERK levels and apoptosis. FIG. 4C, Survival curves for isogenic cell line pairs and melanoma cultures treated with the indicated AZD6244 concentration for 72 h (relative to DMSO-treated controls; mean±s.e.m., n=5). PLX4032-resistant cells were grown with PLX4032. Dashed line, 50% cell killing.

FIGS. 5A-5E. Resistance to the RAF inhibitor PLX4032 (vemurafenib) is associated with failure of the drug to inhibit ERK signaling. FIG. 5A. PLX4032 IC50 curves (at 5 days) for the SKMEL-239 parental cell line and five PLX4032-resistant clones. FIG. 5B. Effects of 2 μM PLX4032 on ERK signaling in parental (Par) and resistant clones (C1-5). FIG. 5C. Western blot for components of the ERK and AKT signaling pathways in parental and resistant clones (2 μM PLX4032/24 hours). FIG. 5D. Dose-response of pMEK and pERK downregulation at 1 hour to increasing concentrations of PLX4032 in parental and two representative resistant clones (C3 and C5). FIG. 5E. Graphic representation of the chemiluminescent signal intensities from 5D and determination of IC50s for inhibition of MEK phosphorylation by PLX4032 in the parental and C3 and C5 clones.

FIGS. 6A-6E. A BRAF(V600E) variant that lacks exons 4-8 is resistant to the RAF inhibitor PLX4032. FIG. 6A. PCR analysis of BRAF in cDNA from parental (P) and C3 cells. Primers were designed at the N-terminus and C-terminus of BRAF. Sequencing of the 1.7 kb product expressed in the C3 clones but not in parental cells revealed an in frame deletion of five exons (4-8) in cis with the V600E mutation. The expected protein product from the 1.7 kb mRNA has 554 amino acids and a predicted molecular weight of 61 kd. Abbreviations: CR1: Conserved Region 1, CR2: Conserved Region 2, CR3: Conserved Region 3, RBD: RAS-binding domain, CRD: Cystine-Rich Domain, AS: Activation Segment, KD: Kinase Domain. FIG. 6B. Full length wild-type BRAF and the 1.7 kb/61 kd splice variant of BRAF(V600E) were cloned into a pcDNA3.1 vector with a FLAG tag at the C-terminus and expressed in 293H cells. The effect of PLX4032 (2 μM for 1 hour) on ERK signaling in the presence of p61 BRAF(V600E) was analyzed by western blot for pMEK and pERK. FIG. 6C. To compare levels of dimerization, 293H cells co-expressing FLAG tagged and V5-tagged p61 BRAF(V600E), full length BRAF(V600E) and the corresponding dimerization-deficient mutants p61 BRAF(V600E/R509H) and BRAF(V600E/R509H) were lysed followed by immunoprecipitation with FLAG antibody. Western blots with V5 or FLAG antibodies were performed as indicated. FIG. 6D. Comparison of MEK/ERK activation and sensitivity of ERK signaling to PLX4032 (2 μM for 1 hour) in 293H cells expressing either Flag-tagged BRAF(V600E) or the dimerization mutant Flag-tagged BRAF(V600E/R509H). FIG. 6E. Constructs expressing V5-tagged BRAF(V600E), p61 BRAF(V600E) or the dimerization mutant p61 BRAF(V600E/R509H) were transfected into 293H cells and treated with DMSO or 2 μM PLX4032 for 1 hour.

FIGS. 7A-7C. Identification of splice variants of BRAF(V600E) in human tumors resistant to PLX4032 (vemurafenib). FIG. 7A. PCR analysis of cDNA derived from tumor samples using primers located at the N and C-termini of BRAF. In samples with only one band (full-length BRAF), we detected both BRAF(V600E) and wild-type BRAF (1+2). In resistant tumor samples expressing shorter transcripts, the shorter transcript was a splice variant of BRAF(V600E) (3, 4, 5). The figure shows samples from three patients with acquired resistance to PLX4032: baseline (B) and post-treatment progression (DP) samples from patients I and post-treatment samples from patients II and III. A tumor sample from a patient with de novo resistance to PLX4032 (patient IV) is also shown. The intermediate band in samples expressing splicing variants (Pt I-III) is an artifact of the PCR reaction resulting from switching between two very similar templates. Representative Sanger sequencing traces showing the junction between exons 3 and 11 in the DP sample from patient I compared to the full-length transcript derived from the baseline pre-treatment sample from the same patient (sequences are GGACAGTG/GTACCTGCA (SEQ ID NO: 160) for junction between exons 3 and 4; and GGACAGTG/AAACACTT for junction between exons 3 and 11 (SEQ ID NO: 161)). FIG. 7B. As in FIG. 7A, baseline (B) and disease progression (DP) samples from a patient with an exon 2-10 deletion (sequence for junction between exons 1 and 11 is CCGGAGGAG/AAAACACTT; SEQ ID NO: 162). RNA/cDNA levels of the exon 10 deletion were determined by real-time, quantitative PCR (normalized to GAPDH within each sample) using an exon1-exon11 junction primer. The data are shown as average of duplicates and expressed as relative levels between patient-matched samples. FIG. 7C. Exon organization of the splice variants found in tumors from six patients that relapsed on PLX4032. The variant from patient II was identical to the one identified in the C1, C3 and C4 PLX4032-resistant SKMEL-239 clones.

FIGS. 8A-8C. Exome sequencing identifies ^{V600E}B-RAF amplification as a candidate mechanism for BRAFi resistance. FIG. 8A, Copy number variations (CNVs) called from whole exome sequence data on two triads of gDNAs using ExomeCNV and chromosome 7 as visualized by Circos (outer ring, genomic coordinates (Mbp); centromere, red; inner ring, log ratio values between baseline and disease progression (DP) samples' average read depth per each capture interval; scale of axis for Pt #5-5 to 5 and for Pt #8-2.5 to 2.5). Two patients whose melanoma responded to and then progressed on vemurafenib. The genomic region coded orange represents the location of B-RAF (chr7:140,424,943-140,524,564), which shows an average log ratio value of 1.14 (2.2 fold gain; Pt #5) and 3.8 (12.8 fold gain; Pt #8). FIG. 8B, B-RAF immunohistochemistry on paired tissues derived from the same patients as in FIG. 8A. FIG. 8C, Validation of ^{V600E}B-RAF copy number gain by gDNA qPCR and recurrence across distinct patients (highlighted in lighter font). PMN, peripheral mononuclear cells, and HDF, human dermal fibroblasts for diploid gDNAs.

FIGS. 9A-9C. ^{V600E}B-RAF levels modulate melanoma sensitivity to vemurafenib. FIG. 9A, ^{V600E}B-RAF^{V600E} over-expression at various levels (left, tubulin as loading control) did not alter the pERK level in the absence of vemurafenib/PLX4032 but conferred growth resistance to the parental line, M395 P (right) when exposed to indicated concentrations of PLX4032 for 72 h (relative to DMSO-treated controls; mean±SEM, n=5). Dashed line, 50% inhibition. FIG. 9B, Transduction of shRNA to knockdown BRAF^{V600E} in the drug-resistant sub-line, M395 R, did not alter the pERK level in the absence of PLX4032 but restored growth sensitivity to PLX4032 (72 h). FIG. 9C, Increasing (in M395 P) or decreasing (in M395 R) BRAF^{V600E} levels decreased or increased pERK sensitivity to PLX4032 (0, 0.1, 1, 10 μM) treatments for 1 h, respectively.

FIGS. 10A-10F. MAPK reactivating mechanisms display differential sensitivities to targeted agents and dependency on C-RAF. FIG. 10A and FIG. 10B, Survival curves of indicated cell lines to 72 h of inhibitor treatments, showcasing differential responses at the micro-molar drug range. FIG. 10C, Indicated cell lines were treated with constant ratios of PLX4032 and AZD6244 and survival measured after 72 h. Relative synergies, expressed as log₁₀ of CI values, are shown. FIG. 10D, M249 (R4) and M395 R were seeded at single cell density and treated with indicated concentrations of PLX4032 and/or AZD6244. Inhibitors and media were replenished every two days, colonies visualized by crystal violet staining after 8 days of drug treatments, and quantified (% growth relative to cells treated with 1 μM PLX4032). Photographs representative of two independent experiments. FIG. 10E, Survival curves of indicated cell lines after shScrambled or shC-RAF transduction (inset) and when treated with PLX4032 for 72 h. FIG. 10F, Clonogenic assays of cell lines in FIG. 10E with 14 days (M249 R4) or 18 days (M395 R) of PLX4032 treatment.

FIG. 11 shows results of AKT1 gene sequencing that detected AKT1(Q79K) in tumor sample from a biopsy taken after disease progression as compared to the wild type (wt) sequence present in the tumor cells taken before B-RAF inhibitor treatment (SEQ ID NO: 1).

### DETAILED DESCRIPTION

The present invention is based on the discovery of mechanisms of acquired resistance to PLX4032/vemurafenib. This discovery enables the identification of a subset of melanoma patients treated with B-RAF-targeting agents who respond and subsequently relapse via the described mechanisms. The invention also provides for implementation of a second-line and/or combination treatment strategy via pharmacologic agents to manage a specific subset of melanoma patients relapsing on B-RAF-targeting agents, as well as patients with other types of B-RAF-related cancers who develop resistance to B-RAF-targeting agents. These mechanisms may be instructive for why other cancers with BRAF mutations may be primarily resistant to B-RAF inhibitors. These mechanisms may also arise and result in acquired (secondary) resistance in other B-RAF mutant cancers that may be primarily sensitive to B-RAF inhibitors.

The invention provides diagnostic assays tailored to detect each mechanism at the onset of clinical and radiographic evidence of acquired resistance in patients with B-RAF(V600E)-positive metastatic melanomas who are treated with B-RAF inhibitors (PLX4032/vemurafenib or other similar agents such as GSK2118436/dabrafenib) and who initially respond to B-RAF inhibitors (partial response, also referred to as RECIST). These mechanisms of acquired B-RAF inhibitor resistance are largely mutually exclusive per tumor site, but distinct foci of tumor progression may harbor distinct molecular lesions. Using clinical samples or biopsies derived from patients or short-term culture derived from such, one assay detects increased levels of PDG FR-beta transcript by quantitative RT-PCR or protein/phospho-tyrosine protein levels by immunologic assays. Another assay detects an N-RAS activating mutation (for example Q61K or Q61R, but any N-RAS activating mutation could be tested) by a gene sequencing approach. Another assay detects an approximately 61 kd splice variant of ^{V600E}B-RAF that lacks certain exons that result in deletions of variable portions of the N-terminal protein domain. Another assay detects ^{V600E}B-RAF copy number gain or amplification by methods such as FISH or quantitative PCR.

The assays can be used to stratify patients for sequential treatment strategies with B-RAF inhibitor-alternative drug(s) or combination of drugs inclusive of B-RAF inhibitors aimed at overcoming acquired B-RAF inhibitor resistance. Useful applications from this invention include, but are not limited to:
Detection of PDGFR-beta activation (or a surrogate molecular marker such as a gene signature) in a pre-existing sub-population of B-RAF-mutant melanoma tumors prior to B-RAF-targeted therapy;
Detection in patient-derived tumors or cell lines of PDGFR-beta activation as a mechanism of tumor escape from B-RAF inhibition, and corresponding therapeutic strategies targeted against PDGFR-beta-activated melanomas
Detection of N-RAS activating mutations (or a surrogate molecular signature of such activation) in a melanoma biopsy prior to B-RAF targeted therapy;
Detection of N-RAS activating mutations in melanoma tissues or cell lines that have acquired resistance to B-RAF-targeted therapy, and therapeutic strategies targeting N-RAS-activated, B-RAF inhibitor-resistant melanomas.
Detection of ^{V600E}B-RAF alternative spliced variants in a melanoma biopsy prior to B-RAF targeted therapy, and in melanoma tissues or cell lines that have acquired resistance to B-RAF-targeted therapy, as well as therapeutic strategies targeted against B-RAF inhibitor-resistant melanomas harboring ^{V600E}B-RAF alternative spliced variants.
Detection of ^{V600E}B-RAF gene amplification in a melanoma biopsy prior to B-RAF targeted therapy, and in melanoma tissues or cell lines which have acquired resistance to B-RAF-targeted therapy, as well as therapeutic strategies targeted against B-RAF inhibitor-resistant melanomas harboring ^{V600E}B-RAF gene amplification.
Similar detection and treatment strategies relating to mutation of AKT1 in melanoma tissues or cell lines that have acquired resistance to B-RAF-targeted therapy.

Definitions

All scientific and technical terms used in this application have meanings commonly used in the art unless otherwise specified. As used in this application, the following words or phrases have the meanings specified.

As used herein, “B-RAF inhibitors” refers to drugs that target an acquired mutation of B-RAF that is associated with cancer, such as ^{V600E}B-RAF. Representative examples of such a B-RAF inhibitor include PLX4032/vemurafenib or other similar agents, such as GSK2118436/dabrafenib.

As used herein, ^{V600E}B-RAF″ refers to B-RAF having valine (V) substituted for by glutamate (E) at codon 600.

As used herein, “N-RAS activating mutation” refers to any mutation of N-RAS resulting in activation of N-RAS, such as activating the potential of N-RAS to transform cells. Examples of N-RAS activating mutations include, but are not limited to, those that change amino acid residues 12, 13 or 61, such as, for example, Q61K or Q61R.

As used herein, “MAPK/ERK kinase (MEK)” refers to a mitogen-activated protein kinase also known as microtubule-associated protein kinase (MAPK) or extracellular signal-regulated kinase (ERK).

As used herein, “AKT1 activating mutation” refers to any mutation of AKT1 resulting in activation of AKT1, such as activating the potential of AKT1 to transform cells. Examples of AKT1 activating mutations include, but are not limited to, Q79K.

As used herein, “pharmaceutically acceptable carrier” or “excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well known conventional methods (see, for example, Remington's Pharmaceutical Sciences, 18th edition, A. Gennaro, ed., Mack Publishing Co., Easton, Pa., 1990).

As used herein, “a” or “an” means at least one, unless clearly indicated otherwise.

Methods for Identifying Candidates for Alternate Therapies

Methods described herein are performed using clinical samples or biopsies derived from patients or short-term culture derived from same. The methods guide the clinician in stratifying patients for sequential treatment strategies with B-RAF inhibitor-alternative drug(s) or combination of drugs inclusive of B-RAF inhibitors aimed at overcoming acquired B-RAF inhibitor resistance.

In one embodiment, the invention provides a method of identifying a patient to be treated with an alternative to B-RAF inhibitor therapy. The method comprises (a) assaying a sample obtained from the patient for a measure of B-RAF inhibitor resistance, (b) selecting samples that exhibit B-RAF inhibitor resistance; and (c) identifying a patient whose sample was selected in (b) as a candidate for alternative therapy. In a typical embodiment, the measure of B-RAF inhibitor resistance is selected from: (1) an alternative splice variant or gene amplification of ^{V600E}B-RAF; (2) elevated levels of PDGFR-beta; (3) an activating mutation of N-RAS; and (4) an activating mutation of AKT1.

One can detect an approximately 61 kd splice variant of ^{V600E}B-RAF that lacks certain exons that result in deletions of variable portions of the N-terminal protein domain. Another assay detects ^{V600E}B-RAF copy number gain or amplification by using such methods as FISH or quantitative PCR. In one embodiment, the assaying for an alternative splice variant of ^{V600E}B-RAF comprises amplification of ^{V600E}B-RAF. Amplification of ^{V600E}B-RAF and detection of alternative splice variants of ^{V600E}B-RAF can be performed using standard techniques known to those skilled in the art. Detection of one or more alternative splice variants comprises, for example, analysis of protein expression, whereby presence of a variant of the 90 kD ^{V600E}B-RAF is indicative of B-RAF inhibitor resistance.

In one specific example, the presence of a B-RAF variant of approximately 61 kD is indicative of B-RAF inhibitor resistance. In another example, detection of one or more alternative splice variants comprises polymerase chain reaction (PCR) analysis of cDNA, DNA or RNA isolated from the sample obtained from the patient, whereby presence of a transcript that differs from the single 2.3 kb transcript representing full-length B-RAF is indicative of B-RAF inhibitor resistance. In one specific example, the presence of a transcript of approximately 1.7 kb is indicative of B-RAF inhibitor resistance. In some embodiments, the PCR is quantitative PCR or Q-PCR.

In one embodiment, the assaying for PDGFR-beta comprises assaying for PDGFR-beta mRNA, protein or phospho-protein. Assays for mRNA, protein and phospho-protein (e.g., phospho-tyrosine) can be performed using techniques well-known to those skilled in the art. For example, conventional northern blots, and immunologic assays, such as western blots, dot blots, and immunoblots, can be used. One can detect increased levels of PDGFR-beta transcript by quantitative RT-PCR. Detection of increased levels of PDGFR-beta relative to a control is indicative of B-RAF inhibitor resistance. In one embodiment, the assaying for hyperactivity of PDGFR-beta comprises measuring phospho-tyrosine levels on PDGFR-betahyperactivity. An increased level of phosphor-tyrosine relative to a control is indicative of B-RAF inhibitor resistance. In one embodiment, an elevated or increased level is at least 50% more than control. In another embodiment, an elevated or increased level is at least 2-fold more than control. In some embodiments, elevated or increased is at least 5-fold or 10-fold more than control.

In one embodiment, the assaying for an indicator of N-RAS mutation comprises assaying for an activating N-RAS mutation. An N-RAS activating mutation can be detected using conventional methods, such as by gene sequencing. Examples of activating N-RAS mutations include missense mutations at codon 12, 13 and 61, such as Q61K or Q61R. In some embodiments, the assaying for an indicator of N-RAS mutation comprises assaying for elevated levels of N-RAS gDNA, mRNA or protein copy number.

In one embodiment, the assaying for an indicator of AKT1 mutation comprises assaying for an activating AKT1 mutation. Examples of activating AKT1 mutations include missense mutations resulting in the Q79K substitution. In one embodiment, the assaying for an activating mutation of AKT1 comprises measuring phospho-AKT1 levels.

The method can be performed prior to B-RAF inhibitor therapy, and/or after initiation of B-RAF inhibitor therapy. In some embodiments, the method is repeated during the course of treatment to monitor the status of resistance to B-RAF inhibitor therapy. In such embodiments, the same method steps are applied to a method of monitoring a patient being treated with B-RAF inhibitor therapy. In the course of such monitoring, the patient may be identified as a candidate for treatment with an alternative to B-RAF inhibitor therapy.

In one embodiment, the B-RAF inhibitor is vemurafenib. The sample obtained from the patient can be a biopsy or other clinical specimen obtained, for example, by needle aspiration or other means of extracting a specimen from the patient that contains tumor cells. The sample can also be obtained from peripheral blood or accessible bodily fluids, for example, by enriching a sample for circulating tumor cells. Examples of other accessible bodily fluids include, but are not limited to, the accumulation of peritoneal ascites, such as those caused by tumor deposits, and cerebrospinal fluid (CSF).

In some embodiments, the patient has, or is suspected of having, a B-RAF-mutant cancer. In a typical embodiment, the patient has, or is suspected of having, a B-RAF-mutant melanoma. A representative mutant B-RAF is ^{V600E}B-RAF.

Therapeutic and Prophylactic Methods

The invention further provides a method of treating a patient having cancer, or who may be at risk of developing cancer or a recurrence of cancer. In a typical embodiment, the patient has melanoma. In one embodiment, the melanoma is a B-RAF-mutant melanoma. The cancer can be melanoma or other cancer associated with B-RAF mutation, such as, for example, ^{V600E}B-RAF. Patients can be identified as candidates for treatment using the methods described herein. Patients are identified as candidates for treatment on the basis of exhibiting one or more indicators of resistance to B-RAF inhibitor therapy. The treatment protocol can be selected or modified on the basis of which indicators of resistance to B-RAF inhibitor therapy are exhibited by the individual patient.

The patient to be treated may have been initially treated with conventional B-RAF inhibitor therapy, or may be a patient about to begin B-RAF inhibitor therapy, as well as patients who have begun or have yet to begin other cancer treatments. Patients identified as candidates for treatment with one or more alternative therapies can be monitored so that the treatment plan is modified as needed to optimize efficacy.

Examples of alternative therapy include, but are not limited to, augmenting B-RAF inhibitor therapy with at least one additional drug. The additional drug can include a MAPK/ERK kinase (MEK) inhibitor, such as PD0325901, GDC0973, GSK1120212, and/or AZD6244. In one embodiment, the alternative therapy comprises suspension of vemurafenib therapy.

In one embodiment, the alternative therapy comprises administering to the patient a MEK inhibitor, optionally in conjunction with vemurafenib therapy, or an inhibitor of the MAPK pathway (RAF, MEK, ERK) in conjunction with an inhibitor of the RTK-PI3K-AKT-mTOR pathway. Examples of MEK inhibitors include, but are not limited to PD0325901, GDC0973, GSK1120212, and/or AZD6244§. Examples of inhibitors of the RTK-PI3K-AKT-mTOR pathway include, but are not limited to BEZ235, BKM120, PX-866, and GSK2126458.

Treatment includes prophylaxis and therapy. Prophylaxis or therapy can be accomplished by a single administration or direct injection, at a single time point or multiple time points to a single or multiple sites. Administration can also be nearly simultaneous to multiple sites. Patients or subjects include mammals, such as human, bovine, equine, canine, feline, porcine, and ovine animals. The subject is preferably a human. In a typical embodiment, treatment comprises administering to a subject a pharmaceutical composition of the invention.

A cancer may be diagnosed using criteria generally accepted in the art, including the presence of a malignant tumor. Pharmaceutical compositions may be administered either prior to or following surgical removal of primary tumors and/or treatment such as administration of radiotherapy or conventional chemotherapeutic drugs.

Administration and Dosage

The compositions are administered in any suitable manner, often with pharmaceutically acceptable carriers. Suitable methods of administering treatment in the context of the present invention to a subject are available, and, although more than one route can be used to administer a particular composition, a particular route can often provide a more immediate and more effective reaction than another route.

The dose administered to a patient, in the context of the present invention, should be sufficient to effect a beneficial therapeutic response in the patient over time, or to inhibit disease progression. Thus, the composition is administered to a subject in an amount sufficient to elicit an effective response and/or to alleviate, reduce, cure or at least partially arrest symptoms and/or complications from the disease. An amount adequate to accomplish this is defined as a “therapeutically effective dose.”

Routes and frequency of administration of the therapeutic compositions disclosed herein, as well as dosage, will vary from individual to individual as well as with the selected drug, and may be readily established using standard techniques. In general, the pharmaceutical compositions may be administered, by injection (e.g., intracutaneous, intratumoral, intramuscular, intravenous or subcutaneous), intranasally (e.g., by aspiration) or orally. In one example, between 1 and 10 doses may be administered over a 52 week period. Preferably, 6 doses are administered, at intervals of 1 month, and booster treatments may be given periodically thereafter. Alternate protocols may be appropriate for individual patients. In one embodiment, 2 intradermal injections of the composition are administered 10 days apart.

A suitable dose is an amount of a compound that, when administered as described above, is capable of promoting an anti-tumor immune response, and is at least 10-50% above the basal (i.e., untreated) level. Such response can be monitored using conventional methods. In general, for pharmaceutical compositions, the amount of each drug present in a dose ranges from about 100 μg to 5 mg per kg of host, but those skilled in the art will appreciate that specific doses depend on the drug to be administered and are not necessarily limited to this general range. Likewise, suitable volumes for each administration will vary with the size of the patient.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients.

### EXAMPLES

The following examples are presented to illustrate the present invention and to assist one of ordinary skill in making and using the same. The examples are not intended in any way to otherwise limit the scope of the invention.

### Example 1

### Melanomas Acquire Resistance to B-RAF(V600E) Inhibition by RTK or N-RAS Uprequlation

This example demonstrates that PLX4032 acquired resistance develops by mutually exclusive PDGFRβ (also known as PDGFRB) upregulation or N-RAS (also known as NRAS) mutations but not through secondary mutations in B-RAF(V600E). We used PLX4032-resistant sub-lines artificially derived from B-RAF(V600E)-positive melanoma cell lines and validated key findings in PLX4032-resistant tumours and tumour-matched, short-term cultures from clinical trial patients. Induction of PDGFRβ RNA, protein and tyrosine phosphorylation emerged as a dominant feature of acquired PLX4032 resistance in a subset of melanoma sub-lines, patient-derived biopsies and short-term cultures. PDGFRβ-upregulated tumour cells have low activated RAS levels and, when treated with PLX4032, do not reactivate the MAPK pathway significantly. In another subset, high levels of activated N-RAS resulting from mutations lead to significant MAPK pathway reactivation upon PLX4032 treatment. Knockdown of PDGFRβ or N-RAS reduced growth of the respective PLX4032-resistant subsets. Overexpression of PDGFRβ or N-RAS(Q61 K) conferred PLX4032 resistance to PLX4032-sensitive parental cell lines. Importantly, MAPK reactivation predicts MEK inhibitor sensitivity. Thus, melanomas escape B-RAF(V600E) targeting not through secondary B-RAF(V600E) mutations but via receptor tyrosine kinase (RTK)-mediated activation of alternative survival pathway(s) or activated RAS-mediated reactivation of the MAPK pathway, suggesting additional therapeutic strategies.

We selected three B-RAF(V600E)-positive parental (P) cell lines, M229, M238 and M249, exquisitely sensitive to PLX4032-mediated growth inhibition in vitro and in vivo⁶, and derived PLX4032-resistant (R) sub-lines by chronic PLX4032 exposure. In cell survival assays, M229 R, M238 R and M249 R sub-lines displayed strong resistance to PLX4032 (GI₅₀, concentration of drug that inhibits growth of cells by 50%,not reached up to 10 μM) and paradoxically enhanced growth at low PLX4032 concentrations, in contrast to parental cells. Morphologically, both M229 R and M238 R sub-lines appear flatter and more fibroblast-like compared to their parental counterparts, but this morphologic switch was not seen in the M249 P versus M249 R4 pair.

There were no secondary mutations in the drug target B-RAF observed on bi-directional Sanger sequencing of all 18 B-RAF exons in 15 M229 R (R1-R15), two M238 R (R1 and R2), and one M249 R (R4) acquired resistant sub-lines (Table 1). Based on Sanger sequencing, this lack of secondary B-RAF mutation along with retention of the original B-RAF(V600E) mutation was confirmed in 16/16 melanoma tumour biopsies (from 12 patients) with clinically acquired resistance to PLX4032 (that is, initial >30% tumour size decrease or partial response, as defined by RECIST (response evaluation criteria in solid tumours)) and subsequent progression on PLX4032 dosing; and 5/5 short-term melanoma cultures established from 5 resistant tumours obtained from 4 patients (Table 2). Given recent reports of B-RAF-selective inhibitors having a growth-promoting effect on B-RAF wild-type tumour cells⁷⁻⁹, retention of the original B-RAF alleles in PLX4032-resistant sub-lines, tissues and cultures indicates that PLX4032 chronic treatment did not select for the outgrowth of a pre-existing, minor B-RAF wild-type sub-population. Furthermore, immunoprecipitated B-RAF kinase activities from resistant sub-lines and short-term cultures were similarly sensitive to PLX4032 as B-RAF kinase activities immunoprecipitated from parental cell lines (Pt48 R and Pt55 R resistance to PLX4032 (ref. 10) and the pre-clinical analogue PLX4720 (ref. 11); Pt, patient). These results demonstrate that, in all tested acquired resistant cell lines and cultures, the mutated B-RAF(V600E) kinase lack secondary mutations and hence retain its ability to respond to PLX4032.

Given that minority PLX4032-resistant sub-populations in tissues may acquire B-RAF(V600E) secondary mutations not detectable by Sanger sequencing, we analysed “ultradeep” and deep sequences of B-RAF (exons 2-18) using the Illumina platform for 9/11 acquired resistant tumour samples without tumour-matched short-term cultures (one sample, Pt111-010 DP2, intentionally analysed by both methods; DP, disease progression). Ultradeep B-RAFsequencing of five PLX4032-resistant melanoma tissues resulted in every base of exons 2-18 being sequenced at a median coverage of 127×(27×−128×). The known variant, V600E, was detected in all five samples with significantly high non-reference allele frequencies (NAF). In all five tissues, exon 13, where the T529 gatekeeper residue¹² is located, was independently amplified and uniquely bar-coded twice. Rare variants (none at the T529 codon) detected in these independent exon 13 analyses do not overlap and helped defined the true, signal NAF at >4.81%. Furthermore, deep B-RAF (exons 2-18) sequence analysis of PLX4032-resistant melanoma tissues from a whole exome sequencing project resulted in 2,396 base pairs of B-RAF coding regions having coverage ≧10×. After filtering, no position harboured a variant with a NAF >4.81%, except for the known V600E mutation in all five resistant samples. Together, these data strongly corroborate the lack of B-RAF(V600E) secondary mutations during the evolution of PLX4032 acquired resistance in the majority of patients and their tumours.

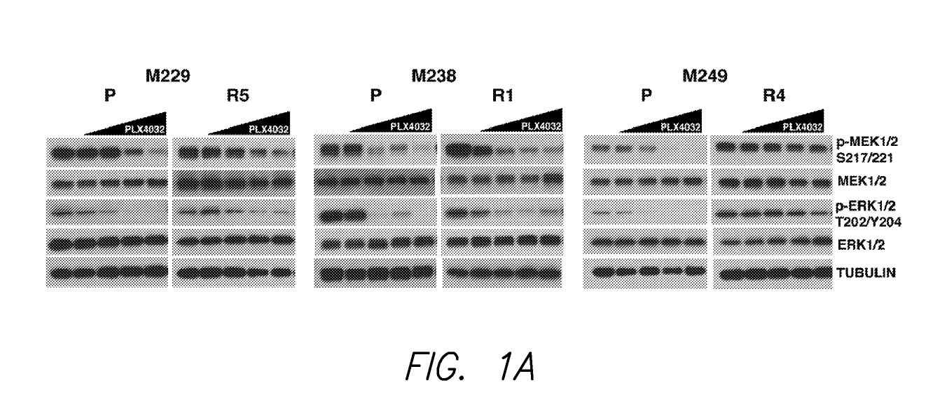

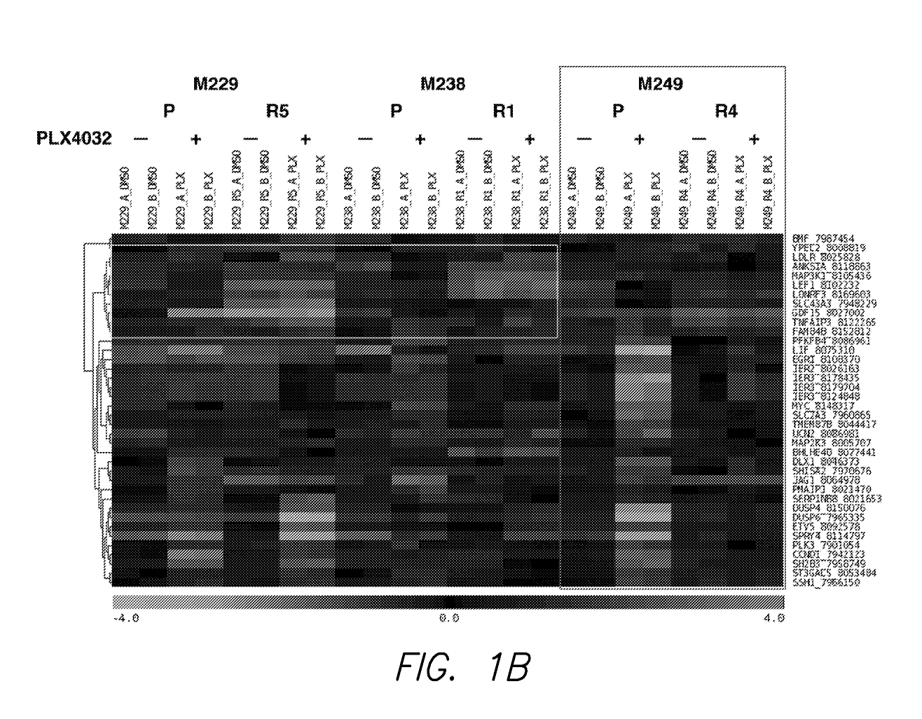

To begin to understand PLX4032-resistance in vitro, we used phospho-specific antibodies to probe the activation status of the RAF downstream effectors, MEK1/2 and ERK1/2 (also know as MAP2K1/2 and MAPK3/1, respectively), in parental versus resistant sub-lines, with and without PLX4032 (FIG. 1A). As expected, PLX4032 induced dose-dependent decreases in p-MEK1/2 and p-ERK1/2 in all parental cells. However, the pattern of MEK-ERK sensitivity to PLX4032 varied among resistant sub-lines, suggesting distinct mechanisms. In contrast to M249 R4, which showed strong resistance to PLX4032-induced MEK/ERK inhibition (suggesting MAPK reactivation), M229 R5 and M238 R1 were both similarly sensitive to PLX4032-induced decreases in the levels of p-MEK1/2 and p-ERK1/2. Gene expression profiling (FIG. 1B) further supported distinct PLX4032 acquired resistant mechanisms represented by M229 R5/M238 R1 versus M249 R4. We first used the gene expression alterations responsive to PLX4032 in parental cells to define a B-RAF(V600E)-responsive gene signature, which is similar to gene sets defined by a MEK1 inhibitor (PD325901)¹³ and by PLX4720 (ref. 14). Concordant with the western blot results (FIG. 1A), M249 R4 demonstrated striking resistance to PLX4032 treatment with a gene signature of persistent MEK-ERK activation, whereas both M229 R5 and M238 R1 retained a PLX4032-sensitive gene signature (FIG. 1B). These data confirm that M229 R5 and M238 R1 share key characteristics of resistance, which are in line with unsupervised clustering of these two resistant sub-lines in genome-wide, differential expression patterns.

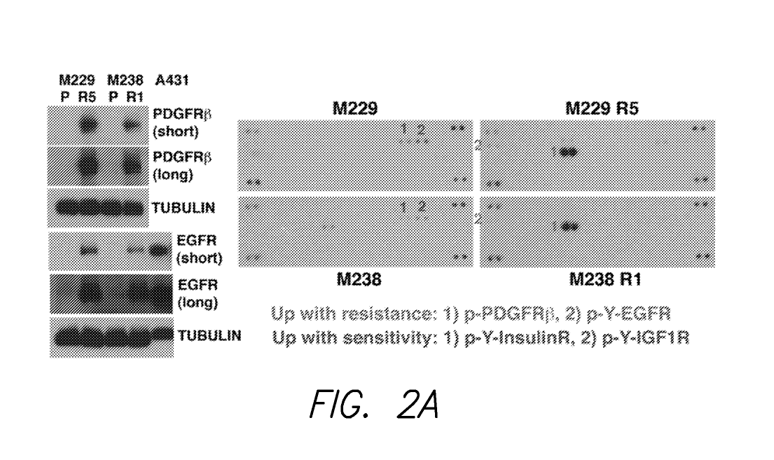

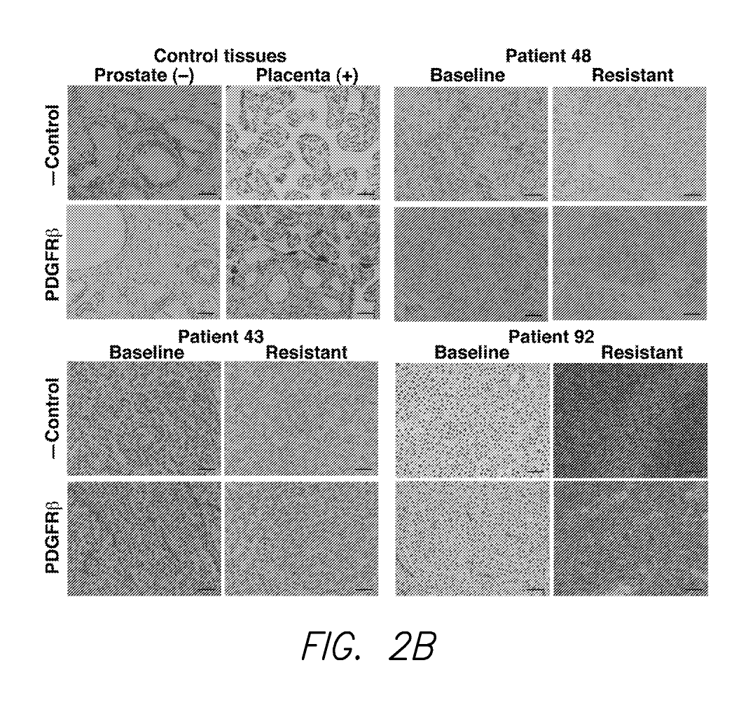

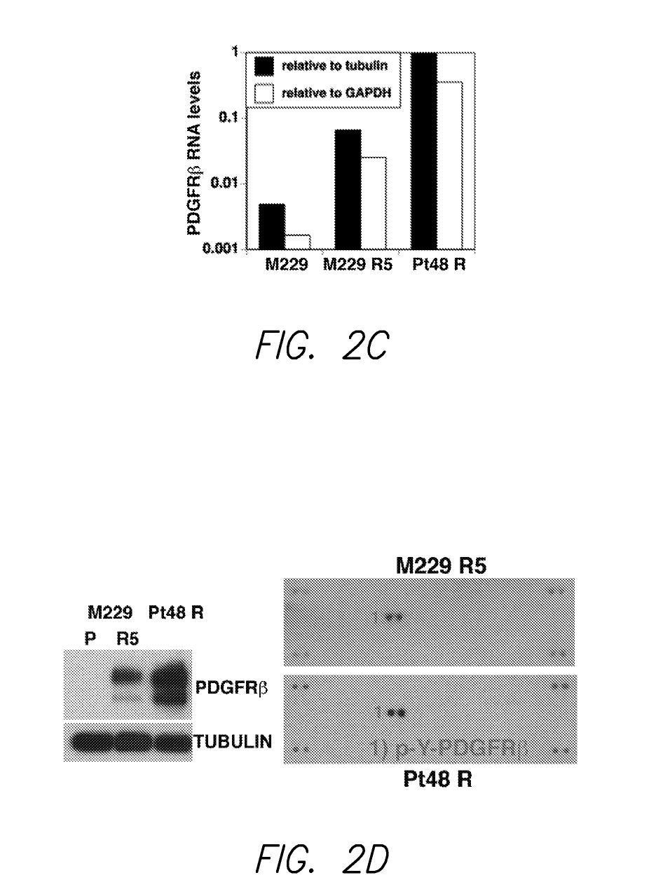

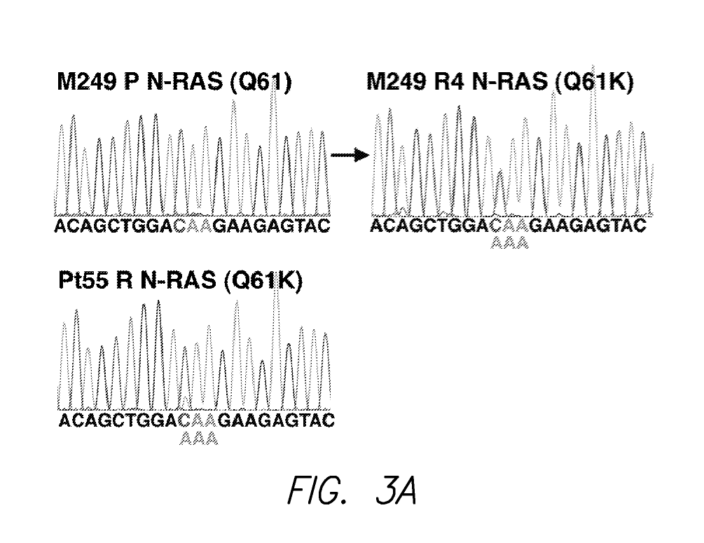

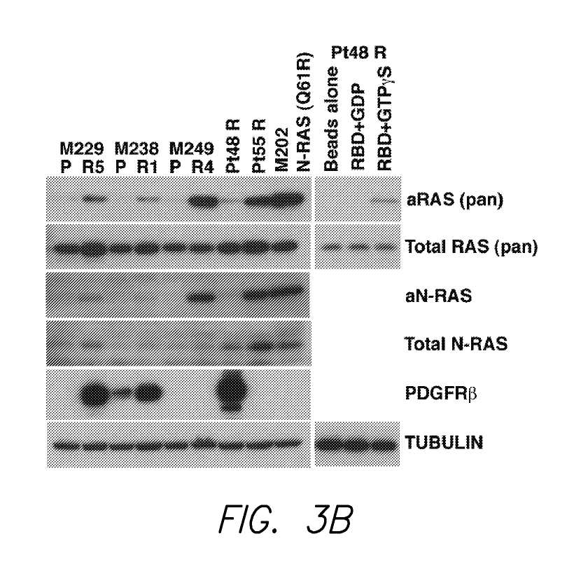

Gene set enrichment analysis demonstrated an enrichment of RTK-controlled signalling in M229 R5 and M238 R1 but exclusive of M249 R4. Unsupervised clustering of the receptor tyrosine kinome gene expression profiles showed that M229 R5 and M238 R1 clustered away from M229 and M238 parental cell lines largely based on higher expression levels of KIT, MET, EGFR and PDGFRβ. RNA upregulation of these four RTKs was consistently not associated with genomic DNA (gDNA) copy number gain. Of these four candidate RTKs, EGFR and PDGFR6 protein levels were overexpressed (FIG. 2A, left; FIG. 3B), but only PDGFRβ displayed elevated activation-associated tyrosine phosphorylation in a phospho-RTK array (FIG. 2A, right). PDGFRβ RNA upregulation was a common feature among additional M229 R and M238 R sub-lines but could not be observed in any of ten randomly selected parental melanoma cell lines. Interestingly, tyrosine phosphorylation of PDGFR correlated with an upregulation of a gene signature unique to PDGFRβ (ref. 15) but is not due to mutational activation, as PDGFRβ cDNAs derived from M229 R5, M238 R1 and Pt48 R are wild type (Table 1).

TABLE 1
Sequencing primers.

| B-RAF | Forward (SEQ ID NOs: 2-19) | Reverse (SEQ ID NOs: 20-37) |
|---|---|---|
| Exon1 | CGGCGACTTCTCGTCGTCTC | CTGCATGACGGAGAGGGACA |
| Exon2 | CTGGCAGTTACTGTGATGTAGTTG | CTTCCCAAATCTATTCCTAATCCCACC |
| Exon3 | GGACCATCTAGATATCACATATG | CATTCCTGTATGACATGGATGCCTC |
| Exon4 | GTAGAAATGGTGTTGTATCTGACC | GATCAAAGTAACAAACCCTACAGTC |
| Exon5 | CGATGGAATATTAGGGAGCCAAACC | CTAAACAAATGTTGGCCTCTAGG |
| Exon6 | GTTGTCAAGCTTGAAATCAGTTGC | CTGTATAGCTGAACCAGCATTAC |
| Exon7 | CTGAGAATGGAATTTGATCTC | GCATGTCACTGAAGAGCAGAAGTC |
| Exon8 | CTTGAGCAAAGCAGCTTTGGC | CAGAAGCTTTTCTGATTTGTGATTC |
| Exon9 | GTGTCCACTTGTTCTTATCATTCAGC | GTTTCTCTACACATTTTTCTCTGTG |
| Exon10 | GTATGTGTCTATGTCTATCATC | GAATTCTGTGTCACATATGGAC |
| Exon11 | CTCTTCCTGTATCCCTCTCAGGC | GGTAGGAGTCCCGACTGCTGTGAAC |
| Exon12 | CTAGTACAGGAATATCATTGTTAG | CTATCAGCCATACCATATAACATTGC |
| Exon13 | GTAGGAGGTTAGACTTGGCAATTGC | GTTAGCATCCTTATGTTCCTGGAC |
| Exon14 | CTTGACTGGAGTGAAAGGTTTG | CAGGCTGTGGTATCCTGCTCTCC |
| Exon15 | GACTCTAAGAGGAAAGATGAAGTAC | GTTGAGACCTTCAATGACTTTCTAG |
| Exon16 | CTGTCTCTTTCTGAGTATGTAG | CTATCCTTCACGCTTACCCAGGAG |
| Exon17 | GTGGGTTTCCCACCATCTATGATG | GAGTCTGCACATAGAATCCAAACTC |
| Exon18 | GATTTCAGGTGCTTTCTTGTAAAGTG | CCACACAAGTGTTCTTTGGTTC |
| PDGFRβ | Forward (SEQ ID NOs: 38-43) | Reverse (SEQ ID NOs: 44-49) |
| Primer 1 | AGAGGGCAGTAAGGAGGACTTCC | ACCTCCCTGTCCCCAATGGTGG |
| Primer 2 | ACAGACCCACAGCTGGTGGTG | TCTGCCACCTTCACGCGAACC |
| Primer 3 | ACCGCCCACTGTCCTGTGGTTC | AGTCATAGGGCAGCTGCATGGG |
| Primer 4 | TGATCTCAGCCATCCTGGCCC | TAGTTGGAGGACTCGATGTCTGC |
| Primer 5 | ATGTGTCCTTGACCGGGGAGAG | AGTCTCTCGAGAAGCAGCACCAG |
| Primer 6 | TACCCAGAGCTGCCCATGAACG | AGAAGGGGACAGCTGATAAGGGC |
| N-RAS | Forward (SEQ ID NOs: 50-53) | Reverse (SEQ ID NOs: 54-57) |
| Exon1 | TAAAGTACTGTAGATGTGGCTCGCC | ACAGAATATGGGTAAAGATGATCCGAC |
| Exon2 | GGCTTGAATAGTTAGATGCTTATTTAACCTTGGC | GCTCTATCTTCCCTAGTGTGGTAACCTC |
| Exon3 | CCACTGTACCCAGCCTAATC | AAGAGACAGAGGCTGCAGTG |
| Exon4 | ACACCAGCCCGTTTATGGCT | TGTGCAGAAGAGGATAGGCAGA |
| K-RAS | Forward (SEQ ID NOs: 58-61) | Reverse (SEQ ID NOs: 62-65) |
| Exon1 | AAGGTACTGGTGGAGTATTTG | GTACTCATGAAAATGGTCAGAG |
| Exon2 | TGAAGTAAAAGGTGCACTGTAATAATCCAG | CATTTATAAAACAGGGATATTACCTACCTC |
| Exon3 | TGACAAAAGTTGTGGACAGGT | GCAATGCCCTCTCAAGAGACAA |
| Exon4 | ACAAAACACCTATGCGGATGACA | AACAGTCTGCATGGAGCAGG |
| H-RAS | Forward (SEQ ID NOs: 66-69) | Reverse (SEQ ID NOs: 70-73) |
| Exon1 | GGCTGAGCAGGGCCCTCCTTGGCAGG | GCCCTATCCTGGCTGTGTCCTGGGC |
| Exon2 | GGTACCAGGGAGAGGCTGGCTGTGTGAAC | CAGCGGCATCCAGGACATGCGCAG |
| Exon3 | TACAGGTGAACCCCGTGAGG | GGAGAGGGTCAGTGAGTGCT |
| Exon4 | ACCTTTGAGGGGCTGCTGTA | CACAAGGGAGGCTGCTGAC |
| MEK1 | Forward (SEQ ID NOs: 74-75) | Reverse (SEQ ID NOs: 76-77) |
| Exon2 | GCTTTCTTTCCATGATAGGAGTAC | ATCAGTCTTCCTTCTACCCTGG |
| Exon3 | CCTGTTTCTCCTCCCTCTACC | ACACCCACCAGGAATACTGC |

We then validated our in vitro finding in vivo by studying clinical trial patient-derived samples (Table 2; FIG. 2B) and tumour-matched short-term cultures (FIGS. 2C and D). In 4/11 available, paired biopsy specimens, the resistant tumours showed a tumour-associated overexpression of PDGFRβ compared to the baseline tumour in the same patients (FIG. 2B; Table 2). PDGFRβ-positive areas of tissue sections were consistently strongly positive for S100 or MART1 (melanoma markers; MART1 is also known as MLANA) but lacked CD31 (an endothelial, platelet, macrophage marker, also known as PECAM1) staining. We were able to validate this finding further in an available short-term culture (Pt48 R) derived from a PLX4032-resistant, PDGFRβ-positive tumour. Pt48 R was established from an intracardiac mass progressing 6 months after initiating treatment with PLX4032. The Pt48 R short-term culture demonstrated clear overexpression of PDGFRβ RNA (FIG. 20), protein and p-Tyr levels (FIG. 2D).

TABLE 2
Patient characteristics, available patient-matched tumor samples and tumor-matched
short-term cultures, and summary of B-RAF/RAS sequencing and PDGFRβ expression.

| | | | | Best | Progression | | | B-RAF | | | | | |
|---|---|---|---|---|---|---|---|---|---|---|---|---|---|
| Patient | | | | overall | free survival | Biopsy & | Site of | exons 1-14, | B-RAF | | | | |
| ID | Sex | Age | Stage | response | (days) | culture ID | biopsy | 16-18 | exon 15 | N-RAS | K-RAS | H-RAS | PDGFRβ |
| Pt23 | M | 62 | M1a | 57% | 466 | Pt23 | SC mass- | N/D | V600E | N/D | N/D | N/D | − |
| | | | | | | baseline | thigh | | | | | | |
| | | | | | | Pt23 | Large | WT\hat{ } | V600E | WT | WT | WT | + |
| | | | | | | resistant | bowel | | | | | | |
| | | | | | | | (cecum) | | | | | | |
| Pt43 | M | 52 | M1c | 83% | 161 | Pt43 | Pelvic | N/D | V600E | N/D | N/D | N/D | − |
| | | | | | | baseline | bone | | | | | | |
| | | | | | | | metastasis | | | | | | |
| | | | | | | Pt43 | Bowel | WT\hat{ } | V600E | WT | WT | WT | − |
| | | | | | | resistant | | | | | | | |
| Pt48 | M | 30 | M1b | 14% | 113 | Pt48 | SC mass, | N/D | V600E | N/D | N/D | N/D | − |
| | | | | | | baseline | shoulder | | | | | | |
| | | | | | | Pt48 | Heart | WT | V600E | WT | WT | WT | + |
| | | | | | | resistant | | | | | | | |
| | | | | | | Pt48 R | Heart | WT | V600E | WT | WT | WT | + |
| Pt55 | F | 65 | M1c | 37% | 270 | Pt55 | Femoral | N/D | V600E | WT | N/D | N/D | − |
| | | | | | | baseline | node | | | | | | |
| | | | | | | Pt55 | Inguinal | WT | V600E | Q61K | WT | WT | − |
| | | | | | | resistant | node* | | | | | | |
| | | | | | | DP1 | | | | | | | |
| | | | | | | Pt55 R | Inguinal | WT | V600E | Q61K | WT | WT | − |
| | | | | | | | node* | | | | | | |
| | | | | | | Pt55 | Small | WT | V600E | Q61R | WT | WT | N/D |
| | | | | | | resistant | bowel | | | | | | |
| | | | | | | DP2 | | | | | | | |
| | | | | | | Pt55 | Small | WT | V600E | Q61R | WT | WT | N/D |
| | | | | | | R2 | bowel | | | | | | |
| Pt56 | F | 45 | M1c | 53% | 106 | Pt56 | Right | N/D | V600E | N/D | N/D | N/D | − |
| | | | | | | baseline | pubic | | | | | | |
| | | | | | | | area | | | | | | |
| | | | | | | Pt56 | Right | WT\hat{ } | V600E | WT | WT | WT | − |
| | | | | | | resistant | pubic | | | | | | |
| | | | | | | | (labial) | | | | | | |
| Pt81 | M | 44 | M1a | 73% | 141 | Pt81 | SC | N/D | V600E | N/D | N/D | N/D | − |
| | | | | | | baseline | | | | | | | |
| | | | | | | Pt81 | Small | WT\hat{ } | V600E | WT | WT | WT | − |
| | | | | | | resistant | bowel | | | | | | |
| Pt84 | M | 60 | M1c | 84% | 113 | Pt84 | Cutaneous | N/D | V600E | N/D | N/D | N/D | − |
| | | | | | | baseline | | | | | | | |
| | | | | | | Pt84 | Cutaneous | WT | V600E | WT | WT | WT | − |
| | | | | | | resistant | | | | | | | |
| Pt92 | M | 46 | M1a | 32% | 149 | Pt92 | Axillary | N/D | V600E | N/D | N/D | N/D | − |
| | | | | | | day 15 | node | | | | | | |
| | | | | | | Pt92 | Abdominal | WT | V600E | WT | WT | WT | + |
| | | | | | | resistant | mass | | | | | | |
| Pt111- | F | 66 | M1b | 53% | 137 | Pt111- | Cutaneous, | N/D | V600E | WT | WT | WT | − |
| 001 | | | | | | 001 | left | | | | | | |
| | | | | | | baseline | clavicular** | | | | | | |
| | | | | | | Pt111- | Cutaneous, | WT# | V600E | WT | WT | WT | − |
| | | | | | | 001 | left | | | | | | |
| | | | | | | resistant | lateral | | | | | | |
| | | | | | | DP1 | neck** | | | | | | |
| | | | | | | Pt111- | Cutaneous, | WT# | V600E | WT | WT | WT | − |
| | | | | | | 001 | left | | | | | | |
| | | | | | | resistant | clavicular** | | | | | | |
| | | | | | | DP2 | | | | | | | |
| | | | | | | Pt111- | Cutaneous, | WT# | V600E | WT | WT | WT | − |
| | | | | | | 001 | left | | | | | | |
| | | | | | | resistant | shoulder** | | | | | | |
| | | | | | | DP3 | | | | | | | |
| Pt111- | M | 72 | M1c | 42% | 126 | Pt111- | N/A | N/A | N/A | N/A | N/A | N/A | N/A |
| 005 | | | | | | 005 | | | | | | | |
| | | | | | | baseline | | | | | | | |
| | | | | | | Pt111- | Adrenal | WT | V600E | WT | WT | WT | N/D |
| | | | | | | 005 | gland | | | | | | |
| | | | | | | resistant | | | | | | | |
| | | | | | | Pt111- | Adrenal | WT | V600E | WT | WT | WT | − |
| | | | | | | 001 R | gland | | | | | | |
| Pt111- | M | 48 | M1c | 24% | 126 | Pt111- | Lymph | N/D | V600E | N/D | N/D | N/D | − |
| 010 | | | | | | 010 | node, left | | | | | | |
| | | | | | | baseline | inguinal | | | | | | |
| | | | | | | Pt111- | Cutaneous, | WT# | V600E | WT | WT | WT | N/D |
| | | | | | | 010 | left | | | | | | |
| | | | | | | resistant | anterior | | | | | | |
| | | | | | | DP1 | thigh, | | | | | | |
| | | | | | | | superior | | | | | | |
| | | | | | | Pt111- | Cutaneous, | WT\hat{ }# | V600E | WT | WT | WT | − |
| | | | | | | 010 | left | | | | | | |
| | | | | | | resistant | anterior | | | | | | |
| | | | | | | DP2 | thigh, | | | | | | |
| | | | | | | | inferior | | | | | | |
| | | | | | | Pt111- | Cutaneous, | WT | V600E | WT | WT | WT | − |
| | | | | | | 010 R | left | | | | | | |
| | | | | | | | anterior | | | | | | |
| | | | | | | | thigh, | | | | | | |
| | | | | | | | inferior | | | | | | |
| Pt104- | M | 54 | M1c | 75% | 84 | Pt104- | Lung | N/D | V600E | N/D | N/D | N/D | − |
| 004 | | | | | | 004 | | | | | | | |
| | | | | | | baseline | | | | | | | |
| | | | | | | Pt104- | Pelvic | WT | V600E | WT | WT | WT | + |
| | | | | | | 004 | mass | | | | | | |
| | | | | | | resistant | | | | | | | |

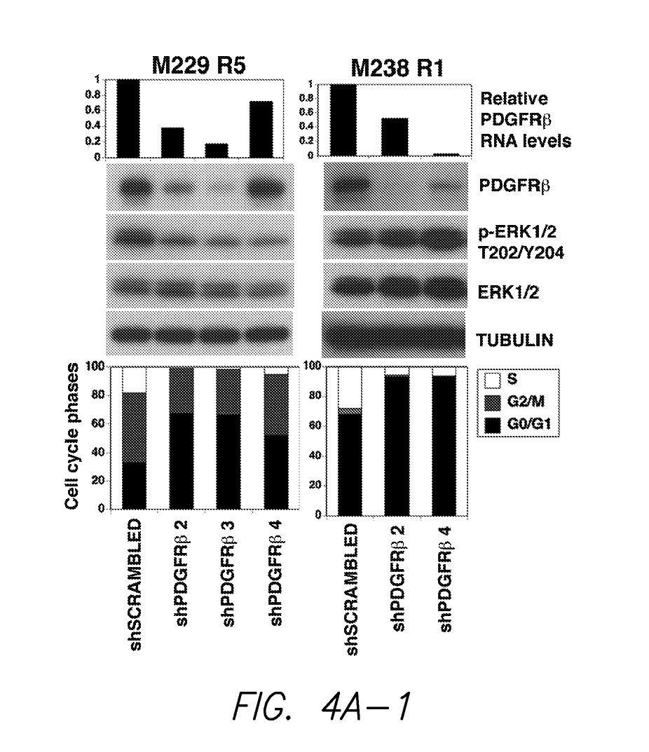

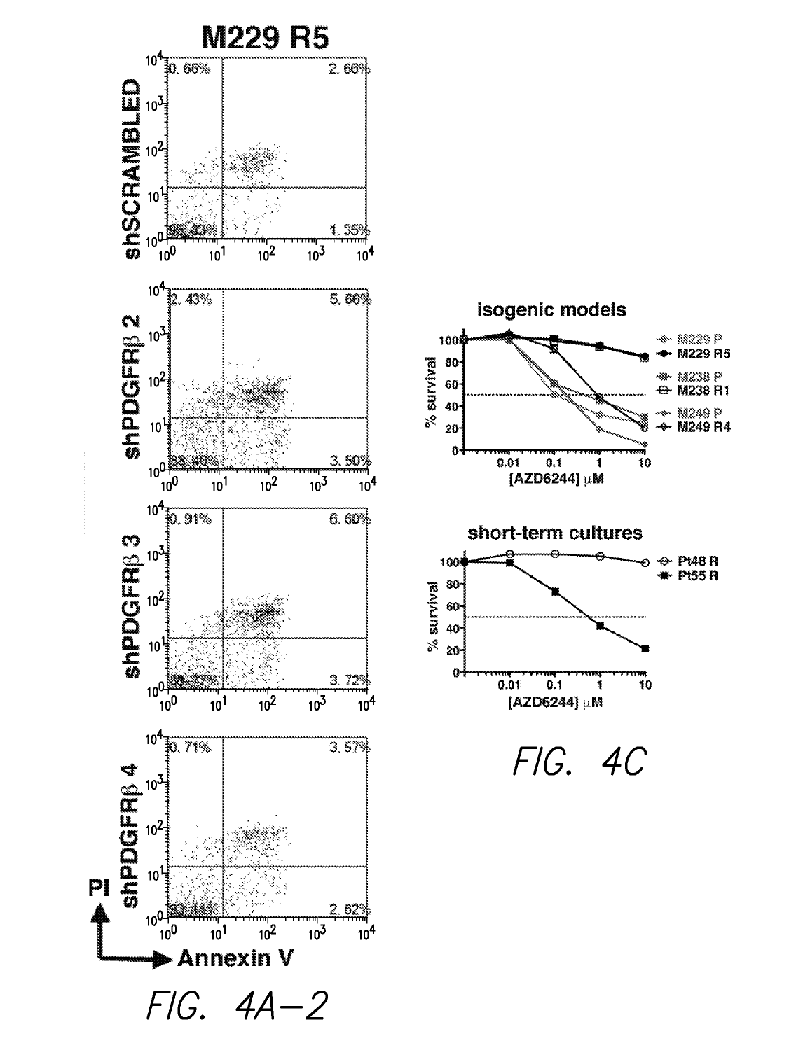

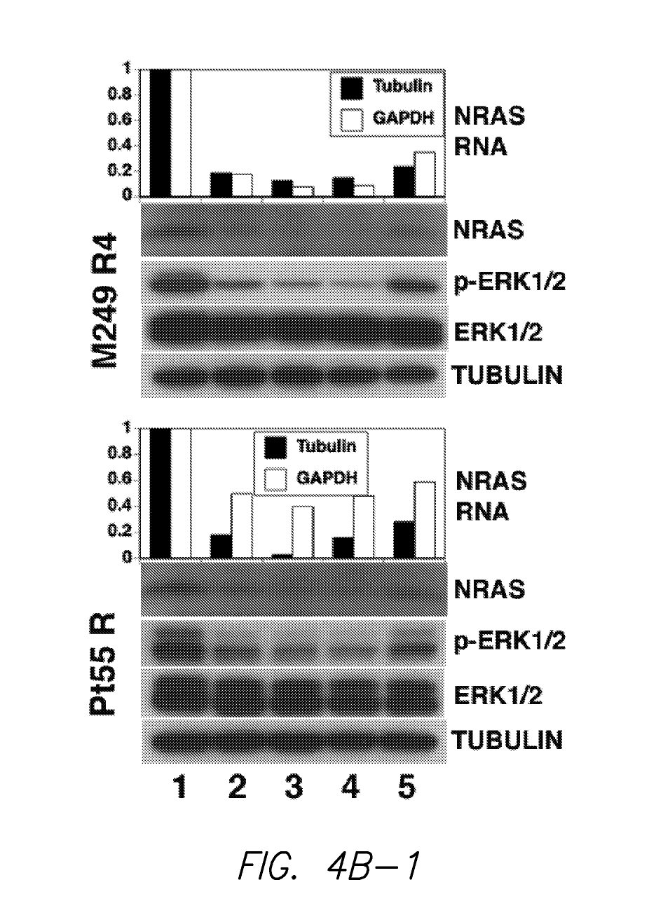

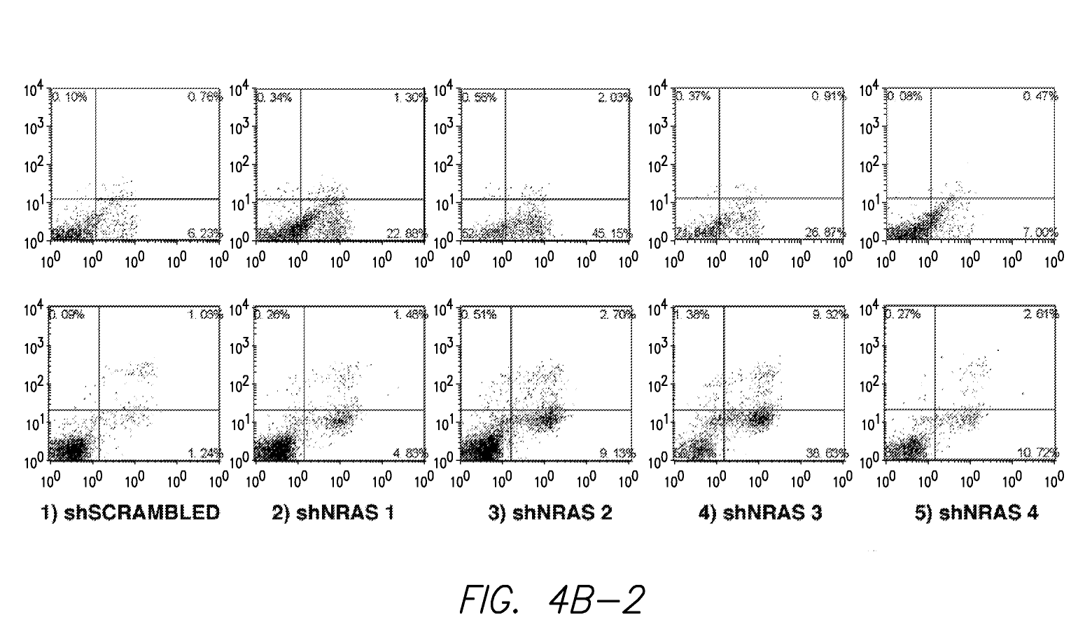

Mutation status of B-RAF and RAS genes and PDGFRβ expression status are summarized for a collection of PLX4032 clinical trial biopsy samples and tumor-matched short-term cultures. Samples labeled as resistant are from tumors that initially responded to (PR 30% by RECIST criteria) and then progressed on PLX4032.
Abbreviations and symbols: M, male; F, female; SC, subcutaneous; Pt# R, short-term culture derived from tissue directly above; DP, disease progression; N/D, not done; N/A, not available;
*shown in Suppl. FIG. 4a;
**shown in Suppl. FIG. 4b;
\hat{ }shown by ultradeep sequencing in addition to Sanger sequencing;
#shown by deep sequencing as well as Sanger sequencing.
PDGFRβ expression determined by immunohistochemistry (IHC) for tissues and immunoblotting for short-term cultures (relative to PLX4032-sensitive parental and PLX4032-resistant sub-lines). PDGFRβ IHC is performed only for the available elevan, baseline/resistant, patient-paired, tumor samples and defined as positive if specific immuno-reactivity exceeds 20% within representative tumor sections.

In M249 R4, we sequenced all exons of N-RAS, K-RAS (also known as KRAS) or H-RAS (also known as HRAS) (to include codons 12, 13, and 61 as well as mutational hotspots of emerging significance¹⁶) and MEK1 (ref. 17; Table 1) because we proposed a resistance mechanism reactivating MAPK despite not having a secondary B-RAF mutation. Interestingly, M249 R4 harbours a N-RAS(Q61K) activating mutation not present in the parental M249 cell line (FIG. 3A). We found N-RAS mutations in 2/16 acquired resistant biopsy samples (note that both came from Pt55; Table 2). A N-RAS(Q61K) mutated sample, Pt55 DP1 (for disease progression 1) was obtained from a biopsy taken from an isolated, nodal metastasis that partially regressed on PLX4032 but increased in size 10 months after starting on therapy with PLX4032. This patient continued on therapy with PLX4032 until 6 months later, when several other nodal metastases developed. Analysis of a biopsy taken at a second progression site (Pt55 DP2) demonstrated a different mutation in N-RAS, N-RAS(Q61R). Both Pt55 DP1 and DP2 tissue N-RAS mutations were confirmed in their respective short-term cultures, Pt55 R and Pt55 R2 (FIG. 3A). Also, both DP1 and DP2 (and their respective cultures) harboured increased N-RAS gDNA copy numbers. Both Pt55 R and Pt55 R2 also showed increased N-RAS RNA and protein levels (FIG. 3B). In addition, N-RAS(Q61K) mutation in M249 R4 and Pt55 R correlated with a marked increase in activated N-RAS levels (FIG. 3B). Of note, the N-RAS mutations were mutually exclusive with PDGFRβ overexpression in all samples (Table 2).

Knockdown of PDGFRβ or N-RAS using small interfering RNA (siRNA) pools preferentially growth-inhibited melanoma cells with upregulated PDGFRβ or N-RAS, respectively (Table 3). We then selected two resistant sub-lines or cultures to test the effects of individual PDGFRβ and N-RAS short hairpin RNAs (shRNAs; FIGS. 4A and B, respectively). Stable knockdown of PDGFRβ caused an admixture of G0/G1 cell cycle arrest (in a MEK inhibitor-dependent manner due to compensatory signalling) and apoptosis in M229 R5 and a G0/G1 cell cycle arrest in M238 R1. This effect was specific, as stable PDGFRβ knockdown in M249 R4 and Pt55 R did not result in G0/G1 cell cycle arrest. In contrast, stable N-RAS knockdown resulted in a predominantly apoptotic response in M249 R4 and Pt55 R (FIG. 4B) but not in M229 R5, M238 R1 or Pt48 R. Moreover, stable N-RAS knockdown markedly conferred PLX4032 sensitivity to M249 R4 and Pt55 R but had not effect on M229 R5 PLX4032 resistance. Flag-N-RAS(Q61 K) stable overexpression conferred PLX4032 resistance in the M249 parental cell line, whereas stable PDGFRβ-MYC overexpression conferred reduced PLX4032 sensitivity in both M229 and M238 parental cell lines.

TABLE 3
shRNA sequences based on siRNA sequences (SEQ ID NOs: 78-93)

| shRNA ID | Oligonucleotide sequence |
|---|---|
| shRNACONTROL | TGGAATCTCATTCGATGCATACTTCAAGAGAGTATGCATCGAATGAGATTCCTTTTTTC |
| (sense) | |
| shRNACONTROL | TCGAGAAAAAAGGAATCTCATTCGATGCATACTCTCTTGAAGTATGCATCGAATGAGATTCCA |
| (antisense) | |
| shPDGFRβ2 (sense) | TGAGCGACGGTGGCTACATGTTCAAGAGACATGTAGCCACCGTCGCTCTTTTTTC |
| shPDGFRβ2 | TCGAGAAAAAAGAGCGACGGTGGCTACATGTCTCTTGAACATGTAGCCACCGTCGCTCA |
| (antisense) | |
| shPDGFRβ3 (sense) | TGAAGCCACGTTACGAGATCTTCAAGAGAGATCTCGTAACGTGGCTTCTTTTTTC |
| shPDGFRβ3 | TCGAGAAAAAAGAAGCCACGTTACGAGATCTCTCTTGAAGATCTCGTAACGTGGCTTCA |
| (antisense) | |
| shPDGFRβ4(sense) | TGGTGGGCACACTACAATTTCCACACCAAATTGTAGTGTGCCCACCTTTTTTC |
| shPDGFRβ4 | TCGAGAAAAAAGGTGGGCACACTACAATTTGGTGTGGAAATTGTAGTGTGCCCACCA |
| (antisense) | |
| shNRAS1 (sense) | TGAGCAGATTAAGCGAGTAATTCAAGAGATTACTCGCTTAATCTGCTCTTTTTTC |
| shNRAS1 (antisense) | TCGAGAAAAAAGAGCAGATTAAGCGAGTAATCTCTTGAATTACTCGCTTAATCTGCTCA |
| shNRAS2 (sense) | TGAAATACGCCAGTACCGAATTCAAGAGATTCGGTACTGGCGTATTTCTTTTTTC |
| shNRAS2 (antisense) | TCGAGAAAAAAGAAATACGCCAGTACCGAATCTCTTGAATTCGGTACTGGCGTATTTCA |
| shNRAS3 (sense) | TGTGGTGATGTAACAAGATATTCAAGAGATATCTTGTTACATCACCACTTTTTTC |
| shNRAS3 (antisense) | TCGAGAAAAAAGTGGTGATGTAACAAGATATCTCTTGAATATCTTGTTACATCACCACA |
| shNRAS4 (sense) | TGCACTGACAATCCAGCTAATTCAAGAGATTAGCTGGATTGTCAGTGCTTTTTTC |
| shNRAS4 (antisense) | TCGAGAAAAAAGCACTGACAATCCAGCTAATCTCTTGAATTAGCTGGATTGTCAGTGCA |

We then asked whether N-RAS-dependent growth and reactivation of the MAPK pathway (FIG. 1A) would selectively sensitize M249 R4 and Pt55 R to MEK inhibition. Indeed, whereas the growth of M229 R5, M238 R1 and Pt48 R was uniformly highly resistant to the MEK inhibitor AZD6244 (and U0126), the growth of M249 R4 and Pt55 R was sensitive to MEK inhibition in the presence of PLX4032 (FIG. 4C) or absence of PLX4032. It is known that activated N-RAS in melanoma cells uses C-RAF (also known as RAF1) over B-RAF to signal to MEK-ERK¹⁸. Thus, N-RAS activation would be capable of bypassing PLX4032-inhibited B-RAF, reactivating the MAPK pathway. It is worth noting that PDGFRβ-upregulated, PLX4032-resistant melanoma sub-lines (M229 R5 and M238 R1) and culture (Pt48 R) are resistant not only to AZD6244 but also to imatinib, which is at least partially due to rebound, compensatory survival signalling.

We show that B-RAF(V600E)-positive melanomas, instead of accumulating B-RAF(V600E) secondary mutations, can acquire PLX4032 resistance by (1) activating an RTK (PDGFRβ)-dependent survival pathway in addition to MAPK, or (2) reactivating the MAPK pathway via N-RAS upregulation. These two mechanisms account for acquired PLX4032 resistance in 5/12 patients in our study cohort, and additional mechanisms await future discovery. Some patients who relapse on PLX4032 are already being enrolled in a phase II MEK inhibitor trial (ClinicalTrials.gov identifier NCT01037127) based on the assumption of MAPK reactivation. Our findings provide a strategy to stratify patients who relapse on PLX4032 and rational combinations of targeting agents most optimal for distinct mechanisms of acquired resistance to PLX4032 as well as other B-RAF inhibitors (for example, GSK2118436) in clinical development.

Methods Summary

Cell culture, infections and compounds

Cells were maintained in Dulbecco's modified Eagle medium (DMEM) with 10 or 20% fetal bovine serum and glutamine. shRNAs were sub-cloned into the lentiviral vector pLL3.7 and infections carried out with protamine sulphate. Stocks of PLX4032 (Plexxikon) and AZD6244 (commercially available) were made in DMSO. Cells were quantified using CellTiter-GLO Luminescence (Promega).

Protein Detection

Western blots were probed with antibodies against p-MEK1/2 (S217/221), MEK1/2, p-ERK1/2 (T202N204), ERK1/2, PDGFRβ, and EGFR (Cell Signaling Technologies), and N-RAS (Santa Cruz Biotechnology), pan-RAS (Thermo Scientific) and tubulin (Sigma). p-RTK arrays were performed according to the manufacturer's recommendations (Human Phospho-RTK Array Kit, R&D Systems). For PDGFRβ immunohistochemistry, paraffin-embedded formalin-fixed tissue sections were antigen-retrieved, incubated with a PDGFRβ antibody followed by horseradish peroxidase-conjugated secondary antibody (Envision System, DakoCytomation). Immunocomplexes were visualized using the DAB (3,3′-diaminobenzidine) peroxidase method and nuclei haematoxylin-counterstained. For activated RAS pull-down, lysates were incubated with beads coupled to glutathione-S-transferase (GST)-RAF-1-RAS-binding domain of RAF1 (RBD) (Thermo) for 1 h at 4° C.

RNA Quantifications

For real-time quantitative PCR, total RNA was extracted and cDNA quantified. Data were normalized to tubulin and GAPDH levels. Relative expression is calculated using the delta-Ct method. For RNA expression profiling, total RNAs were extracted, and generated cDNAs were fragmented, labelled and hybridized to the GeneChip Human Gene 1.0 ST Arrays (Affymetrix). Expression data were normalized, background-corrected, and logₑ-transformed for parametric analysis. Differentially expressed genes were identified using significance analysis of microarrays (SAM) with the R package ‘samr’ (false discovery rate (FDR)<0.05; fold change>2).

Cell Cycle and Apoptosis

For cell cycle analysis, cells were fixed, permeabilized and stained with propidium iodide (BD Pharmingen). Cell cycle distribution was analysed by Cell Quest Pro and ModiFit software. For apoptosis, cells were co-stained with Annexin V-V450 and propidium iodide (BD Pharmingen). Data were analysed with the FACS Express V2 software.

Methods

Cell Culture, Lentiviral Constructs and Infections

All cell lines were maintained in DMEM with 10% or 20% (short-term cultures) heat-inactivated FBS (Omega Scientific) and 2 mmol l⁻¹ glutamine in humidified, 5% CO₂ incubator. To derive PLX4032-resistant sub-lines, M229 and M238 were seeded at low cell density and treated with PLX4032 at 1 μM every 3 days for 4-6 weeks and clonal colonies were then isolated by cylinders. M249 R was derived by successive titration of PLX4032 up to 10 μM. PLX4032-resistant sub-lines and short-term cultures were replenished with 1 μM PLX4032 every 2 to 3 days. shRNAs were sub-cloned into the lentiviral vector pLL3.7. N-RAS(Q61K) mutant overexpression construct was made by PCR-amplifying from M249 R4 cDNA and sub-cloning into the lentiviral vector (UCLA Vector Core), creating pRRLsin.cPPT.CMV.hTERT.IRES.GFP-Flag-^{Q61K}NRAS. Wild-type PDGFRβ overexpression construct was PCR-amplified from cDNA and sub-cloned into a lentiviral vector (Clontech), creating pLVX-Tight-Puro-PDGFRβ-Myc. Lentiviral constructs were co-transfected with three packaging plasm ids into HEK293T cells. Infections were carried out with protamine sulphate.

Cellular Proliferation, Drug Treatments and siRNA Transfections

Cell proliferation experiments were performed in a 96-well format (five replicates), and baseline quantification performed at 24 h after cell seeding along with initiation of drug treatments (72 h). Stocks and dilutions of PLX4032 (Plexxikon), AZD6244 (Selleck Chemicals) and U0126 (Promega) were made in DMSO. siRNA pool (Dharmacon) transfections were carried out in 384-well format. TransIT transfection reagent (Mirus) was added to each well and incubated at 37° C. for 20 min. Subsequently, cells were reverse transfected, and the mixture was incubated for 51-61 h at 37° C. Cells were quantified using CellTiter 96 Aqueous One Solution (Promega) or CellTiter-GLO Luminescence (Promega) following the manufacturer's recommendations.

Protein Detection

Cell lysates for western blotting were made in RIPA (Sigma) with protease inhibitor cocktail (Roche) and phosphatase inhibitor cocktails I and II (Santa Cruz Biotechnology). Western blots were probed with antibodies against p-MEK1/2 (S217/221), total MEK1/2, p-ERK1/2 (T202/Y204), total ERK1/2, PDGFRβ, and EGFR (all from Cell Signaling Technologies), B-RAF and N-RAS (Santa Cruz Biotechnology), pan-RAS (Thermo Scientific) and tubulin (Sigma). p-RTK arrays were performed according to the manufacturer's recommendations (Human Phospho-RTK Array Kit, R&D Systems). For PDGFRβ immunohistochemistry, paraffin-embedded formalin fixed tissue sections were subjected to antigen retrieval and incubated with a rabbit monoclonal anti-PDGFRβ antibody (Cell Signaling Technology) followed by labelled anti-rabbit polymer horseradish peroxidase (Envision System, Dako Cytomation). Immunocomplexes were visualized using the DAB (3,3′-diaminobenzidine) peroxidase method and nuclei haematoxylin-counterstained.

In Vitro Kinase Assay

Cells were harvested and protein lysates prepared in a NP40-based buffer before subjected to immunoprecipitation (IP). IP beads were then resuspended in ADBI buffer (with Mg/ATP cocktail) and incubated with an inactive, recombinant MEK1 or a truncated RAF-1 (positive control) (Millipore), and with DMSO or 1 μM PLX4032 for 30 min at 30° C. The beads were subsequently pelleted and the supernatant resuspended in sample buffer for western blotting to detect p-MEK and total MEK.

Activated RAS Pull-Down Assay

Melanoma lysates were incubated with glutathione agarose beads coupled to 80 μg GST-RAF-1-RBD (Thermo) for 1 h at 4° C. As controls, Pt48 R lysate was pre-incubated with either 0.1 mM GTPγS (positive control) or 1 mM GDP (negative control) in the presence of 10 mM EDTA (pH 8.0) at 30° C. for 15 min. Reactions were terminated by adding 60 mM MgCl₂. After washing with Wash Buffer (Thermo), proteins bound to beads were eluted by protein sample buffer. RAS or NRAS levels were detected by immunoblotting.

Quantitative Real-Time PCR for Relative RNA Levels

Total RNA was extracted using the RiboPure Kit (Ambion), and reverse transcription reactions were performed using the SuperScript First-Strand Synthesis System (Invitrogen). Real-time PCR analyses were performed using the iCycler iQ Real Time PCR Detection System (BioRad) (Table 4). To discriminate specific from nonspecific cDNA products, a melting curve was obtained at the end of each run. Data were normalized to tubulin and/or GAPDH levels in the samples in duplicates. Relative expression is calculated using the delta-Ct method using the following equations: ΔACt(Sample)=Ct(Target)−Ct(Reference); relative quantity=2^{-Δα}.

TABLE 4
Quantifying mRNA and gDNA copy numbers: genes, primers and real-time
PCR conditions.

| mRNA | Forward (SEQ ID NOs: 94-98) | Reverse (SEQ ID NOs: 99-103) |
|---|---|---|
| N-RAS | ACAGTGCCATGAGAGACCAA | TCGCTTAATCTGCTCCCTGT |
| B-RAF | ATGTTGAATGTGACAGCACC | CTCACACCACTGGGTAACAA |
| PDGFRβ | TTCCATGCCGAGTAACAGAC | CGTTGGTGATCATAGGGGAC |
| Tubulin | GACAGCTCTTCCACCCAGAG | TGAAGTCCTGTGCACTGGTC |
| GAPDH | CAATGACCCCTTCATTGACC | GACAAGCTTCCCGTTCTCAG |
| gDNA | Forward (SEQ ID NOs: 104-105) | Reverse (SEQ ID NOs: 106-107) |
| N-RAS | TTGGATTGTGTCCGTTGAGC | ACCCTGAGTCCCATCATCAC |
| Globin | AATTCACCCCACCAGTGCAG | CTTCCCGTTCTCAGCCTTGA |

A single step at 95° C. for 10 min preceded 40 cycles of amplification (95° C. for 30 s, 52° C. for 30 s, and 72° C. for 30 s). Subsequently, melting curve analysis was performed as follows: 95° C. for 10 s, 52° C. for 10 s, and 95° C. for 10 s.

Quantitative Real-Time PCR for Relative DNA Copy Numbers

gDNAs were extracted using the FlexiGene DNA Kit (Qiagen) (Human Genomic DNA-Female, Promega). NRAS relative copy number was determined by quantitative PCR (cycle conditions available upon request) using the MyiQ single colour Real-Time PCR Detection System (Bio-Rad). Total DNA content was estimated by assaying β-globin for each sample (Table 4), and 20 ng of gDNA was mixed with the SYBR Green QPCR Master Mix (Bio-Rad) and 2 pmol l⁻¹ of each primer.

Sequencing

gDNAs were isolated using the Flexi Gene DNA Kit (QIAGEN) or the QIAamp DNA FFPE Tissue Kit. B-RAF and RAS genes were amplified from genomic DNA by PCR. PCR products were purified using QIAquick PCR Purification Kit (QIAGEN) followed by bi-directional sequencing using BigDye v1.1 (Applied Biosystems) in combination with a 3730 DNA Analyzer (Applied Biosystems). PDGFRβ was amplified from cDNA by PCR and sequenced (primers listed in Table 1).

B-RAF Ultra-Deep Sequencing

Exon-based amplicons were generated using Platinum high-fidelity Taq polymerase, and libraries were prepared following the Illumina library generation protocol version 2.3. For each sample, one library was generated with 18 exons pooled at equal molarity and another library was generated for exon 13 only for validation purpose. Each library was indexed with an unique four base long barcode within the custom made Illumina adaptor. All 10 indexed samples were pooled and sequenced on one lane of Illumina GAllx flow-cell for single-end 76 base pairs. For error rate estimation, phiX174 genome was spiked in. Base-calling was performed by Illumina RTA version 1.8.70. Alignment was performed using the Novocraft Short Read Alignment Package version 2.06 (http://www.novocraft.com/index.html). First, all reads were aligned to the phiX174 reference genome downloaded from the NCBI. The mismatch rates at each position of the reads were calculated to estimate the error rate of the sequencer (set at 1.67% or five standard deviations, SD) based on the phiX genome data (mean error rate=0.57%, s.d.=0.22%). Then, the .qseq.txt files were converted into .fastq file using a custom script (available on request) and during this process, the first 5 bases (unique 4-base barcode and the T at the fifth position) were stripped off from the reads and concatenated to the read name. The .fastq file was parsed into 10 .fastq files for each barcode and only the reads with the first 5 bases perfectly matching any of the 10 barcodes were included. Each .fastq file was aligned to chromosome 7 fasta file, generated from the Human Genome reference sequence (hg18, March 2006, build 36.1) downloaded from the Broad Institute (ftp://ftp.broadinstitute.org/pub/gsa/gatk_resources.tgz) using the Novoalign program. Base calibration option was used, and the output format was set to SAM. Using SAMtools (http://samtools.sourceforge.net/), the .sam files of each lane were converted to .bam files and sorted, followed by removal of potential PCR duplicates using Picard (http://picard.sourceforge.net/). The true background rate was inferred from analysis of independent exon 13 amplicons. None of the 14 positions within exon 13 that had non-reference allele frequency (NAF)>1.67% in all-exon-samples were validated in the exon13-only samples and vice versa for the one position in the exon 13-only sample, inferring that the true background error rate could be higher at 4.81% (5s.d., mean error rate=2.72%, s.d.=0.4%). In total, 12 positions had NAF>4.81%, and none of them recurred at the same position. We note that the four sample gDNAs extracted from formalin-fixed paraffin-embedded (FFPE) blocks had 5-6 times more variants with NAF above background than the sample extracted from frozen tissue, and the 12 positions with NAF>4.81% were scattered only across the FFPE samples. The numbers of variants within and outside the kinase domain were not significantly different.

B-RAF Deep sequence from Whole Exome Sequence Analysis

Genomic libraries were generated following the Agilent SureSelect Human All Exon Kit Illumina Paired-End Sequencing Library Prep Version 1.0.1 protocol at the UCLA Genome Center. Agilent SureSelect All Exon ICGC version was used for capturing ˜50 megabase (Mb) exome. The Genome Analyzer IIx (GAIIx) was run using standard manufacturer's recommended protocols. Base-calling was done by Illumina RTA version 1.6.47. Two lanes of Illumina single end (SE) run were generated for each of Pt111-001 normal, baseline and DP2 samples, and one lane of Illumina paired end (PE) run was generated for each of Pt111-001 DP1, DP3 as well as Pt111-010 normal, baseline, DP1 and DP2 samples. Alignment was performed using the Novocraft Short Read Alignment Package version 2.06. Human Genome reference sequence (hg18, March 2006, build 36.1), downloaded from the UCSC genome database located at http://genome.ucsc.edu and mirrored locally, was indexed using novoindex program (−k 14 −s 3). Novoalign program was used to align each lane's qseq.txt file to the reference genome. Base calibration option and adaptor stripping option for paired-end run were used and the output format was set to SAM. Using SAMtools (http://samtools.sourceforge.net/), the .sam files of each lane were converted to .bam files, sorted and merged for each sample and potential PCR duplicates were removed using Picard (http://picard.sourceforge.net/). The .bam files were filtered for SNV calling and small INDEL calling to reduce the likelihood of using spuriously mis-mapped reads to call the variants. For the .bam file to call SNVs, the last 5 bases were trimmed and only the reads lacking indels were retained. For the .bam file to call small INDELs, only the reads containing one contiguous INDEL but not positioned at the beginning or the end of the read were retained. SOAP consensus-calling model implemented in SAMtools was used to call the variants, both SNVs and indels, and generate the .pileup files for each .bam file. Coding regions ±2 by of BRAF gene were extracted from the .pileup files and the reads were manually examined for rare variants (non reference alleles).

Microarray Data Generation and Analysis

Total RNAs were extracted using the RiboPure Kit (Ambion) from cells (DMSO or PLX4032, 1 M, 6 h). cDNAs were generated, fragmented, biotinylated, and hybridized to the GeneChip Human Gene 1.0 ST Arrays (Affymetrix). The arrays were washed and stained on a GeneChip Fluidics Station 450 (Affymetrix); scanning was carried out with the GeneChip Scanner 3000 7G; and image analysis with the Affymetrix GeneChip Command Console Scan Control. Expression data were normalized, background-corrected, and summarized using the RMA algorithm implemented in the Affymetrix Expression ConsoleTM version 1.1. Data were log-transformed (base 2) for parametric analysis. Clustering was performed with MeV 4.4, using unsupervised hierarchical clustering analysis on the basis of Pearson correlation and complete/average linkage clustering. Differentially expressed genes were identified using significance analysis of microarrays (SAM) with the R package ‘samr’ (R 2.9.0; FDR<0.05; fold change greater than 2). To identify and rank pathways enriched among differentially expressed genes, P-values (Fisher's exact test) were calculated for gene sets with at least 20% differentially expressed genes. Curated gene sets of canonical pathways in the Molecular Signatures Database (MSigDB) were used.

Copy Number Variation Analysis

Illumina HumanExon510S-DUO bead arrays (Illumina) were performed following the manufacturer's protocol. Scanned array data were imported into BeadStudio software (Illumina), where signal intensities for samples were normalized against those for reference genotypes. Log₂ ratios were calculated, and data smoothed using the median with window size of 10 and step size of five probes.

Cell Cycle and Apoptosis Analysis

All infected cells were replenished with PLX4032 24 h after infections (M229 R5 treated with AZD6244 to inhibit rebound p-ERK on PDGFRβ KD), fixed, permeabilized, and treated with RNase (Qiagen). Cells were stained with 50 mg ml⁻¹ propidium iodide (BD Pharmingen) and the distribution of cell cycle phases was determined by Cell Quest Pro and ModiFit software. For apoptosis, post-infection cells were stained with Annexin V-V450 (BD Pharmingen) and propidium iodide for 15 min at room temperature. Flow cytometry data were analysed by the FACS Express V2 software.

Image Acquisition and Data Processing

Statistical analyses were performed using InStat 3 Version 3.0b (GraphPad Software), and graphical representations using DeltaGraph or Prism (Red Rock Software). An Optronics camera system was used in conjunction with Image-Pro Plus software (MediaCybernetics) and Adobe Photoshop 7.0.

### References Cited in Example 1

1. Davies, H. et al. Nature 417, 949-954 (2002).

2. Flaherty, K. T. et al. N. Engl. J. Med. 363, 809-819 (2010).

3. Jänne, P. A., Gray, N. & Settleman, J. Nature Rev. Drug Discov. 8, 70 9-723 (2009).

4. Montagut, C. et al. Cancer Res. 68, 4853-4861 (2008).

5. Poulikakos, P. I., et al. Nature 464, 427-430 (2010).

6. Sondergaard, J. N. et al. J. Transl. Med. 8, 39-50 (2010).

7. Halaloban R. et al. Pigment Cell Melanoma Res. 23, 190-200 (2010).

8. Hatzivassiliou, G. et al. Nature 464, 431-435 (2010).

9. Heidorn, S. J. et al. Cell 140 209-221 (2010)

10. Bollag G. et al. Nature; 467, 596-599 (2010).

11. Tsai J. et al. Proc. Natl Acad. Sci. USA 105, 3041-3046 (2008).

12. Whittaker S. et al. Sci. Transl. Med. 2, 35ra41 (2010).

13. Pratilas, C. A. et al. Proc. Natl Acad. Sci. USA 106, 4519-4524 (2009).

14. Packer, L. M., et al. Pigment Cell Melanoma Res. 22, 785-798 (2009).

15. Wu, E et al. PLoS ONE 3, e3794 (2008).

16. Smith, G. et al. Br. J. Cancer 102, 693-703 (2010).

17. Emery, C. M. et al. Proc. Natl Acad. Sci. USA 106, 20411-20416 (2009).

18. Dumaz, N. et al. Cancer Res. 66, 9483-9491 (2008).

Supplementary Information is linked to the online version of the paper at www.nature.com/nature. A figure summarizing the main result of this paper is also included as SI. Gene expression and copy number data are deposited at Gene Expression Omnibus under accession numbers GSE24862 and GSE24890, respectively.

### Example 2

### Acquired Resistance to RAF Inhibitors is Mediated by Splicing Isoforms of BRAF(V600E) that Dimerize in a RAS Independent Manner

This example demonstrates a novel resistance mechanism. We find that a subset of cells resistant to PLX4032 (vemurafenib) express a 61 kd variant form of BRAF(V600E) that lacks exons 4-8, a region that encompasses the RAS-binding domain. p61 BRAF(V600E) exhibits enhanced dimerization as compared to full length BRAF(V600E) in cells with low levels of RAS activation. In cells in which p61 BRAF(V600E) is expressed endogenously or ectopically, ERK signaling is resistant to the RAF inhibitor. Moreover, a mutation that abolishes the dimerization of p61 BRAF(V600E) restores its sensitivity to PLX4032. Finally, we identified BRAF(V600E) splicing variants lacking the RAS-binding domain in the tumors of six of 19 patients with acquired resistance to PLX4032. These data support the model that inhibition of ERK signaling by RAF inhibitors is dependent on levels of RAS-GTP too low to support RAF dimerization and identifies a novel mechanism of acquired resistance in patients: expression of splicing isoforms of BRAF(V600E) that dimerize in a RAS-independent manner.

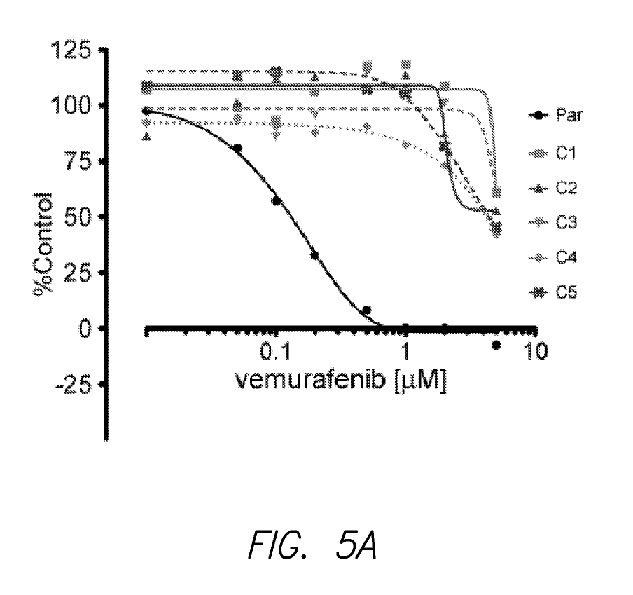

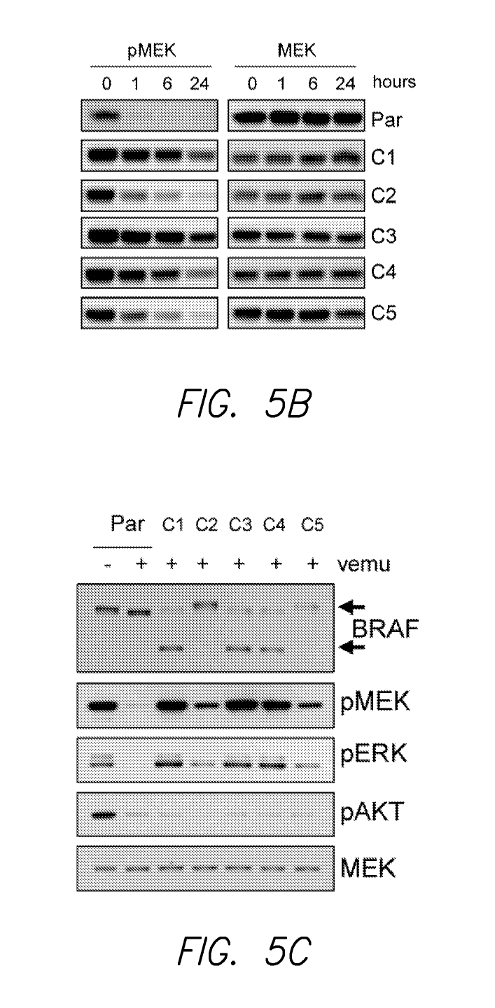

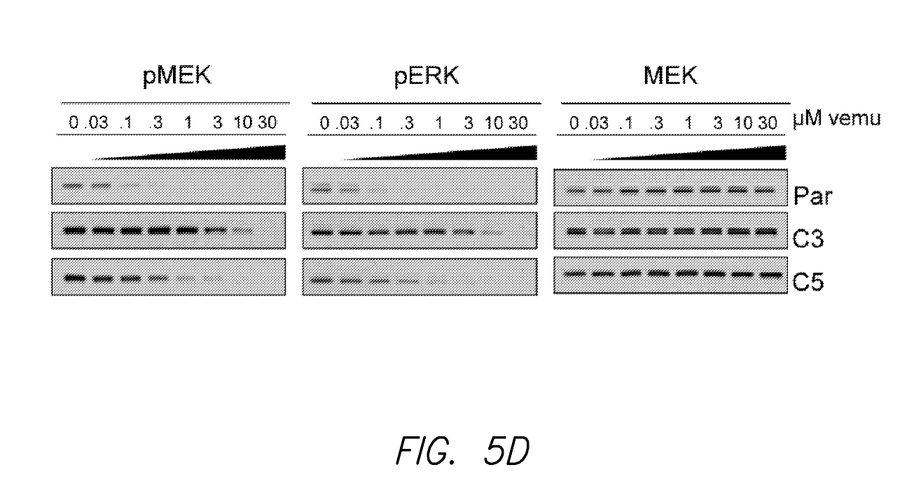

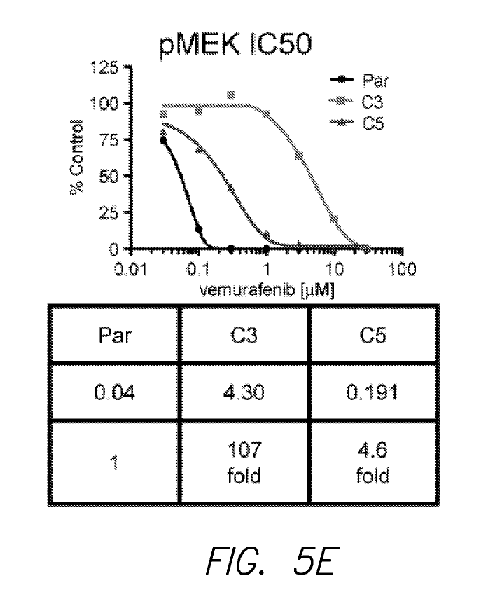

RAF inhibitors have remarkable clinical activity in mutant BRAF melanomas that is limited by acquisition of drug resistance⁸. In order to identify novel mechanisms of resistance, we generated cell lines resistant to PLX4032 by exposing the BRAF mutant (V600E) melanoma cell line SKMEL-239 to a high dose of drug (2 μM). At this concentration, PLX4032 effectively inhibits ERK signaling in SKMEL-239 and causes accumulation of cells in G1 and a significant induction of cell death (FIG. 5A-C). Five independent PLX4032-resistant cell populations were generated after approximately 2 months of continuous drug exposure (FIG. 5A). We chose this approach rather than one of gradual adaptation to increasing concentrations of drug since it more closely represents the clinical situation⁸.

Resistance of SKMEL-239 cells to PLX4032 was associated with decreased sensitivity of ERK signaling to the drug (FIGS. 5B, C). Analysis revealed the presence of two distinct classes of resistant clones. In the first, exemplified by the C3 clone, the IC50 for pMEK inhibition was more than 100-fold higher than that of the parental cell line (FIGS. 5D, E). Despite a similar degree of resistance to the anti-proliferative and pro-apoptotic effects of the drug, the second class of clones, exemplified by clone C5, demonstrated only a modest increase in pMEK IC50 (4.5-fold higher than the parental cell line). All five resistant clones retained sensitivity to the MEK inhibitor PD0325901¹³, albeit at slightly higher doses.

Analysis of both DNA and cDNA derived from the five resistant clones showed that all retained expression of BRAF(V600E). No mutations in BRAF at the gatekeeper site¹⁴, RAS mutation, upregulation of receptor tyrosine kinase activation or COT overexpression were detected. Analysis of BRAF protein expression showed that each of the resistant clones expressed a 90 kd band that co-migrated with the band observed in parental cells. In the C1, C3 and C4 clones, a new more rapidly migrating band was also identified, which ran at an approximate molecular weight of 61 kd (p61 BRAF(V600E), FIG. 5C). No band of this size was detected in parental SKMEL-239 cells or in a panel of 22 other melanoma cell lines.

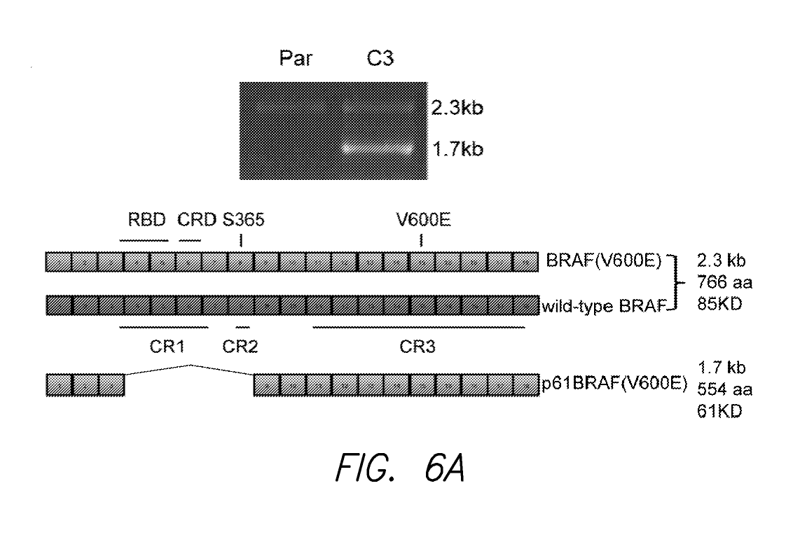

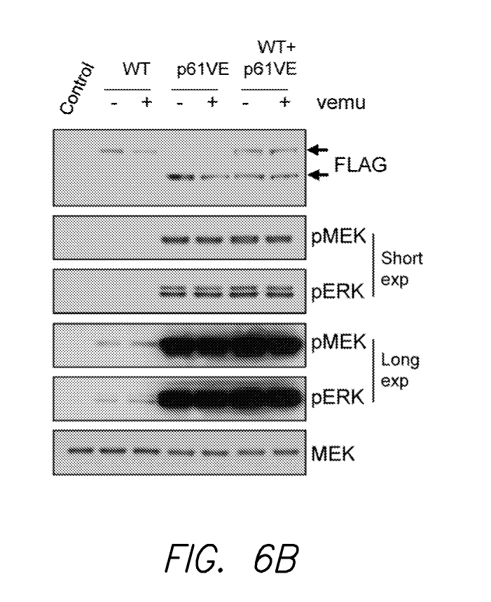

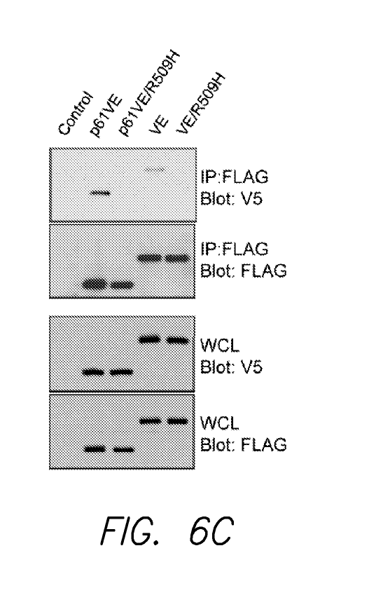

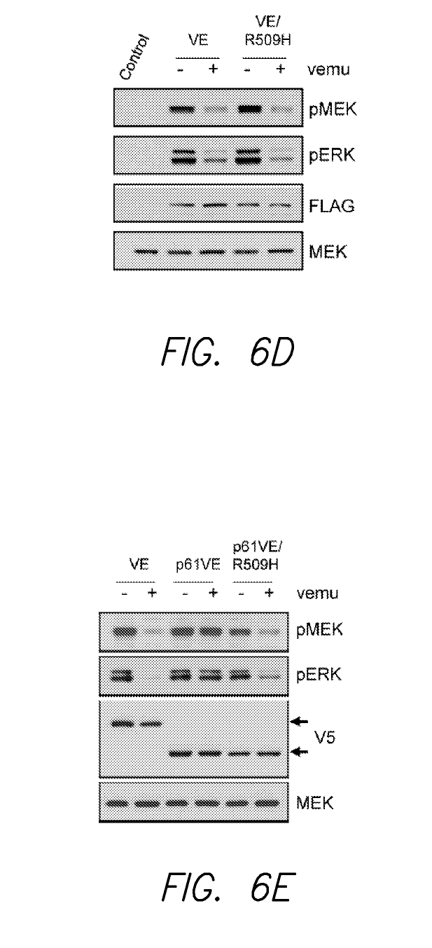

PCR analysis of cDNA derived from the parental and resistant cell lines revealed the expected single transcript of 2.3 kb, representing full-length BRAF in parental cells and two transcripts of 2.3 kb and 1.7 kb respectively in C3 cells. Sequence analysis of the 1.7 kb PCR product from C3 cells revealed that it was a BRAF transcript that contained the V600E mutation and an in-frame deletion of exons 4-8 (FIG. 6A). This 1.7 kb transcript is predicted to encode a protein of 554 amino acids and a molecular weight of 61 kd, consistent with the lower band detected by immunoblotting with the anti-BRAF antibody. Exons 4-8 encodes the majority of conserved regions 1 (CR1) and 2 (CR2) of BRAF, which include domains critical for RAF activation, most notably, the RAS-binding domain (RBD) and the cysteine-rich domain (CRD)³. Analogous deletions in the context of wild-type BRAF and CRAF have been generated experimentally and been shown to promote RAF dimerization, rendering RAS activity dispensable for this process^{1,4}. The 61 kd BRAF variant identified in C3 was also detected in clones C1 and C4 by real time PCR, with a primer that anneals specifically to the exon 3/9 junction. Inspection of the BRAF locus on chromosome 7q34 by array CGH data suggested no evidence of an intragenic somatic deletion within the BRAF gene.

The 1.7 kb transcript was cloned into an expression vector and expressed in 293H cells, alone or together with full-length wild-type BRAF. As shown in FIG. 6B, ERK signaling was resistant to PLX4032 in 293H cells in which p61 BRAF(V600E) was ectopically expressed. Furthermore, expression of p61 BRAF(V600E) in parental SKMEL-239 cells or in HT-29 (BRAF(V600E)) colorectal carcinoma cells resulted in failure of PLX4032 to effectively inhibit ERK signaling. To test whether ERK signaling in C3 cells was dependent on p61 BRAF(V600E), we used siRNAs directed against either the 3/9 splice junction or a region within the exon 4-8 deletion to selectively suppress the expression of p61 BRAF(V600E) or full length BRAF, respectively. In parental cells, ERK signaling was inhibited by knockdown of full-length BRAF(V600E). In contrast, in C3 cells, phosphorylation of MEK and cell growth were inhibited upon knockdown of p61 BRAF(V600E) but not full-length BRAF, ARAF or CRAF. Moreover, in C3 cells in which the expression of wild-type, full-length BRAF or CRAF was knocked down, ERK signaling remained resistant to PLX4032.

PLX4032 inhibits the kinase activity of RAF immunoprecipitated from cells, but activates intracellular RAF in BRAF wild-type cells⁴. This suggests that the conditions required for transactivation in vivo are not recapitulated in the in vitro assay. We tested whether p61 BRAF(V600E) is also sensitive to this inhibitor in vitro. Although the in vitro activity of p61 BRAF(V600E) was slightly higher than full-length BRAF(V600E), similar concentrations of PLX4032 cause their inhibition in vitro. These data indicate that resistance of p61 BRAF(V600E) to PLX4032 is not due to its inability to bind the inhibitor.

It has been shown that the N-terminus of RAF negatively regulates the C-terminal catalytic domain¹⁵ and that truncation of the N-terminus results in constitutive dimerization of the protein in the absence of activated RAS¹. We thus asked whether deletion of exons 4-8 promotes dimerization of p61 BRAF(V600E). 293H cells contain wild-type BRAF, but RAS-GTP levels are too low to support appreciable activation of ERK signaling by RAF inhibitors. To determine levels of dimerization, we co-expressed two constructs encoding the same protein (either p61 BRAF(V600E) or full-length BRAF(V600E)) but with different tags (Flag or V5). When expressed in 293H cells, dimerization of p61 BRAF(V600E) was significantly elevated compared to that of full-length BRAF(V600E) (FIG. 6C). The R509 residue (analogous to R401 in CRAF) is within the BRAF dimerization interface. Mutation of this residue to a histidine significantly diminishes dimerization of wild-type BRAF and results in loss of its catalytic activity in cells^{4,16}. However, full length BRAF(V600E/R509H) expressed in 293H cells retained its ability to fully activate ERK signaling and remained sensitive to PLX4032 (FIG. 6D). Moreover, BRAF(V600E/R509H) fully activates ERK signaling when expressed in either BRAF-null or ARAF/CRAF-null MEFs. These results confirm that BRAF(V600E) can signal as a monomer and support the idea that elevated RAS-GTP levels and RAF dimerization are necessary for the activation of wild-type RAF proteins but not the BRAF(V600E) mutant.

Our model implies that in tumors with BRAF(V600E), elevation of RAS-GTP or alterations that cause increased RAF dimerization in the absence of RAS activation would confer resistance to RAF inhibitors^{4,17}. To test whether resistance mediated by p61 BRAF(V600E) was the result of elevated dimer formation, we introduced the R509H dimerization-deficient mutation into p61 BRAF(V600E). In 293H cells expressing p61 BRAF(V600E), phosphorylation of ERK was elevated and was insensitive to PLX4032 (FIG. 6E). ERK activity was also elevated in cells expressing p61 BRAF(V600E/R509H), but to a slightly lesser degree. At the levels expressed, p61 BRAF(V600E/R509H) does not dimerize in these cells, confirming that the R509H mutation located within the dimerization interface disrupts the formation of p61 RAF(V600E) dimers (FIG. 6C). This monomeric p61 BRAF(V600E/R509H) was sensitive to RAF inhibitors as in cells ectopically expressing this mutant, ERK signaling was inhibited by PLX4032 (FIG. 6E). Thus, the R509H mutation both prevents the RAS-independent dimerization of p61 BRAF(V600E) and sensitizes it to the RAF inhibitor. These data confirm that deletion of exons 4-8 from BRAF(V600E) causes it to become insensitive to RAF inhibitors because it dimerizes in a RAS-independent manner.

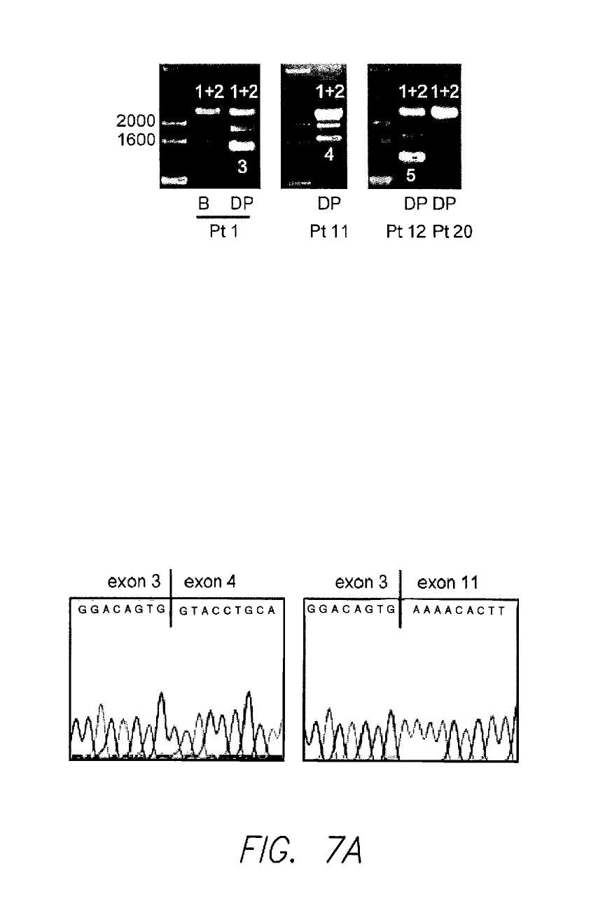

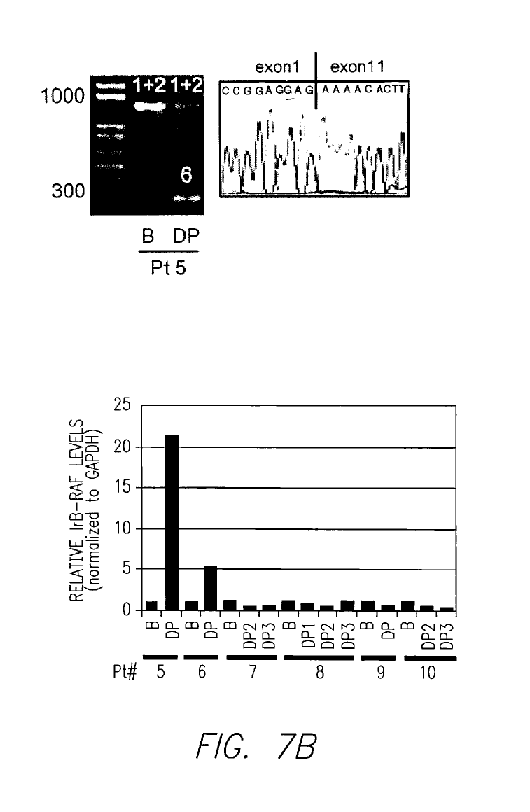

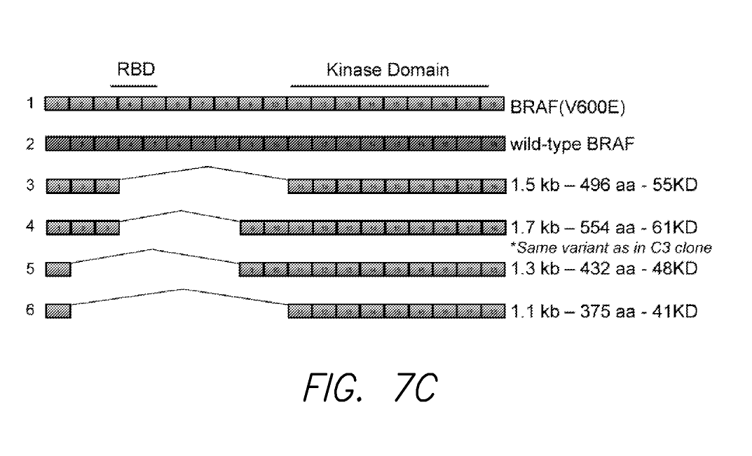

To determine whether BRAF variants can account for clinical resistance to RAF inhibitors, we analyzed tumors from nineteen melanoma patients with acquired resistance to PLX4032. We performed PCR analysis of cDNA from these tumors and the resulting products were sequenced. Pre-treatment samples showed a single band of the expected size (2.3 kb) which was sequenced and confirmed to include both BRAF(V600E) and wild-type BRAF transcripts (FIG. 7A). In six of the post-treatment progression samples, including three with matching pre-treatment samples, we identified two PCR products. In one of these patients with matching pre-treatment and post-treatment samples, PCR analysis of the sample collected at the time of disease progression revealed a shorter band encoding a BRAF(V600E) transcript lacking exons 4-10 (FIG. 7A-C, Patient I). In a second post-treatment tumor (patient II), the shorter transcript represented a BRAF(V600E) variant lacking exons 4-8, a transcript identical to the variant identified in the C1, C3 and C4 clones (FIGS. 7A, C). Additional post-treatment samples were found to express BRAF(V600E) variants that lacked exons 2-8 (patient III) or exons 2-10 (patient V, VI and 19) (FIGS. 7B, 7C). We detected NRAS mutations in 4 of the 19 progression samples (patient 2, 10, 16 and 17), with the mutant to WT NRAS ratio in patient 2 being low. In contrast, we did not detect MEK1 mutations in any of the 19 progression samples. Finally, two samples derived from patients with intrinsic resistance (patient IV shown) expressed only a single band encoding full-length BRAF as did twenty-seven additional melanomas resected from PLX4032-naive patients, 18 of which were V600E BRAF (FIG. 7A).

TABLE 5
Clinical Characteristics of the melanoma tumors. Data for patients I-IV is shown in FIG. 7.

| | | Study | | | Best | | | |
|---|---|---|---|---|---|---|---|---|
| Pt | | Site | stage | Age | Response | PFS | Bx Timing | Site of Biopsy |
| 1 | Del: 4-10 | Vanderbilt | M1c | 45F | −53% | 106 days | Baseline | Soft Tissue |
| | | | | | | | Progression | Soft Tissue |
| 2 | NRAS | Vanderbilt | M1a | 70F | −70% | 168 days | Baseline | Right Neck |
| | | | | | | | Progression | Muscle Mass-Thigh |
| 3 | | MGH | M1b | 73M | −72% | 464 days | Baseline | Groin LN |
| | | | | | | | Progression | Pelvic LN |
| 4 | | MGH | M1a | 41M | −20% | 304 days | Baseline | Soft Tissue |
| | | | | | | | Progression | Soft Tissue |
| 5 | Del: 2-10 | UCLA | M1c | 52M | −70% | 104 days | Baseline | SC R Axilla |
| | | | | | | | Progression | SC Trunk |
| 6 | Del: 2-10 | UCLA | M1c | 65M | −22% | 161 days | Baseline | SC Trunk |
| | | | | | | | Progression | SC Trunk |
| 7 | | UCLA | M1b | 66F | −53% | 137 days | Baseline | Cutaneous Neck |
| | | | | | | | Progression | Cutaneous L Neck |
| | | | | | | | | (DP1), L |
| | | | | | | | | Clavicular/Neck (DP2, |
| | | | | | | | | Same Site as PreTx |
| | | | | | | | | Bx), L Shoulder/Neck |
| | | | | | | | | (DP3) |
| 8 | | UCLA | IIIc | 47F | −31% | 238 days | Baseline | Cutaneous Leg |
| | | | | | | | Progression | Cutaneous - Left |
| | | | | | | | | Foot (DP1), Left |
| | | | | | | | | Leg(DP2), L Leg, |
| | | | | | | | | lateral (DP3) |
| 9 | | UCLA | M1c | 51M | −60% | 212 days | Baseline | SC - Scalp |
| | | | | | | | Progression | SC- Right Chest |
| 10 | NRAS | UCLA | M1c | 65F | −72% | 373 days | Baseline | SC - Trunk |
| | | | | | | | Progression | SC - Left |
| | | | | | | | | Flank/Buttock (DP1); |
| | | | | | | | | Soft tissue - left |
| | | | | | | | | breast (DP2/3-same |
| | | | | | | | | repeated site) |
| 11 | Del: 4-8 | Vanderbilt | M1c | 62M | −47% | 148 days | Progression | Lung |
| 12 | Del: 2-8 | Vanderbilt | M1a | 67M | −43% | 299 days | Progression | Groin mass |
| 13 | | MSKCC | M1c | 77M | −25% | 107 days | Progression | Soft tissue Mass- |
| | | | | | | | | scapula |
| 14 | | MSKCC | M1b | 54M | −74% | 149 days | Progression | Right Axillary Mass |
| 15 | | UCLA | M1c | 48M | −24% | 126 days | Progression | Cutaneous L Thigh |
| 16 | NRAS | UCLA | M1c | 65F | −37% | 279 days | Progression | SC - L groin |
| 17 | NRAS | UCLA | M1c | 30M | −100% | 118 days | Progression | SC - Trunk |
| 18 | | UCLA | M1c | 85M | −42% | 241 days | Progression | SC - R Shoulder |
| 19 | Del: 2-10 | UCLA | M1c | 47F | −31% | 168 days | Progression | Soft tissue - L Breast |
| 20 | | Vanderbilt | M1c | 51F | +8 | 79 days | Primary | Axillary LN |
| | | | | | | (till >20%) | Resistant | |
| 21 | | MSKCC | M1c | 23M | N/A | 50 | Primary | Soft Tissue Mass - |
| | | | | | | | Resistant | Thigh |

In tumors from patients that have been analyzed, resistance to PLX4032 is typically associated with inability of the drug to inhibit ERK signaling¹⁸. Our model suggests that this can be due to increased dimer formation in the cell⁴. This can happen in at least two likely mutually exclusive ways: increasing RAS-GTP levels and induction of RAS-independent dimerization. NRAS mutation has now been reported in resistant tumors⁹. Now, for the first time, we report a lesion that causes increased, RAS-independent dimerization in patient tumors. Other mechanisms of resistance to RAF inhibitors in model systems and in patients have also been reported recently and include activation of the receptor tyrosine kinases PDGFRβ and IGF1R^{9,11}. Another MEK kinase, COT, that can bypass the requirement of BRAF(V600E) for ERK signaling has also been shown to cause resistance as has mutation of MEK1^{10,12}.

p61 BRAF(V600E) is the first resistance mechanism identified that involves a structural change in BRAF. Notably, the alternative splicing forms identified in the cell lines and patients have all been confined to the mutant BRAF allele. This suggests that generation of the splice variants is likely due to a mutation or epigenetic change that affects BRAF splicing and not to a loss of global splicing fidelity¹⁹. The identification of BRAF variants lacking the RAS-binding domain in six of nineteen patients with acquired resistance suggests that this mechanism is clinically important and suggests novel treatment strategies. As resistance to PLX4032 resulting from expression of p61 BRAF(V600E) is attributable to attenuation of the ability of the drug to inhibit RAF activation, one would predict that such tumors would retain sensitivity to inhibitors of downstream effectors of RAF such as MEK, which was indeed the case. Therefore, MEK inhibitors if used in combination with PLX4032 may delay (or prevent) the onset of this mechanism of resistance or overcome resistance once established, with both hypotheses now being tested in ongoing clinical trials.

Methods Summary

PLX4032⁷ (vemurafenib) was obtained from Plexxikon Inc. PD0325901 was synthesized in the MSKCC Organic Synthesis Core Facility by O. Ouerfelli. Flag-tagged BRAF constructs have been described previously⁴. All other plasmids were created using standard cloning methods, with pcDNA3.1 (Invitrogen) as a vector. Mutations were introduced using the site-directed Mutagenesis Kit (Stratagene). The C1-5 PLX4032-resistant cells were generated by continuous exposure of parental SKMEL239 cells to 2 μM of drug until the emergence of resistant colonies. Single cell cloning was then performed prior to biological characterization.

For cDNA preparation, the Superscript III First-Strand Synthesis kit (Invitrogen) was used. Primers designed for the N- and C-termini of BRAF had the following sequences: N-terminus GGCTCTCGGTTATAAGATGGC (SEQ ID NO:108) and C-terminus: ACAGGAAACGCACCATATCC (SEQ ID NO: 109). Sanger sequencing of the products was performed by Genewiz. For qPCR analysis, cDNA synthesis was carried out with the High Capacity cDNA Reverse Transcription Kit (Applied Biosystems). qPCR was performed with the iQ SYBR Green RT-PCR Super Mix (BioRad) and the C1000 Thermal Cycler (BioRad). The comparative Ct method was employed to quantify transcripts and delta Ct was measured in triplicate. Primers for the total amount of BRAF: F-TCAATCATCCACAGAGACCTC (SEQ ID NO:110); R-GGATCCAGACAACTGTTCAAAC (SEQ ID NO:111); 3_{—}9 Junction: F-ACAAACAGAGGACAGTGGAC (SEQ ID NO:112); R-TTAGTTAGTGAGCCAGGTAATGA (SEQ ID NO:113).

Melanoma tumor specimens from patients treated with vemurafenib (PLX4032) on an IRB-approved protocol were flash frozen immediately after resection or biopsy. To determine tumor content, 5 μm sections from frozen patient tumor specimens were cut, stained with hematoxylin and eosin, and scored by a pathologist. If the specimen had >70% tumor content (excluding necrosis), the remainder of the frozen tumor was homogenized using a Bullet Blender (Next Advance, Inc.) with 0.9-2 mm stainless steel beads for 5 min at a speed setting of 10. RNA was then extracted from the tumor homogenate using the RNeasy Mini Kit (Invitrogen) and quantified.

Methods

Compounds. PLX4032 (vemurafenib) was obtained from Plexxikon Inc. PD0325901 was synthesized in the MSKCC Organic Synthesis Core Facility by O. Ouerfelli. Drugs were dissolved in DMSO and stored at −20° C.

Cell proliferation and cell cycle analysis. All melanoma cell lines were generated by A. Houghton (MSKCC) or obtained from ATCC. 293H cells were obtained from Invitrogen. Cells were maintained in DMEM (293H and MEFs), or RPMI (all other cell lines) supplemented with 2 mM glutamine, antibiotics and 10% fetal bovine serum. We confirmed by DNA fingerprinting²⁰ that all PLX4032-resistant, SKMEL-239 clones were derived from the same patient, thus excluding the possibility of contamination (Table 6). For proliferation assays, cells were plated in 6 well plates and 24 hours later were treated with varying concentrations of inhibitors as indicated. IC₅₀ values were calculated using Graph Pad Prism v.5. For cell cycle and apoptosis studies, cells were seeded in 6 well dishes the day prior to drug treatment. For analysis, both adherent and floating cells were harvested and stained with ethidium bromide as described previously²¹.

TABLE 6
Parental and PLX4032-resistant clones were confirmed to be derived from
the same patient using a mass spectrometry-based fingerprinting assay.

| | | | Bonferroni |
|---|---|---|---|
| Sample 1 | Sample 2 | p-Value | correction |
| SKMEL-239 | SKMEL-239 C1 | 8.05037E−15 | 6.85247E−11 |
| Parental | | | |
| SKMEL-239 | SKMEL-239 C2 | 8.05037E−15 | 6.85247E−11 |
| Parental | | | |
| SKMEL-239 | SKMEL-239 C3 | 8.05037E−15 | 6.85247E−11 |
| Parental | | | |
| SKMEL-239 | SKMEL-239 C4 | 8.05037E−15 | 6.85247E−11 |
| Parental | | | |
| SKMEL-239 | SKMEL-239 C5 | 8.05037E−15 | 2.97542E−11 |
| Parental | | | |

Western blotting and receptor tyrosine kinase (RTK) arrays. Western blot analysis was performed as previously described¹³. The following antibodies were used: p217/p221-MEK (pMEK), p202/p204-ERK (pERK), MEK, ERK, (Cell Signaling), V5 tag (Invitrogen), BRAF, cyclin Flag tag, β-actin (Sigma). For immunoprecipitations of tagged proteins: anti-Flag M2 affinity gel (Sigma). The Human Phospho-RTK array Kit (R&D Systems) was utilized to detect kinase activation within a panel of RTKs. Briefly, cells were plated in 10 cm dishes and harvested after 24 hours. Following lysis, 500 μg of lysate was applied to a membrane-anchored RTK array and incubated at 4° C. for 24 hours. Membranes were exposed to chemiluminescent reagents and images captured using the ImageQuant LAS 4000 instrument (GE HealthCare).

Plasmids/Trasfections. Flag-tagged BRAF constructs have been described previously⁴. All other plasmids were created using standard cloning methods, with pcDNA3.1 (Invitrogen) as a vector. Mutations were introduced using the site-directed Mutagenesis Kit (Stratagene). For transfection studies, cells were seeded at 35 mm or 100 mm plates and transfected the following day using Lipofectamine 2000 (Invitrogen). Cells were collected 24 hours later for subsequent analysis.

Immunoprecipitations and kinase assays. Cells were lysed in lysis buffer (50 mM Tris, pH7.5, 1% NP40, 150 mM NaCl, 10% glycerol, 1 mM EDTA) supplemented with protease and phosphatase inhibitor cocktail tablets (Roche). Immunoprecipitations were performed at 4° C. for 4 h, followed by three washes with lysis buffer and, in cases of subsequent kinase assay, one final wash with kinase buffer (25 mM Tris, pH 7.5, 10 mM MgCl₂). Kinase assays were conducted in the presence of 200 μM ATP at 30° C. for 20 min with inactive MEK(K97R) (Millipore) as a substrate. The kinase reaction was terminated by adding sample buffer and boiling. Kinase activity was determined by immunoblotting for pMEK.

siRNA knockdown. In order to selectively knock down p61 BRAF(V600E) or full-length BRAF, siRNA duplexes were designed to target the junction between exons 3-9 (JC-1 and JC-2) or sequences within exons 4-8 (ex[4-8]-1 and ex[4-8]-2. The sequences are the following: JC-1: GGACAGUGGACUUGAUUAGUU (SEQ ID NO:114), JC-2: AGGACAGUGGACUUGAUUAUU (SEQ ID NO:115), ex[4-8]-1: ACUGAUAUUUCCUGGCUUAUU (SEQ ID NO:116), ex[4-8]-2: CUGUCAAACAUGUGGUUAUUU (SEQ ID NO:117). To knock down ARAF and CRAF we used siRNA pools. All siRNA duplexes were from Dharmacon and transfections were carried out with Lipofectamine 2000 (Invitrogen) at a final siRNA concentration of 50nM, according to the manufacturer's instructions. 72 hours later, cells were either counted to estimate cell growth, or subjected to immunoblot analysis.

### References Cited in Example 2

1 Weber, C. K., Slupsky, J. R., Kalmes, H. A. & Rapp, U. R. Cancer Res 61, 3595-3598 (2001).

2 Rushworth, L. K., Hindley, A. D., O'Neill, E. & Kolch, W. Mol Cell Biol 26, 2262-2272 (2006).

3 Wellbrock, C., Karasarides, M. & Marais, R. Nat Rev Mol Cell Biol 5, 875-885 (2004).

4 Poulikakos, P. I., et al. Nature 464, 427-430 (2010).

5 Heidorn, S. J. et al. Cell 140, 209-221, doi:S0092-8674 (2010).

6 Hatzivassiliou, G. et al. Nature 464, 431-435 (2010).

7 Joseph, E. W. et al. Proc Natl Acad Sci USA 107, 14903-14908 (2010).

8 Flaherty, K. T. et al. N Engl J Med 363, 809-819 (2010).

9 Nazarian, R. et al. Nature 468, 973-977 (2010).

10 Johannessen, C. M. et al. Nature 468, 968-972 (2010).

11 Villanueva, J. et al. Cancer Cell 18, 683-695 (2010).

12 Wagle, N. et al. J Clin Oncol (2011).

13 Solit, D. B. et al. Nature 439, 358-362 (2006).

14 Whittaker, S. et al. Sci Transl Med 2, 35ra41 (2010).

15 Cutler, R. E., Jr., et al. Proc Natl Acad Sci USA 95, 9214-9219 (1998).

16 Rajakulendran, T., et al. Nature 461, 542-545 (2009).

17 Poulikakos, P. I. & Rosen, N. Cancer Cell 19, 11-15 (2011).

18 McArthur, G. et al. J Clin Oncol 29, suppl; abstr 8502 (2011).

19 Luco, R. F., et al. Cell 144, 16-26 (2011).

20 Janakiraman, M. et al. Cancer Res 70, 5901-5911 (2010).

21 Nusse, M., Beisker, W., Hoffmann, C. & Tarnok, Cytometry 11, 813-821 (1990).

### Example 3

### Melanoma Exome Sequencing Identifies V600EB-RAF Amplification-Mediated Acquired Vemurafenib Resistance

This example demonstrates whole exome sequencing of melanoma tissues from patients treated with vemurafenib or GSK2118436 to uncover ^{V600E}B-RAF copy number gain as a bona fide mechanism of acquired B-RAFi resistance. In 20 patients studied, ^{V600E}B-RAF copy number gain was detected in four patients (20%) and was mutually exclusive with detection of N-RAS mutations ^{V600E}B-RAF truncation, or upregulation of receptor tyrosine kinases (RTKs), which are established mechanisms of acquired B-RAFi resistance^{8,10,11.} In isogenic drug-sensitive and -resistance cell line pairs, ^{V600E}B-RAF over-expression conferred vemurafenib resistance, whereas its knockdown sensitized the resistant sub-lines to B-RAFi. In ^{V600E}B-RAF amplification-driven B-RAFi resistance, in contrast to mutant N-RAS-driven resistance, ERK reactivation is saturable, with higher doses of vemurafenib down-regulating pERK and re-sensitizing melanoma cells to B-RAFi. These two mechanisms of ERK reactivation were differentially sensitive to the MEK1/2 inhibitor AZD6244/selumetinib or its combination with the B-RAFi vemurafenib. Finally, unlike mutant N-RAS-mediated ^{V600 E}B-RAF bypass, which is sensitive to C-RAF knockdown, ^{V600E}B-RAF amplification-mediated resistance functions largely independently of C-RAF. Thus, distinct clinical strategies may be required to overcome ERK reactivation underlying acquired resistance to B-RAFi in melanoma.

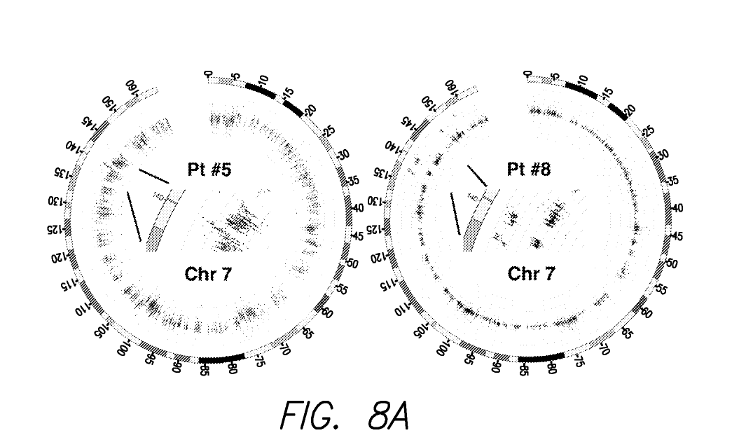

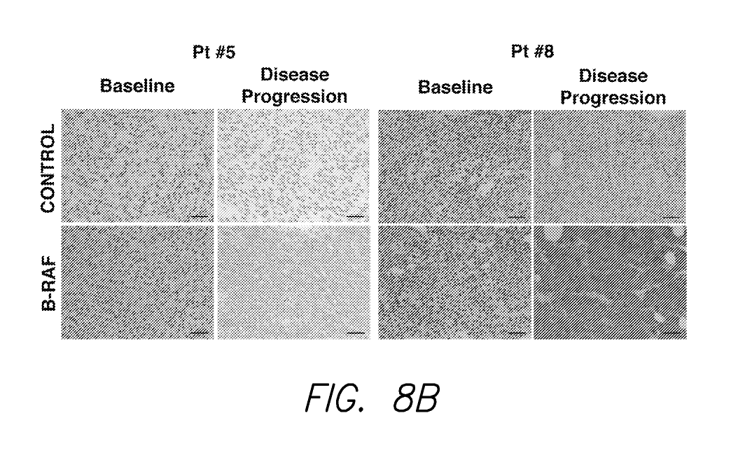

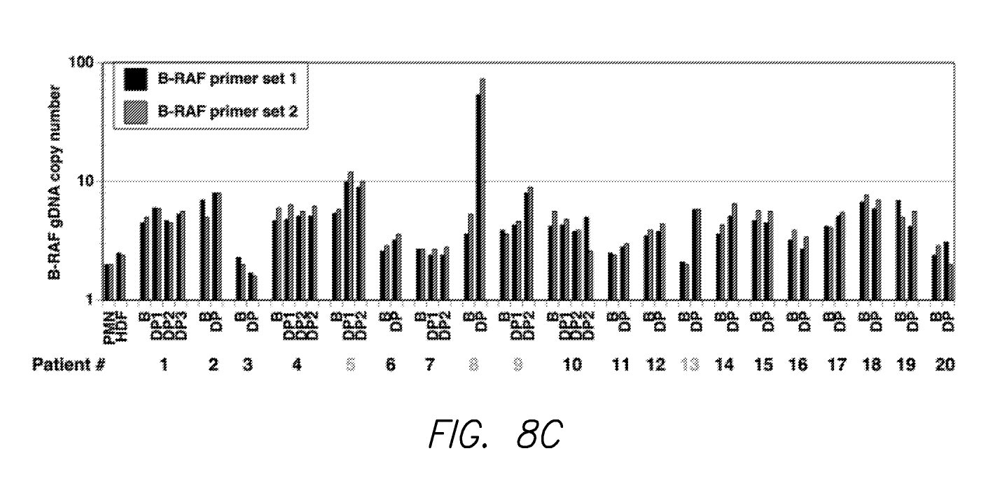

We assembled twenty sets of patient-matched baseline (prior to B-RAFi therapy) and disease progression (DP) (i.e., acquired B-RAFi resistance) melanoma tissues and analyzed them to identify the reported mechanisms of acquired B-RAFi resistance in melanoma. These reported mechanisms include N-RAS¹⁰ and MEK1¹² mutations, alternative-spliced ^{V600 E}B-RAF variants¹¹, and over-expression of RTKs (PDGFRβ^{7,10}, IGF1-R⁸) and COT⁹ (Tables 5 and 6). For DP samples negative for these mechanisms and where there is sufficient frozen and patient-matched normal tissues (from patients #4, 5, 8, 14, 16, 17 & 18), we subjected triads of genomic DNAs (gDNAs) from normal, baseline, and DP tissues to whole exome sequencing. In two available data sets, we analyzed for somatic DP-specific non-synonymous single nucleotide variants (nsSNVs) and small insertion-deletion (indels), which were exceedingly few in number or absent, respectively, using our bioinformatic workflow (Tables 9 and 10). We also analyzed for DP-specific copy number variations (CNVs) from the exome sequence data (Table 9). This identified ^{V600 E}B-RAF copy number gains in these two patients' DP tissues (2.2 and 12.8 fold in patients #5 and 8, respectively) relative to their respective baseline tissues (FIG. 8A; Table 7). Gain in ^{V600 E}B-RAF copy number was reflected in corresponding increased gene expression in integrated RNA and protein level analysis (FIG. 8B).

^{V600E}B-RAF amplification was validated by gDNA Q-PCR, producing highly consistent fold increases in DP-specific ^{V600E}B-RAF copy number gain (relative to baseline) (2.0 and 14 fold increase in patient #5 and 8 respectively) (FIG. 8C). We then expanded the analysis of ^{V600 E}B-RAF amplification to all twenty paired melanoma tissues and detected ^{V600 E}B-RAF copy number gains in DP samples from two additional patients (2.3 and 3 fold for DP2 of patient #9 & DP of patient #13, respectively) (FIG. 8C; Tables 7 and 8). We note that these copy number fold increases are likely underestimates of the true changes due to tumor heterogeneity, as most disease progressive tumors occur from stable residual tumors as a result of partial responses seen in the vast majority of patients treated with B-RAF inhibitors. An increase in the mutant B-RAF to WT B-RAF ratio was also noted in all four cases of DP harboring B-RAF copy number gain when compared to their respective baseline tissues, consistent with selection for ^{V600E}B-RAF(vs. the WT B-RAF allele) copy number gain during acquisition of B-RAFi resistance. ^{V600E}B-RAF amplification was mutually exclusive with N-RAS mutations (no MEK1 exon 3 mutation detected), RTK over-expression (no COT over-expression detected), as well as a novel mechanism involving ^{V600E}B-RAF alternative splicing¹¹ (Table 7).

TABLE 7
Clinical characteristics and acquired resistance mechanisms in patients with
matched baseline and disease progression (DP) melanomas tissues.

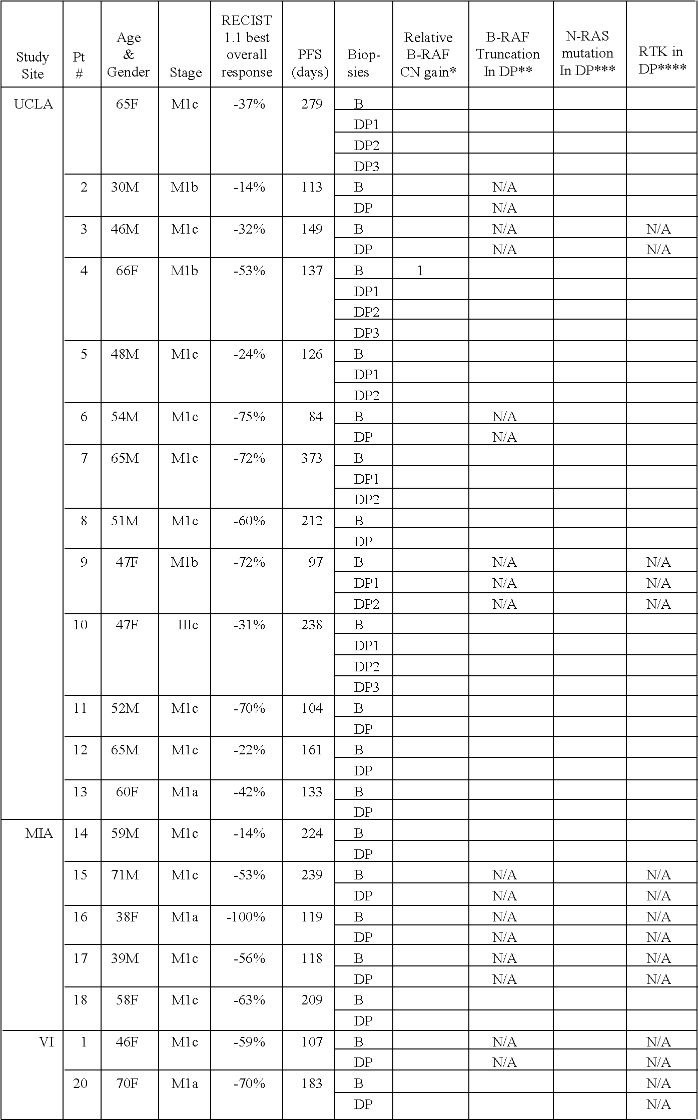

UCLA, University of California, Los Angeles; MIA, Melanoma Institute, Australia; VI, Vanderbilt-Ingram Cancer Center
Vemurafenib/PLX4032 treated patients, black; dabrafenib/GSK2118436 treated patients, purple Patient numbers 1, 4, 5, 7, 8, 10, 11, 12 and 20 correspond to patient numbers 16, 7, 15, 10, 9, 8, 5, 6, and 2, respectively, in Example 2 above.
Patient numbers 1, 2, 3, 4, 5, 6 and 19 correspond to patients 55, 48, 92, 111-001, 111-010, 104-004, and 56 in Example 1 above.
*See Fig 1c.
**No B-RAF secondary mutations were detected.
***H-RAS and K-RAS are WT in all DP samples.
****IHC data presented in Nazarian et al, Nature (2010) for Pt #2 and #6.
*****Only Exon 3 of MEK1 was sequenced. No MEK1 exon 3 mutation was detected in any DP sample.
Grey boxes represent negative findings in baseline tissues for each mechanism of acquired resistance identified. Dark boxes represent positive findings.

TABLE 8
Biopsy sites of patients studied.

| Study | | | |
|---|---|---|---|
| Site | Pt # | Biopsies | Anatomic Bx Sites |
| UCLA | 1 | B | Lymph node- femoral |
| | | DP1 | Lymph node- inguinal |
| | | DP2 | Small bowel |
| | | DP3 | SC and cutaneous- L groin |
| | 2 | B | SC- shoulder |
| | | DP | Heart |
| | 3 | B | Lymph node- R axillary |
| | | DP | Soft tissue- abdomen |
| | 4 | B | SC- L base of neck |
| | | DP1 | SC- L neck |
| | | DP2 | SC- L base of neck |
| | | DP3 | SC- L shoulder |
| | 5 | B | Lymph node- L inguinal |
| | | DP1 | Cutaneous- L ant thigh, |
| | | | superior |
| | | DP2 | Cutaneous- L ant thigh, |
| | | | inferior |
| | 6 | B | Lung |
| | | DP | Pelvic |
| | 7 | B | SC- L lower flank/buttock |
| | | DP1 | SC- L lower flank/buttock |
| | | DP2 | Soft tissue- L breast |
| | 8 | B | SC- scalp |
| | | DP | SC- R chest |
| | 9 | B | SC- abdomen |
| | | DP1 | SC- R chest |
| | | DP2 | Cutaneous- L shoulder |
| | 10 | B | Cutaneous- L leg |
| | | DP1 | Cutaneous- L foot |
| | | DP2 | Cutaneous- L leg, medial |
| | | DP3 | Cutaneous- L leg, lateral |
| | 11 | B | SC- R axillary |
| | | DP | SC- back |
| | 12 | B | SC- abdomen |
| | | DP | SC- R flank |
| | 13 | B | Soft tissue- pelvis |
| | | DP | Soft tissue- pelvis |
| MIA | 14 | B | SC- L chest |
| | | DP | SC- abdomen |
| | 15 | B | SC- Upper chest |
| | | DP | SC- abdomen |
| | 16 | B | Lymph node- R inguinal |
| | | DP | Brain |
| | 17 | B | Lymph node- R neck |
| | | DP | SC- R neck |
| | 18 | B | SC- L groin |
| | | DP | SC- L flank |
| VI | 19 | B | Lymph node- inguinal |
| | Pt56 | DP | Soft tissue- pelvis |
| | 20 | B | SC- R neck |
| | SL | DP | SC- R leg |

TABLE 9
Exome sequencing data characteristics.
Pt #8

| | Normal | Baseline | DP |
|---|---|---|---|
| Library | 50 + 50 PE, | 50 + 50 PE, | 50 + 50 PE, |
| | 100 + 100 PE | 100 + 100 PE | 100 + 100 PE |
| Total read count | 198,535,632 | 270,137,370 | 256,439,396 |
| Capture specificity | 43.2% | 44.1% | 42.3% |
| % of targeted base | 89.5% | 90.3% | 90.6% |
| covered at >=10x | | | |
| Average Coverage | 107.6 x | 132.6 x | 123.3 x |

| Type of somatic alterations | DP-specific # |
|---|---|
| Non-synonymous or nsSNVs | 4 |
| INDELs | 0 |
| CNVs | 871 (468: Amplified, 403: Deleted) |

Pt #5

| | Normal | Baseline | DP |
|---|---|---|---|
| Library | 76 SE | 76 + 76 PE | 76 + 76 PE |
| Total read count | 62,448,536 | 137,656,936 | 147,415,956 |
| Capture specificity | 75.2% | 78.1% | 74.7% |
| % of targeted base | 88% | 92% | 93% |
| covered at >=10x | | | |
| Average Coverage | 52.7 x | 88.8 x | 114.3 x |

| Type of somatic alterations | DP-specific # |
|---|---|
| Non-synonymous or nsSNVs | 1 |
| INDELs | 0 |
| CNVs | 734 (424: Amplified, 310: Deleted) |

TABLE 10
DP-specific somatic nsSNVs.

| | | | | | | | | | | | |
|---|---|---|---|---|---|---|---|---|---|---|---|
| | | | | P value | P value | | | | | | |
| | | | | (DP vs. | (DP vs. | | | AA | AA | PhyloP | |
| Chr. | Position | Re [symbol] | Var | normal) | base-line) | Accession ID | Gene | change | position | Score | Polyphen |

Pt #8

| | | | | | | | | | | | |
|---|---|---|---|---|---|---|---|---|---|---|---|
| 4 | 8609063 | C | T | 1.25E−004 | 4.28E−005 | NM_001014447 | CPZ | HIS/ | 380/653, | 5.362 | probably |
| | | | | | | NM_001014448 | | TYR | 243/516, | | damaging |
| | | | | | | NM_003652 | | | 369/642 | | |
| 4 | 101108952 | A | T | 3.23E−013 | 5.26E−017 | NM_145244 | DDIT4L | PHE/ | 155/194 | 2.674 | benign |
| | | | | | | | | TYR | | | |
| 4 | 109745350 | G | C | 1.53E−013 | 8.87E−017 | NM_032518, | COL25A1 | LEU/ | 609/643, | −0.041 | benign |
| | | | | | | NM_198721 | | VAL | 609/655 | | |
| 4 | 110791146 | C | T | 1.32E−022 | 1.62E−034 | NM_198506 | LRIT3 | PRO/ | 369/635 | 2.898 | possibly |
| | | | | | | | | LEU | | | damaging |
| 8 | 95839582 | G | C | 8.18E−023 | 5.33E−032 | NM_017864 | INTS8 | ALA/ | 133/996 | 0.907 | benign |
| | | | | | | | | PRO | | | |
| 8 | 124792307 | T | C | 6.02E−012 | 1.90E−020 | NM_144963 | FAM91A1 | VAL/ | 211/839 | 3.5 | benign |
| | | | | | | | | ALA | | | |
| 8 | 134125756 | C | T | 2.01E−009 | 6.69E−018 | NM_003235 | TG | ARG/ | 2555/2769 | 1.964 | probably |
| | | | | | | | | CYS | | | damaging |
| 10 | 11894129 | C | T | 1.59E−005 | 3.89E−006 | NM_153256 | C10orf47 | SER/ | 18/436 | 1.599 | probably |
| | | | | | | | | PHE | | | damaging |
| 10 | 13225081 | C | T | 2.07E−025 | 2.07E−025 | NM_018518, | MCM10 | PRO/ | 360/875, | 3.903 | possibly |
| | | | | | | NM_182751 | | LEU | 361/876 | | damaging |
| 10 | 17641343 | G | A | 1.22E−014 | 2.08E−015 | NM_014241 | PTPLA | SER/ | 184/289 | 6.163 | possibly |
| | | | | | | | | PHE | | | damaging |
| 10 | 19856502 | G | A | 4.96E−025 | 4.15E−025 | XM_295865 | C10orf112 | TRP/ | 1560/1818 | 4.889 | |
| | | | | | | | | stop | | | |
| 10 | 45878069 | G | A | 2.21E−007 | 1.95E−008 | NM_000698 | ALOX5 | GLU/ | 97/675 | 5.36 | probably |
| | | | | | | | | LYS | | | damaging |
| 10 | 79769683 | G | A | 2.58E−017 | 1.05E−014 | NM_007055 | POLR3A | SER/ | 570/1391 | 5.708 | benign |
| | | | | | | | | LEU | | | |
| 10 | 91520377 | C | T | 4.06E−016 | 3.43E−015 | NM_016195 | KIF20B | SER/ | 1552/1781 | 2.821 | possibly |
| | | | | | | | | PHE | | | damaging |
| 10 | 106982927 | G | A | 8.71E−040 | 1.25E−051 | NM_014978 | SORCS3 | GLU/ | 930/1223 | 5.526 | possibly |
| | | | | | | | | LYS | | | damaging |
| 10 | 131665425 | G | T | 0.0225472 | 0.0336512 | NM_001005463 | EBF3 | PRO/ | 331/552 | 5.984 | probably |
| | | | | | | | | HIS | | | damaging |
| 13 | 113825980 | C | T | 7.00E−005 | 2.29E−007 | NM_003891 | PROZ | ALA/ | 255/401 | −0.203 | benign |
| | | | | | | | | VAL | | | |
| 14 | 70512882 | C | A | 0.009946 | 0.0068087 | NM_001130417, | SLC8A3 | VAL/ | 227/299, | 6.222 | probably |
| | | | | | | NM_033262, | | LEU | 854/926, | | damaging |
| | | | | | | NM_058240, | | | 853/925, | | |
| | | | | | | NM_182932, | | | 850/922, | | |
| | | | | | | NM_182936, | | | 213/285, | | |
| | | | | | | NM_183002 | | | 856/928 | | |
| 15 | 45393436 | C | T | 1.64E−007 | 5.54E−007 | NM_014080 | DUOX2 | ARG/ | 963/1549 | 2.902 | possibly |
| | | | | | | | | GLN | | | damaging |
| 19 | 45296786 | C | T | 0.0047006 | 0.0276535 | NM_001130852, | CBLC | ALA/ | 352/429, | 2.095 | Benign |
| | | | | | | NM_012116 | | VAL | 398/475 | | |

Pt #5

| | | | | | | | | | | | |
|---|---|---|---|---|---|---|---|---|---|---|---|
| 1 | 184556124 | C | A | 1.14E−11 | 1.07E−19 | NM_003292 | TPR | ASP/ | 2171/2364 | 4.96 | possibly |
| | | | | | | | | TYR | | | damaging |
| X | 100418099 | T | A | 1.86E−4 | 3.55E−07 | NM_024885 | TAF7L | GLU/ | 341/463 | −6.19 | neutral |
| | | | | | | | | ASP | | | |

[symbol]

indicates data missing or illegible when filed

TABLE 11
Primer and shRNA sequences.

| qRT-PCR | Forward (SEQ ID NOs: 118-124) | Reverse (SEQ ID NOs: 125-131) |
|---|---|---|
| PDGFRb | TTCCATGCCGAGTAACAGAC | CGTTGGTGATCATAGGGGAC |
| IGF1R | CCGCAGACACCTACAACATC | CAATGTGAAAGGCCGAAGGT |
| COT | CCCCTGGAAGCTGACTTACA | CTGGGATCAGTTTACACGCC |
| B-RAF | ATGTTGAATGTGACAGCACC | CTCACACCACTGGGTAACAA |
| trB-RAF | TGCCATTCCGGAGGAGAAAAC | AGGCTTGTAACTGCTGAGGTG |
| Tubulin | GACAGCTCTTCCACCCAGAG | TGAAGTCCTGTGCACTGGTC |
| GAPDH | CAATGACCCCTTCATTGACC | GACAAGCTTCCCGTTCTCAG |
| gDNA copy | Forward (SEQ ID NOs: 132-135) | Reverse (SEQ ID NOs: 136-138) |
| B-RAF set1 | ACCTCAGCAGTTACAAGCCT | CACTGGGAACCAGGAGCTAA |
| B-RAF set2 | GATATTGCACGACAGACTGCA | AGCATCCTTATGTTCCTGGACA |
| Globin | AATTCACCCCACCAGTGCAG | CTTCCCGTTCTCAGCCTTGA |
| shRNA primer | | |
| sequences | Sense (SEQ ID NOs: 139-148) | Antisense (SEQ ID NOs: 149-158) |
| shSCRAMBLED | TGGAATCTCATTCGATGCATACTT | TCGAGAAAAAAGGAATCTCATTCG |
| | CAAGAGAGTATGCATCGAATGAG | ATGCATACTCTCTTGAAGTATGCATC |
| | ATTCCTTTTTTC | GAATGAGATTCCA |
| ShC-RAF1 | TGACAGAGAGATTCAAGCTATTT | TCGAGAAAAAAACAGAGAGATTCA |
| | CAAGAGAATAGCTTGAATCTCTCT | AGCTATTCTCTTGAAATAGCTTGAAT |
| | GTTTTTTTC | CTCTCTGTCA |
| ShC-RAF3 | TGCAAAGAACATCATCCATAGTT | TCGAGAAAAAACAAAGAACATCATC |
| | CAAGAGACTATGGATGATGTTCT | CATAGTCTCTTGAACTATGGATGAT |
| | TTGTTTTTTC | GTTCTTTGCA |
| ShB-RAF1 | TGACAGAGACCTCAAGAGTAATT | TCGAGAAAAAAACAGAGACCTCAAG |
| | CAAGAGATTACTCTTGAGGTCTC | AGTAATCTCTTGAATTACTCTTGAGG |
| | TGTTTTTTTC | TCTCTGTCA |
| ShB-RAF3 | TGCAACAACAGGGACCAGATATT | TCGAGAAAAAACAACAACAGGGACC |
| | CAAGAGATATCTGGTCCCTGTTG | AGATATCTCTTGAATATCTGGTCCCT |
| | TTGTTTTTTC | GTTGTTGCA |

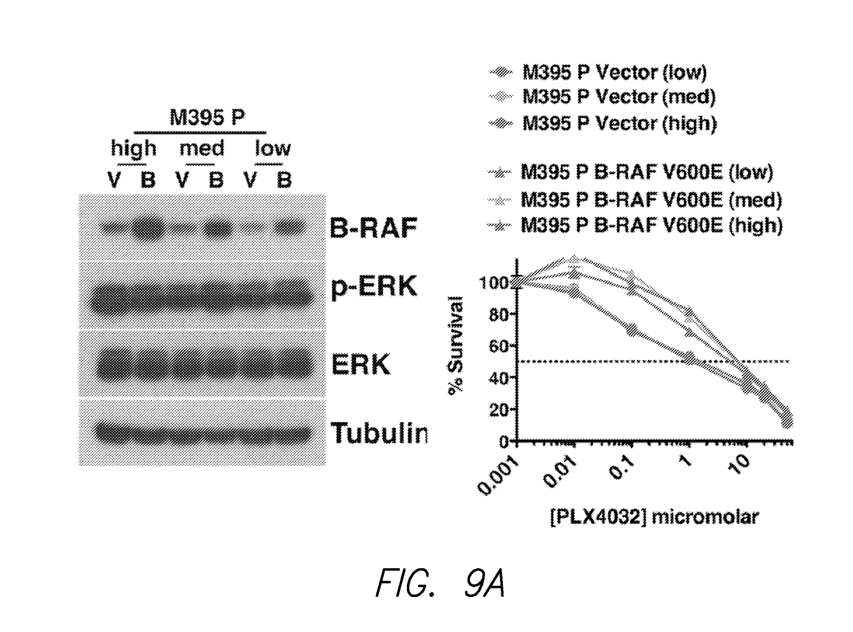

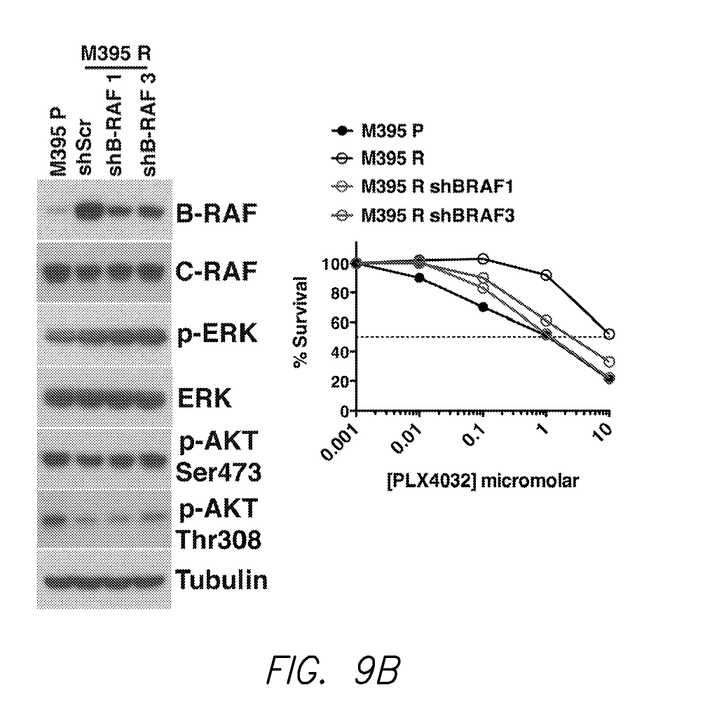

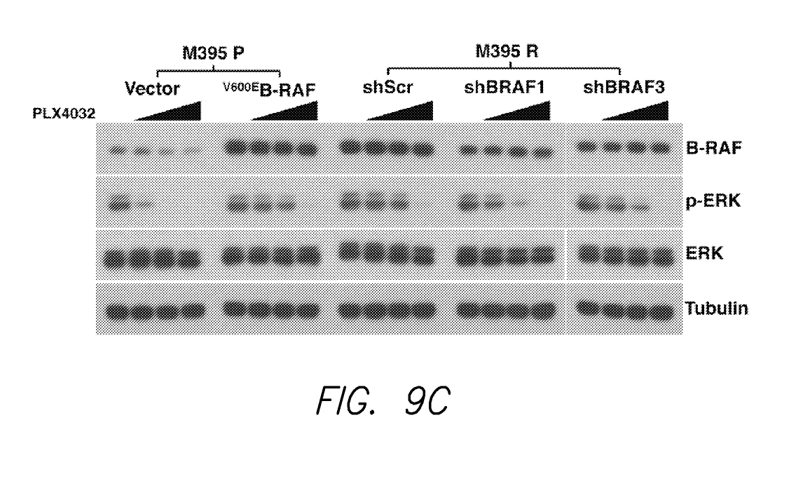

We have derived and analyzed vemurafenib/PLX4032-resistant (R) sub-lines derived by continuous vemurafenib exposure from seven human melanoma-derived ^{V600E}BRAF-positive parental (P) cell lines sensitive to vemurafenib-mediated growth inhibition. Four resistant sub-lines, including M229 R5 and M238 R1^{7,10}, over-expressed PDGFRβ compared to their parental counterpart. One sub-line (M249 R4¹⁰) gained a mutation in N-RAS, and another (M397 R) an alternatively spliced variant of ^{V600E}B-RAF resulting in in-frame fusion of exons 1 and 11. As in our tissue analysis, these mechanisms were identified in a mutually exclusive manner. Another vemurafenib-resistant sub-line, M395 R, was derived from a ^{V600E}B-RAF-homozygous parental line, M395 P. Compared to M395 P, M395 R harbors increased copy numbers of ^{V600E}B-RAF gDNA and cDNA, consistent with a dramatic ^{V600E}B-RAF protein over-expression. M395 R displays growth highly resistant to vemurafenib treatment, and titration of M395 R with vemurafenib (1 h) after a 24 h of drug withdrawal revealed pERK levels to be highly resistant to acute ^{V600E}B-RAF inhibition. This pattern of MAPK reactivation was similar to that seen in a mutant N-RAS-driven, vemurafenib-resistant sub-line, M249 R4, and contrasted with that in the RTK-driven vemurafenib-resistant sub-line, M229 R5^{7,10}. Expectedly, the levels of p-AKT are unchanged (see FIG. 9B below) comparing M395 P vs. M395 R, consistent with a lack of RTK over-expression leading to MAPK-redundant, PI3K-AKT signaling⁷. Accordingly, M395 R does not over-express either PDGFRβ or IGF-1R, in contrast to M229 R5, which has been shown to over-express the RTK PDGFRβ^{7,8}. Additionally, M395 R is WT for N-, H- and K-RAS and MEK1, harbors no secondary mutations in ^{V600E}B-RAF or an alternatively spliced variant of ^{V600E}B-RAF which results in a N-terminally truncated ^{V600E}B-RAF protein.

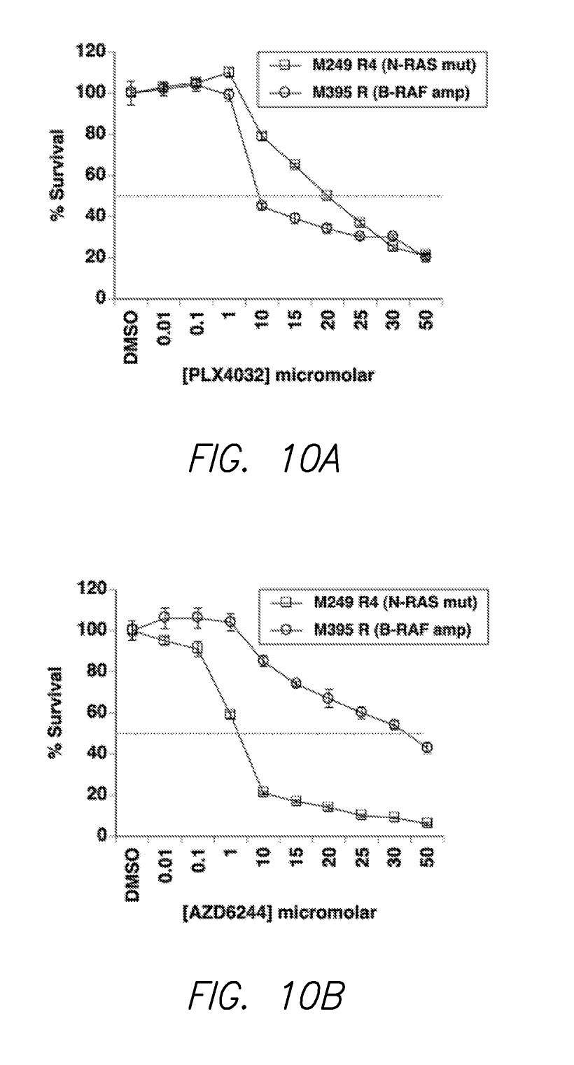

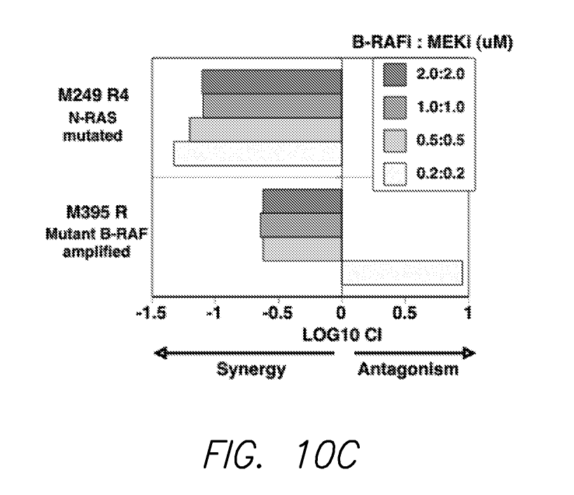

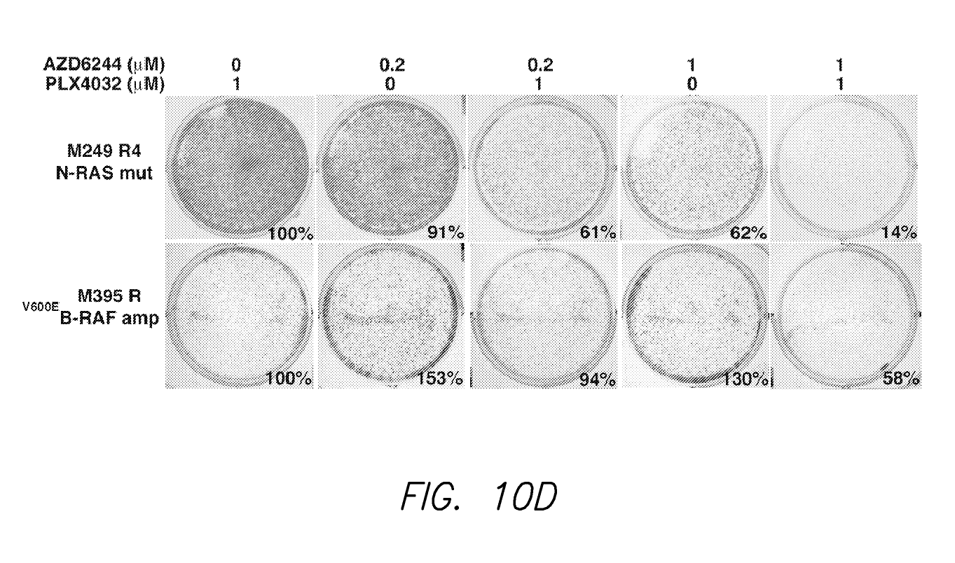

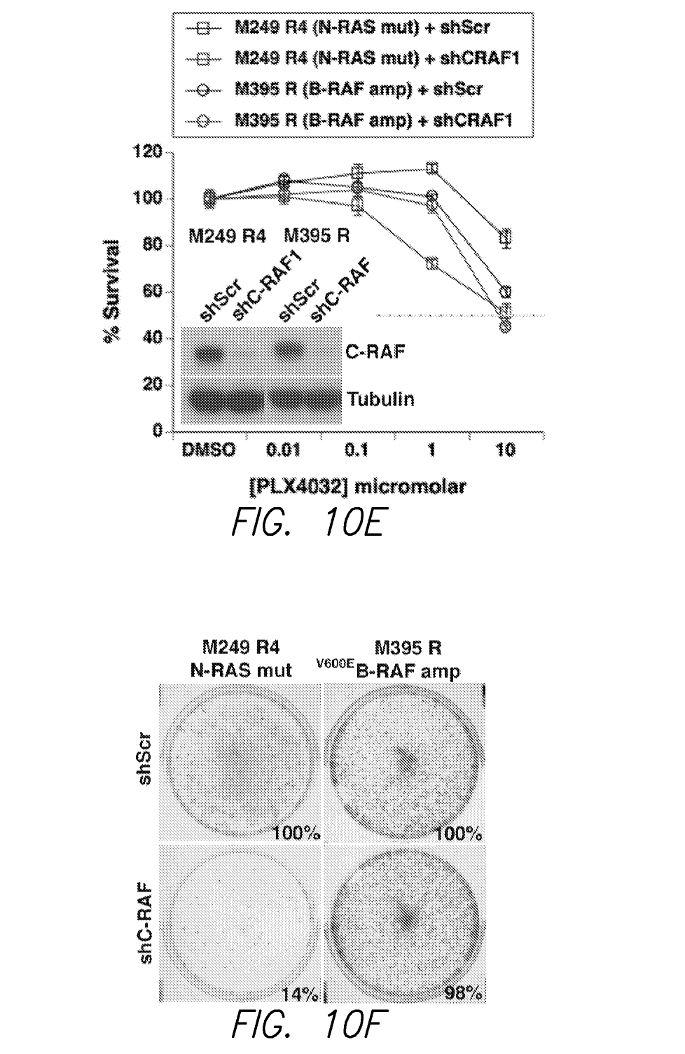

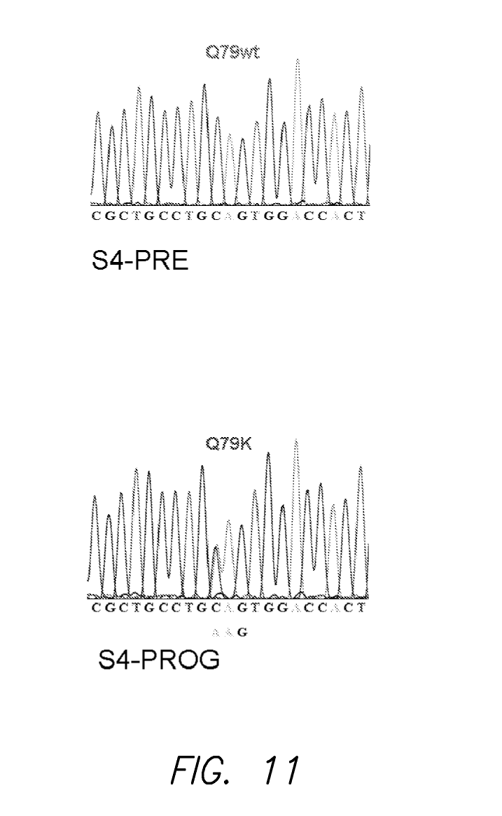

^{V600E}B-RAF over-expression in M395 P conferred vemurafenib resistance (FIG. 9A), but this resistance was highly saturable by micromolar concentrations of vemurafenib. Conversely, ^{V600E}B-RAF knockdown in M395 R confers vemurafenib sensitivity (FIG. 9B). Consistently, ^{V600E}B-RAF over-expression in M395 P (at levels titrated to be comparable to M395 R) and its knockdown in M395 R resulted in p-ERK resistance and sensitivity, respectively, to acute vemurafenib treatment after a 24 h drug withdrawal (FIG. 9C). We predicted that, regardless of the cellular genetic context, MAPK reactivation due to drug target (i.e., ^{V600E}B-RAF) over-expression would be saturable by higher doses of vemurafenib, in contrast to mutant N-RAS-mediated MAPK reactivation where ^{V600E}B-RAF may be bypassed by the alternative use of C-RAF¹³. Indeed, dosing of vemurafenib from 1 to 50 M revealed a significant difference in drug sensitivity of M249 R4 (^{Q61K}N-RAS) vs. M395 R (amplified ^{V600E}B-RAF) (FIG. 10A) (where the latter was highly sensitive to vemurafenib at this drug concentration range), suggesting a potential therapeutic opportunity. To rule out these results were not due to a difference in genetic backgrounds, we artificially rendered the ^{V600E}B-RAF melanoma cell line, M229, vemurafenib-resistant by either ^{Q61K}N-RAS or ^{V600 E}B-RAF viral transduction. Consistently, high dose vemurafenib treatment was more effective at overcoming drug resistance in ^{V600E}B-RAF-transduced M229 than in the same cell line transduced with ^{Q61K}N-RAS. Since both N-RAS mutation and ^{V600E}B-RAF amplification-driven acquired resistance mechanisms would be anticipated to result in MEK reactivation, we tested the allosteric MEKi, AZD6244/selumetinib, on the ^{Q61K}N-RAS-driven M249 R4 and the ^{V600E}B-RAF amplification-driven M395 R sub-lines. MEKi treatment resulted in decreased proliferation in both cases, but the activity was noted at lower concentrations for the ^{Q61K}N-RAS-driven resistance mechanism (FIG. 10B). This differential pattern was reproducible by exposing AZD6244/selumetinib to ^{V600E}B-RAF melanoma cell lines M229 and M238 transduced with high levels of ^{V600E}B-RAF vs. a short-term culture, Pt55 R¹⁰, with ^{Q61K}N-RAS-driven acquired B-RAFi resistance. We also tested the combination of B-RAFi with MEKi, which is currently in clinical testing¹⁴, in three-day survival assays. A calculation of combination index (CI) values using equal ratios of vemurafenib and selumetinib was performed. The results were consistent with a highly synergistic effect of these two agents combined in overcoming both mutant N-RAS-driven (M249 R4) and ^{V600E}B-RAF amplification-driven B-RAFi resistance (M395 R) (FIG. 10C), although the combination tended to be more potent against mutant N-RAS-driven acquired resistance to vemurafenib. This B-RAFi and MEKi combinatorial synergy was further corroborated in longer-term clonogenic assays (FIG. 10D).

We also predicted that MAPK reactivation due to ^{V600E}B-RAF over-expression would be C-RAF-independent, in contrast to mutant N-RAS-mediated MAPK reactivation where ^{V600E}B-RAF may be bypassed by the alternative use of C-RAF. Indeed, C-RAF knockdown by shRNA sensitized the mutant N-RAS sub-line, M249 R4, but not the ^{V600E}B-RAF amplified sub-line, M395 R, to vemurafenib in three-day survival assays (FIG. 10E). C-RAF knockdown restored vemurafenib sensitivity to M249 R4 (^{Q61K}N-RAS/^{V600E}B-RAF) even more strikingly in a longer-term clonogenic assays which afforded fresh drug replacement every two days (FIG. 10F). An independent C-RAF shRNA also restored vemurafenib sensitivity to M249 R4. Notably, B-RAFi and MEKi synergy and C-RAF-dependence in mutant N-RAS-driven acquired B-RAFi resistance was confirmed in a short-term culture derived from a tumor with clinical acquired vemurafenib resistance.

Identification of ^{V600E}B-RAF amplification as a mechanism of acquired resistance in B-RAFi treated patients provides evidence for alterations in the drug target causing clinical relapse. Based on these studies, therapeutic stratification of MAPK reactivation underlying B-RAFi resistance into drug-saturable or C-RAF-dependent pathways may be translatable into the design of next-generation clinical trials aimed at preventing or overcoming B-RAFi resistance. These findings also provide pre-clinical rationale for dose escalation studies in selected patients with B-RAFi-resistant ^{V600E/K}B-RAF metastatic melanomas, particularly given the wide range of effective dosing and the fact that the maximum tolerated dose of GSK2118436 has not been determined. The combination of current B-RAF inhibitors (or next-generation RAF inhibitors that enhance B-RAF potency or feature pan-RAF inhibition) with MEK1/2 inhibitors may potentially broadly block MAPK reactivation.

Method Summary

Cell culture, infections and drug treatments. Cells were maintained in DMEM with 10 or 20% fetal bovine serum and glutamine. shRNAs for B-RAF and C-RAF were sub-cloned into the lentiviral vector pLL3.7; pBabe B-RAF (V600E) was purchase (plasmid 17544, Addgene); viral supernatants generated by co-transfection with three packaging plasm ids into HEK293T cells; and infections carried out with protamine sulfate. Stocks and dilutions of PLX4032 (Plexxikon, Berkeley, Calif.) and AZD6244 (commercially available) were made in DMSO. Cells were quantified using CellTiter-GLO Luminescence (Promega) or crystal violet staining followed by NIH Image J quantification.

Whole exome sequencing and exomeCNV¹⁵ analysis. Agilent SureSelect Human All Exon 50 mb (regular or XT) was used for exome capture and Illumina GAII and HiSeq2000 were used for sequencing following manufacturer's manual. The reads were aligned to the reference human genome (hg18 or b37) using Novoalign from Novocraft (http://www.novocraft.com) and processed with SAMtools¹⁶, Picard (http://picard.sourceforge.net/) and GATK (Genome Analysis Tool Kit)¹⁷ to have both SNVs and small indels called. SeattleSeqAnnotation was used for annotating the somatic variants and ExomeCNV¹⁵ was used for calling copy number variations.

Protein detection. Western blots were probed with antibodies against p-ERK1/2 (T202/Y204), ERK1/2, C-RAF, AKT (Ser473), AKT (Thr308), AKT (Cell Signalig Technologies), N-RAS, B-RAF (Santa Cruz Biotechnology), and tubulin (Sigma). For B-RAF immunohistochemistry, paraffin-embedded formalin-fixed tissue sections were antigen-retrieved, incubated with the primary antibody followed by HRP-conjugated secondary antibody (Envision System, DakoCytomation). Immunocomplexes were visualized using the DAB (3,3′-diaminobenzidine) peroxidase method and nuclei hematoxylin-counterstained.

Genomic DNA and RNA quantifications. For real-time quantitative PCR, total RNA was extracted and cDNA quantified by the iCycler iQ Real Time PCR Detection System (BioRad). Data were normalized to TUBULIN and GAPDH levels. Relative expression is calculated using the delta-Ct method. gDNAs were extracted using the FlexiGene DNA Kit (Qiagen) (Human Genomic DNA-Female, Promega). B-RAF relative copy number was determined by quantitative PCR (cycle conditions available upon request) using the MyiQ single color Real-Time PCR Detection System (Bio-Rad). Total DNA content was estimated by assaying -globin for each sample, and 20 ng of gDNA was mixed with the SYBR Green QPCR Master Mix (Bio-Rad) and 2 pmol/L of each primer. All primer sequences are provided in Table 11.

Methods

Whole exome sequencing. For each sample, 3 ug of high molecular weight genomic DNA was used as the starting material to generate the sequencing library. Exome captures were performed using Agilent SureSelect Human All Exon 50 mb and Agilent SureSelect Human All Exon 50 mb XT for PT #5 and Pt #8, respectively, per manufacturers' recommendation, to create a mean 200 bp insert library. For Pt #5, sequencing was performed on Illumina GenomeAnalyzerii (GAIi) as 76+76 bp paired-end run. The normal sample was run on 1 flowcell lane and the tumor samples were run on 2 flowcell lanes each. For Pt #8, sequencing was performed on Illumina HiSeq2000 as 50+50 bp paired-end run and 100+100bp paired-end run. The three samples (normal, baseline and DP) were initially mixed with 9 other samples and run across 5 flowcell lanes for the 50+50 bp run. For the 100+100 bp run, they were mixed with 3 other samples to be run across 5 flowcell lanes with barcoding of each individual genomic sample library.

For Pt #5, approximately 62 million, 137 million, 147 million reads were generated for normal tissue (skin), baseline melanoma and DP melanoma, respectively, with 75.2%, 78.1%, and 74.7% of the reads mapping to capture targets. Based on an analysis of reads that uniquely aligned to the reference genome and for which the potential PCR duplicates were removed, an average coverage of 52×, 88×, and 114× was achieved with 87%, 92% and 93% of the targeted bases being covered at 10× or greater read depth for normal, baseline and DP, respectively.

For Pt #8, approximately 198 million, 270 million, 256 million reads were generated for normal tissue (skin), baseline melanoma and DP melanoma, respectively with 43.2%, 44.1% and 42.3% of the reads mapping to capture targets. Based on an analysis of reads that uniquely aligned to the reference genome and for which the potential PCR duplicates were removed, an average read depth of 107×, 132× and 123× was achieved with 89%, 90% and 90% of the targeted bases being covered at 10× or greater for normal, baseline and DP, respectively.

Sequencing Data Analysis. For Pt #8 where the samples were indexed and pooled before the sequencing, Novobarcode from Novocraft was used to de-multiplex the data. The sequence reads were aligned to the human reference genome using Novoalign V2.07.13 from Novocraft (http://www.novocraft.com). For Pt #5, hg18 downloaded from UCSC genome database was used and for Pt #8, b37 downloaded from GATK (Genome analysis toolkit) resources website was used for the reference genome. SAMtools v.0.1.16¹⁶ was used to sort and merge the data and Picard (http://picard.sourceforge.net/) was used to mark PCR duplicates. To correct the misalignments due to the presence of indels, local realignment was performed using RealignerTargetCreator and IndelRealigner of GATK¹⁷. Indel calls in dbSNP132 were used as known indel input. Then, GATK CountCovariates and TableRecalibration were used to recalibrate the originally reported quality score by using the position of the nucleotide within the read and the preceding and current nucleotide information. Finally, to call the single nucleotide variants (SNVs), the GATK UnifiedGenotyper was used to the realigned and re-calibrated bam file while GATK IndelGenotyperV2 was used to call small insertion/deletions (Indels). To generate a list of somatic variants for DP tumor, the difference in allele distribution was calculated using one-sided Fisher's exact test using normal sample or the baseline sample. Variants with p-value<0.05 were included in the “somatic variant list”. Low coverage (<10×) SNVs and SNVs with more than one variant allele in normal tissue and baseline melanoma were filtered out during the process. These somatic variants were further annotated with SeattleSeqSNPannotation (http://gvs.gs.washington.edu/SeattleSeqAnnotation/). For DP-specific, non-synonymous SNVs that result in missense mutations , we assessed the level of amino acid conservation using PhyloP score (provided in UCSC genome database) where a score >2 implies high conservation and the nature of amino substitution using Polyphen-2 analysis¹⁸.

CNV analysis was performed using an R package, ExomeCNV¹⁵. ExomeCNV uses the ratio of read depth between two samples at each capture interval. Here, the read depth data between baseline and DP melanomas were compared. Briefly, the read depth information was extracted through the PILEUP file generated from the BAM file after removing PCR duplicates using SAMtools. The average read depth at each capture interval was calculatedand the classify.eCNV module of ExomeCNV was run with the default parameters to calculate the copy number estimate for each interval. Subsequently, another R package commonly used to segment the copy number intervals, DNAcopy¹⁹, was called through ExomeCNV multi.CNV.analyze module with default parameters to do segmentation and sequential merging. The genomic regions with copy number 1 were called deletion and any regions with copy number >2 were called amplification. Circos²⁰ was used to visualize the CNV data.

### References Cited in Example 3

1. Davies, H. et al. Nature 417, 949-54 (2002).

2. Bollag, G. et al. Nature 467, 596-9 (2010).

3. Chapman, P.B. et al. N Engl J Med 364, 2507-16 (2011).

4. Flaherty, K. T. et al. N Engl J Med 363, 809-19 (2010).

5. Kefford, R. et al. J Clin Oncol 28, suppl; abstr 8503 (2010).

6. Ribas, A. et al. BRIM-2: Journal of Clinical Oncology 29, suppl; abstr 8509 (2011).

7. Shi, H., Kong, X., Ribas, A. & Lo, R. S. Cancer Research 71, 5067-74 (2011).

8. Villanueva, J. et al. Cancer Cell 18, 683-95 (2010).

9. Johannessen, C. M. et al. Nature 468, 968-72 (2010).

10. Nazarian, R. et al. Nature 468, 973-7 (2010).

11. Example 2.

12. Wagle, N. et al. J Clin Oncol 29, 3085-96 (2011).

13. Dumaz, N. et al. Cancer Res 66, 9483-91 (2006).

14. Infante, J. R. et al. Journal of Clinical Oncology 29, suppl; abstr CRA8503 (2011).

15. Sathirapongsasuti, J. F. et al. Bioinformatics (2011).

16. Li, H. et al. Bioinformatics 25, 2078-9 (2009).

17. McKenna, A. et al. Genome Res 20, 1297-303 (2010).

18. Adzhubei, I. A. et al. Nat Methods 7, 248-9 (2010).

19. Olshen, A. B., Venkatraman, E. S., Lucito, R. & Wigler, M. Biostatistics 5, 557-72 (2004).

20. Krzywinski, M. et al. Genome Res 19, 1639-45 (2009).

### Example 4

### Acquired Resistance via AKT1 Mutation

This example demonstrates an additional mechanism of B-RAF inhibitor acquired resistance that develops with disease progression. Methods as described for Example 1 above were used to analyze melanoma cells obtained from a brain tumor biopsy to reveal a mutation in the serine-threonine protein kinase AKT1, namely Q79K. This novel mutation results in P13K-independent activation of AKT1. As indicated in FIG. 11 and Table 12 below, this mutation is found in the biopsy at disease progression but not in a melanoma tissue sampled before B-RAF inhibitor treatment. Thus, patients whose samples exhibit the same or similarly activating mutation in AKT1 are candidates for alternative therapy.

TABLE 12

| sample ID | Progression stage | AKT1 |
|---|---|---|
| S4 | PRE | wt |
| | PROG | Q79K |

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of to predicting development of acquired resistance to therapeutic effects of B-RAF inhibitor therapy in a patient suffering from cancer, the method comprising:
(a) obtaining a biological sample from the patient, wherein the biological sample is selected from blood, tumor biopsy, spinal fluid, and needle aspirates;
(b) testing the biological sample from (a) for a measure of B-RAF inhibitor resistance, wherein the measure of B-RAF inhibitor resistance is selected from:
(1) an alternative splice variant that lacks exons 2-10 or exhibits gene amplification of ^{V600E}B-RAF;
(2) hyperactivity or elevated levels of PDGFR-beta relative to a control sample; and
(3) an activating mutation of N-RAS or AKT1.

2. The method of claim 1, wherein the B-RAF inhibitor is vemurafenib.

3. The method of claim 1, wherein the measure of B-RAF inhibitor resistance is an alternative splice variant that lacks exons 2-10 or exhibits gene amplification of ^{V600E}B-RAF.

4. The method of claim 3, wherein the measure of B-RAF inhibitor resistance is hyperactivity or elevated levels of PDGFR-beta relative to a control sample.

5. The method of claim 2, wherein the measure of B-RAF inhibitor resistance is an activating mutation of N-RAS or AKT1.

6. The method of claim 1, which is performed prior to B-RAF inhibitor therapy.

7. The method of claim 1, which is performed after initiation of B-RAF inhibitor therapy.

8. The method of claim 1, wherein the testing for an alternative splice variant of B-RAF comprises amplification of B-RAF and detection of a transcript of approximately 1.7 kb or detection of a nucleic acid sequence for a junction between exons 1 and 11 that is CCGGAGGAG/AAAACACTT (SEQ ID NO: 162).

9. The method of claim 1, wherein the testing for hyperactivity or elevated levels of PDGFR-beta comprises assaying for PDGFR-beta mRNA, protein or phospho-protein, and wherein the assaying comprises detection of increased levels of PDGFR-beta relative to a control sample or increased levels phospho-tyrosine on PDGFR-beta relative to a control sample.

10. The method of claim 1, wherein the testing for-act a measure of B-RAF inhibitor resistance comprises assaying for an activating N-RAS mutation.

11. The method of claim 10, wherein the activating N-RAS mutation includes missense mutations at codon 12, 13 and 61.

12. The method of claim 10, wherein the activating N-RAS mutation is Q61K or Q61R.

13. The method of claim 1, wherein the testing for an indicator of N-RAS mutation comprises assaying for elevated levels of N-RAS gDNA, mRNA or protein copy number.

14. The method of claim 1, wherein the patient has a B-RAF-mutant cancer.

15. The method of claim 1, wherein the patient has a B-RAF-mutant melanoma.

16. A method of treating a patient having melanoma, the method comprising administering to the patient a MEK inhibitor, optionally in conjunction with vemurafenib therapy, or an inhibitor of the MAPK pathway (RAF, MEK, ERK) in conjunction with an inhibitor of the RTK-PI3K-AKT-mTOR pathway.

17-20. (canceled)

21. The method of claim 1, wherein the testing comprises polymerase chain reaction (PCR), mass spectrometry, or DNA sequencing.

22. The method of claim 1, which is performed after suspension of B-RAF inhibitor therapy.

23. The method of claim 1, wherein the testing for a measure of B-RAF inhibitor resistance comprises assaying for an activating AKT1 mutation.

24. The method of claim 23, wherein the activating AKT1 mutation is Q79K.


SEQUENCE LISTING (162 sequences)

SEQ ID NO: 1 - DNA, 21 bp, Homo sapiens
cgctgcctgmagtggaccact

SEQ ID NO: 2 - DNA, 20 bp, artificial sequence
  primer
cggcgacttctcgtcgtctc

SEQ ID NO: 3 - DNA, 24 bp, artificial sequence
  primer
ctggcagttactgtgatgtagttg

SEQ ID NO: 4 - DNA, 23 bp, artificial sequence
  primer
ggaccatctagatatcacatatg

SEQ ID NO: 5 - DNA, 24 bp, artificial sequence
  primer
gtagaaatggtgttgtatctgacc

SEQ ID NO: 6 - DNA, 25 bp, artificial sequence
  primer
cgatggaatattagggagccaaacc

SEQ ID NO: 7 - DNA, 24 bp, artificial sequence
  primer
gttgtcaagcttgaaatcagttgc

SEQ ID NO: 8 - DNA, 21 bp, artificial sequence
  primer
ctgagaatggaatttgatctc

SEQ ID NO: 9 - DNA, 21 bp, artificial sequence
  primer
cttgagcaaagcagctttggc

SEQ ID NO: 10 - DNA, 26 bp, artificial sequence
  primer
gtgtccacttgttcttatcattcagc

SEQ ID NO: 11 - DNA, 22 bp, artificial sequence
  primer
gtatgtgtctatgtctatcatc

SEQ ID NO: 12 - DNA, 23 bp, artificial sequence
  primer
ctcttcctgtatccctctcaggc

SEQ ID NO: 13 - DNA, 24 bp, artificial sequence
  primer
ctagtacaggaatatcattgttag

SEQ ID NO: 14 - DNA, 25 bp, artificial sequence
  primer
gtaggaggttagacttggcaattgc

SEQ ID NO: 15 - DNA, 22 bp, artificial sequence
  primer
cttgactggagtgaaaggtttg

SEQ ID NO: 16 - DNA, 25 bp, artificial sequence
  primr
gactctaagaggaaagatgaagtac

SEQ ID NO: 17 - DNA, 22 bp, artificial sequence
  primer
ctgtctctttctgagtatgtag

SEQ ID NO: 18 - DNA, 24 bp, artificial sequence
  primer
gtgggtttcccaccatctatgatg

SEQ ID NO: 19 - DNA, 26 bp, artificial sequence
  primer
gatttcaggtgctttcttgtaaagtg

SEQ ID NO: 20 - DNA, 20 bp, artificial sequence
  primer
ctgcatgacggagagggaca

SEQ ID NO: 21 - DNA, 27 bp, artificial sequence
  primer
cttcccaaatctattcctaatcccacc

SEQ ID NO: 22 - DNA, 25 bp, artificial sequence
  primer
cattcctgtatgacatggatgcctc

SEQ ID NO: 23 - DNA, 25 bp, artificial sequence
  primer
gatcaaagtaacaaaccctacagtc

SEQ ID NO: 24 - DNA, 23 bp, artificial sequence
  primer
ctaaacaaatgttggcctctagg

SEQ ID NO: 25 - DNA, 23 bp, artificial sequence
  primer
ctgtatagctgaaccagcattac

SEQ ID NO: 26 - DNA, 24 bp, artificial sequence
  primer
gcatgtcactgaagagcagaagtc

SEQ ID NO: 27 - DNA, 25 bp, artificial sequence
  primer
cagaagcttttctgatttgtgattc

SEQ ID NO: 28 - DNA, 25 bp, artificial sequence
  primer
gtttctctacacatttttctctgtg

SEQ ID NO: 29 - DNA, 22 bp, artificial sequence
  primer
gaattctgtgtcacatatggac

SEQ ID NO: 30 - DNA, 25 bp, artificial sequence
  primer
ggtaggagtcccgactgctgtgaac

SEQ ID NO: 31 - DNA, 26 bp, artificial sequence
  primer
ctatcagccataccatataacattgc

SEQ ID NO: 32 - DNA, 24 bp, artificial sequence
  primer
gttagcatccttatgttcctggac

SEQ ID NO: 33 - DNA, 23 bp, artificial sequence
  primer
caggctgtggtatcctgctctcc

SEQ ID NO: 34 - DNA, 25 bp, artificial sequence
  primer
gttgagaccttcaatgactttctag

SEQ ID NO: 35 - DNA, 24 bp, artificial sequence
  primer
ctatccttcacgcttacccaggag

SEQ ID NO: 36 - DNA, 25 bp, artificial sequence
  primer
gagtctgcacatagaatccaaactc

SEQ ID NO: 37 - DNA, 22 bp, artificial sequence
  primer
ccacacaagtgttctttggttc

SEQ ID NO: 38 - DNA, 23 bp, artificial sequence
  primer
agagggcagtaaggaggacttcc

SEQ ID NO: 39 - DNA, 21 bp, artificial sequence
  primer
acagacccacagctggtggtg

SEQ ID NO: 40 - DNA, 22 bp, artificial sequence
  primer
accgcccactgtcctgtggttc

SEQ ID NO: 41 - DNA, 21 bp, artificial sequence
  primer
tgatctcagccatcctggccc

SEQ ID NO: 42 - DNA, 22 bp, artificial sequence
  primer
atgtgtccttgaccggggagag

SEQ ID NO: 43 - DNA, 22 bp, artificial sequence
  primer
tacccagagctgcccatgaacg

SEQ ID NO: 44 - DNA, 22 bp, artificial sequence
  primer
acctccctgtccccaatggtgg

SEQ ID NO: 45 - DNA, 21 bp, artificial sequence
  primer
tctgccaccttcacgcgaacc

SEQ ID NO: 46 - DNA, 22 bp, artificial sequence
  primer
agtcatagggcagctgcatggg

SEQ ID NO: 47 - DNA, 23 bp, artificial sequence
  primer
tagttggaggactcgatgtctgc

SEQ ID NO: 48 - DNA, 23 bp, artificial sequence
  primer
agtctctcgagaagcagcaccag

SEQ ID NO: 49 - DNA, 23 bp, artificial sequence
  primer
agaaggggacagctgataagggc

SEQ ID NO: 50 - DNA, 25 bp, artificial sequence
  primer
taaagtactgtagatgtggctcgcc

SEQ ID NO: 51 - DNA, 34 bp, artificial sequence
  primer
ggcttgaatagttagatgcttatttaaccttggc

SEQ ID NO: 52 - DNA, 20 bp, artificial sequence
  primer
ccactgtacccagcctaatc

SEQ ID NO: 53 - DNA, 20 bp, artificial sequence
  primer
acaccagcccgtttatggct

SEQ ID NO: 54 - DNA, 27 bp, artificial sequence
  primer
acagaatatgggtaaagatgatccgac

SEQ ID NO: 55 - DNA, 28 bp, artificial sequence
  primer
gctctatcttccctagtgtggtaacctc

SEQ ID NO: 56 - DNA, 20 bp, artificial sequence
  primer
aagagacagaggctgcagtg

SEQ ID NO: 57 - DNA, 22 bp, artificial sequence
  primer
tgtgcagaagaggataggcaga

SEQ ID NO: 58 - DNA, 21 bp, artificial sequence
  primer
aaggtactggtggagtatttg

SEQ ID NO: 59 - DNA, 30 bp, artificial sequence
  primer
tgaagtaaaaggtgcactgtaataatccag

SEQ ID NO: 60 - DNA, 21 bp, artificial sequence
  primer
tgacaaaagttgtggacaggt

SEQ ID NO: 61 - DNA, 23 bp, artificial sequence
  primer
acaaaacacctatgcggatgaca

SEQ ID NO: 62 - DNA, 22 bp, artificial sequence
  primer
gtactcatgaaaatggtcagag

SEQ ID NO: 63 - DNA, 30 bp, artificial sequence
  primer
catttataaaacagggatattacctacctc

SEQ ID NO: 64 - DNA, 22 bp, artificial sequence
  primer
gcaatgccctctcaagagacaa

SEQ ID NO: 65 - DNA, 20 bp, artificial sequence
  primer
aacagtctgcatggagcagg

SEQ ID NO: 66 - DNA, 26 bp, artificial sequence
  primer
ggctgagcagggccctccttggcagg

SEQ ID NO: 67 - DNA, 29 bp, artificial sequence
  primer
ggtaccagggagaggctggctgtgtgaac

SEQ ID NO: 68 - DNA, 20 bp, artificial sequence
  primer
tacaggtgaaccccgtgagg

SEQ ID NO: 69 - DNA, 20 bp, artificial sequence
  primer
acctttgaggggctgctgta

SEQ ID NO: 70 - DNA, 25 bp, artificial sequence
  primer
gccctatcctggctgtgtcctgggc

SEQ ID NO: 71 - DNA, 24 bp, artificial sequence
  primer
cagcggcatccaggacatgcgcag

SEQ ID NO: 72 - DNA, 20 bp, artificial sequence
  primer
ggagagggtcagtgagtgct

SEQ ID NO: 73 - DNA, 19 bp, artificial sequence
  primer
cacaagggaggctgctgac

SEQ ID NO: 74 - DNA, 24 bp, artificial sequence
  primer
gctttctttccatgataggagtac

SEQ ID NO: 75 - DNA, 21 bp, artificial sequence
  primer
cctgtttctcctccctctacc

SEQ ID NO: 76 - DNA, 22 bp, artificial sequence
  primer
atcagtcttccttctaccctgg

SEQ ID NO: 77 - DNA, 20 bp, artificial sequence
  primer
acacccaccaggaatactgc

SEQ ID NO: 78 - DNA, 59 bp, artificial sequence
  ShRNA
tggaatctcattcgatgcatacttcaagagagtatgcatcgaatgagattccttttttc

SEQ ID NO: 79 - DNA, 63 bp, artificial sequence
  shRNA
tcgagaaaaaaggaatctcattcgatgcatactctcttgaagtatgcatcgaatgagattcca

SEQ ID NO: 80 - DNA, 55 bp, artificial sequence
  shRNA
tgagcgacggtggctacatgttcaagagacatgtagccaccgtcgctcttttttc

SEQ ID NO: 81 - DNA, 59 bp, artificial sequence
  shRNA
tcgagaaaaaagagcgacggtggctacatgtctcttgaacatgtagccaccgtcgctca

SEQ ID NO: 82 - DNA, 55 bp, artificial sequence
  shRNA
tgaagccacgttacgagatcttcaagagagatctcgtaacgtggcttcttttttc

SEQ ID NO: 83 - DNA, 59 bp, artificial sequence
  shRNA
tcgagaaaaaagaagccacgttacgagatctctcttgaagatctcgtaacgtggcttca

SEQ ID NO: 84 - DNA, 53 bp, artificial sequence
  shRNA
tggtgggcacactacaatttccacaccaaattgtagtgtgcccaccttttttc

SEQ ID NO: 85 - DNA, 57 bp, artificial sequence
  shRNA
tcgagaaaaaaggtgggcacactacaatttggtgtggaaattgtagtgtgcccacca

SEQ ID NO: 86 - DNA, 55 bp, artificial sequence
  shRNA
tgagcagattaagcgagtaattcaagagattactcgcttaatctgctcttttttc

SEQ ID NO: 87 - DNA, 59 bp, artificial sequence
  shRNA
tcgagaaaaaagagcagattaagcgagtaatctcttgaattactcgcttaatctgctca

SEQ ID NO: 88 - DNA, 55 bp, artificial sequence
  shRNA
tgaaatacgccagtaccgaattcaagagattcggtactggcgtatttcttttttc

SEQ ID NO: 89 - DNA, 59 bp, artificial sequence
  shRNA
tcgagaaaaaagaaatacgccagtaccgaatctcttgaattcggtactggcgtatttca

SEQ ID NO: 90 - DNA, 55 bp, artificial sequence
  shRNA
tgtggtgatgtaacaagatattcaagagatatcttgttacatcaccacttttttc

SEQ ID NO: 91 - DNA, 59 bp, artificial sequence
  shRNA
tcgagaaaaaagtggtgatgtaacaagatatctcttgaatatcttgttacatcaccaca

SEQ ID NO: 92 - DNA, 55 bp, artificial sequence
  shRNA
tgcactgacaatccagctaattcaagagattagctggattgtcagtgcttttttc

SEQ ID NO: 93 - DNA, 59 bp, artificial sequence
  shRNA
tcgagaaaaaagcactgacaatccagctaatctcttgaattagctggattgtcagtgca

SEQ ID NO: 94 - DNA, 20 bp, artificial sequence
  primer
acagtgccatgagagaccaa

SEQ ID NO: 95 - DNA, 20 bp, artificial sequence
  primer
atgttgaatgtgacagcacc

SEQ ID NO: 96 - DNA, 20 bp, artificial sequence
  primer
ttccatgccgagtaacagac

SEQ ID NO: 97 - DNA, 20 bp, artificial sequence
  primer
gacagctcttccacccagag

SEQ ID NO: 98 - DNA, 20 bp, artificial sequence
  primer
caatgaccccttcattgacc

SEQ ID NO: 99 - DNA, 20 bp, artificial sequence
  primer
tcgcttaatctgctccctgt

SEQ ID NO: 100 - DNA, 20 bp, artificial sequence
  primer
ctcacaccactgggtaacaa

SEQ ID NO: 101 - DNA, 20 bp, artificial sequence
  primer
cgttggtgatcataggggac

SEQ ID NO: 102 - DNA, 20 bp, artificial sequence
  primer
tgaagtcctgtgcactggtc

SEQ ID NO: 103 - DNA, 20 bp, artificial sequence
  primer
gacaagcttcccgttctcag

SEQ ID NO: 104 - DNA, 20 bp, artificial sequence
  primer
ttggattgtgtccgttgagc

SEQ ID NO: 105 - DNA, 20 bp, artificial sequence
  primer
aattcaccccaccagtgcag

SEQ ID NO: 106 - DNA, 20 bp, artificial sequence
  primer
accctgagtcccatcatcac

SEQ ID NO: 107 - DNA, 20 bp, artificial sequence
  primer
cttcccgttctcagccttga

SEQ ID NO: 108 - DNA, 21 bp, artificial sequence
  primer
ggctctcggttataagatggc

SEQ ID NO: 109 - DNA, 20 bp, artificial sequence
  primer
acaggaaacgcaccatatcc

SEQ ID NO: 110 - DNA, 21 bp, artificial sequence
  primer
tcaatcatccacagagacctc

SEQ ID NO: 111 - DNA, 22 bp, artificial sequence
  primer
ggatccagacaactgttcaaac

SEQ ID NO: 112 - DNA, 20 bp, artificial sequence
  primer
acaaacagaggacagtggac

SEQ ID NO: 113 - DNA, 23 bp, artificial sequence
  primer
ttagttagtgagccaggtaatga

SEQ ID NO: 114 - RNA, 21 nt, artificial sequence
  siRNA
ggacaguggacuugauuaguu

SEQ ID NO: 115 - RNA, 21 nt, artificial sequence
  siRNA
aggacaguggacuugauuauu

SEQ ID NO: 116 - RNA, 21 nt, artificial sequence
  siRNA
acugauauuuccuggcuuauu

SEQ ID NO: 117 - RNA, 21 nt, artificial sequence
  siRNA
cugucaaacaugugguuauuu

SEQ ID NO: 118 - DNA, 20 bp, artificial sequence
  primer
ttccatgccgagtaacagac

SEQ ID NO: 119 - DNA, 20 bp, artificial sequence
  primer
ccgcagacacctacaacatc

SEQ ID NO: 120 - DNA, 20 bp, artificial sequence
  primer
cccctggaagctgacttaca

SEQ ID NO: 121 - DNA, 20 bp, artificial sequence
  primer
atgttgaatgtgacagcacc

SEQ ID NO: 122 - DNA, 21 bp, artificial sequence
  primer
tgccattccggaggagaaaac

SEQ ID NO: 123 - DNA, 20 bp, artificial sequence
  primer
gacagctcttccacccagag

SEQ ID NO: 124 - DNA, 20 bp, artificial sequence
  primer
caatgaccccttcattgacc

SEQ ID NO: 125 - DNA, 20 bp, artificial sequence
  primer
cgttggtgatcataggggac

SEQ ID NO: 126 - DNA, 20 bp, artificial sequence
  primer
caatgtgaaaggccgaaggt

SEQ ID NO: 127 - DNA, 20 bp, artificial sequence
  primer
ctgggatcagtttacacgcc

SEQ ID NO: 128 - DNA, 20 bp, artificial sequence
  primer
ctcacaccactgggtaacaa

SEQ ID NO: 129 - DNA, 21 bp, artificial sequence
  primer
aggcttgtaactgctgaggtg

SEQ ID NO: 130 - DNA, 20 bp, artificial sequence
  primer
tgaagtcctgtgcactggtc

SEQ ID NO: 131 - DNA, 20 bp, artificial sequence
  primer
gacaagcttcccgttctcag

SEQ ID NO: 132 - DNA, 20 bp, artificial sequence
  primer
acctcagcagttacaagcct

SEQ ID NO: 133 - DNA, 21 bp, artificial sequence
  primer
gatattgcacgacagactgca

SEQ ID NO: 134 - DNA, 20 bp, artificial sequence
  primer
aattcaccccaccagtgcag

SEQ ID NO: 135 - DNA, 20 bp, artificial sequence
  primer
aattcaccccaccagtgcag

SEQ ID NO: 136 - DNA, 20 bp, artificial sequence
  primer
cactgggaaccaggagctaa

SEQ ID NO: 137 - DNA, 22 bp, artificial seqquence
  primer
agcatccttatgttcctggaca

SEQ ID NO: 138 - DNA, 20 bp, artificial sequence
  primer
cttcccgttctcagccttga

SEQ ID NO: 139 - DNA, 24 bp, artificial sequence
  shRNA
tggaatctcattcgatgcatactt

SEQ ID NO: 140 - DNA, 35 bp, artificial sequence
  shRNA
caagagagtatgcatcgaatgagattccttttttc

SEQ ID NO: 141 - DNA, 23 bp, artificial sequence
  shRNA
tgacagagagattcaagctattt

SEQ ID NO: 142 - DNA, 33 bp, artificial sequence
  shRNA
caagagaatagcttgaatctctctgtttttttc

SEQ ID NO: 143 - DNA, 23 bp, artificial sequence
  shRNA
tgcaaagaacatcatccatagtt

SEQ ID NO: 144 - DNA, 33 bp, artificial sequence
  shRNA
caagagactatggatgatgttctttgttttttc

SEQ ID NO: 145 - DNA, 23 bp, artificial sequence
  shRNA
tgacagagacctcaagagtaatt

SEQ ID NO: 146 - DNA, 33 bp, artificial sequence
  shRNA
caagagattactcttgaggtctctgtttttttc

SEQ ID NO: 147 - DNA, 23 bp, artificial sequence
  shRNA
tgcaacaacagggaccagatatt

SEQ ID NO: 148 - DNA, 33 bp, artificial sequence
  shRNA
caagagatatctggtccctgttgttgttttttc

SEQ ID NO: 149 - DNA, 24 bp, artificial sequence
  shRNA
tcgagaaaaaaggaatctcattcg

SEQ ID NO: 150 - DNA, 39 bp, artificial sequence
  shRNA
atgcatactctcttgaagtatgcatcgaatgagattcca

SEQ ID NO: 151 - DNA, 24 bp, artificial sequence
  shRNA
tcgagaaaaaaacagagagattca

SEQ ID NO: 152 - DNA, 36 bp, artificial sequence
  shRNA
agctattctcttgaaatagcttgaatctctctgtca

SEQ ID NO: 153 - DNA, 25 bp, artificial sequence
  shRNA
tcgagaaaaaacaaagaacatcatc

SEQ ID NO: 154 - DNA, 35 bp, artificial sequence
  shRNA
catagtctcttgaactatggatgatgttctttgca

SEQ ID NO: 155 - DNA, 25 bp, artificial sequence
  shRNA
tcgagaaaaaaacagagacctcaag

SEQ ID NO: 156 - DNA, 35 bp, artificial sequence
  shRNA
agtaatctcttgaattactcttgaggtctctgtca

SEQ ID NO: 157 - DNA, 25 bp, artificial sequence
  shRNA
tcgagaaaaaacaacaacagggacc

SEQ ID NO: 158 - DNA, 35 bp, artificial sequence
  shRNA
agatatctcttgaatatctggtccctgttgttgca

SEQ ID NO: 159 - DNA, 21 bp, Homo sapiens
acagctggamaagaagagtac

SEQ ID NO: 160 - DNA, 17 bp, Homo sapiens
ggacagtggtacctgca

SEQ ID NO: 161 - DNA, 16 bp, Homo sapiens
ggacagtgaaacactt

SEQ ID NO: 162 - DNA, 18 bp, Homo sapiens
ccggaggagaaaacactt